(19) United States
(12) Patent Application Publication
(10) Pub. No.: US 2024/0374757 A1
(43) Pub. Date: Nov. 14, 2024

(54) CASRX/CAS13D SYSTEMS TARGETING C9ORF72

(71) Applicant: UCL Business Ltd, London (GB)

(72) Inventors: Liam KEMPTHORNE, London (GB); Adrian ISAACS, London (GB)

(73) Assignee: UCL Business Ltd, London (GB)

(21) Appl. No.: 18/554,454

(22) PCT Filed: Apr. 19, 2022
(86) PCT No.: PCT/EP2022/060296

§ 371 Date: Oct. 6, 2023

(30) Foreign Application Priority Data
Apr. 16, 2021 (GB) 2105455.6

(51) Int. Cl. A61K 48/00; C12N 9/22; C12N 15/11; C12N 15/86

(52) U.S. Cl. CPC A61K 48/005; C12N 9/22; C12N 15/111; C12N 15/86; C12N 2310/20; C12N 2740/15043; C12N 2750/14143

(57) ABSTRACT

Provided herein is a composition comprising (i) a nucleic acid sequence encoding a CasRx/Cas13d polypeptide; and (ii) a guide RNA that binds specifically to a target sequence in C9orf72 RNA. Also provided are associated pharmaceutical compositions, guide RNAs, complexes, vectors and cells, and uses of the compositions to neurodegenerative disorders.

### FIELD OF THE INVENTION

The present invention relates to gene therapy treatments for C9orf72-mediated diseases, such as frontotemporal dementia (FTD) and amyotrophic lateral sclerosis (ALS).

### BACKGROUND OF THE INVENTION

Frontotemporal dementia (FTD) and amyotrophic lateral sclerosis (ALS) are two inexorable neurodegenerative disorders. FTD patients present with gradual behavioural and cognitive impairments associated with neuronal atrophy of the frontal and temporal lobes. ALS patients typically present with progressive muscular weakness, eventually leading to paralysis due to loss of upper and lower motor neurons (Ferrari et al. (2011). FTD and ALS: a tale of two diseases. Current Alzheimer Research, 8(3), 273-294. https://doi.org/10.2174/156720511795563700, and Ling et al. (2013). Converging mechanisms in ALS and FTD: Disrupted RNA and protein homeostasis. Neuron, 79(3), 416-438. https://doi.org/10.1016/j.neuron.2013.07.033). While ALS and FTD have seemingly distinct clinical presentations, 15% of ALS patients develop typical FTD symptoms such as behavioural and cognitive abnormalities. Similarly, 15% of FTD patients go on to develop motor function impairments indicative of ALS (Ringholz et al. (2005). Prevalence and patterns of cognitive impairment in sporadic ALS. Neurology, 65(4), 586 LP-590. https://doi.org/10.1212/01.wnl.0000172911.39167.b6, and Wheaton et al. (2007) Cognitive impairment in familial ALS. Neurology, 69(14), 1411 LP-1417. https://doi.org/10.1212/01.wnl.0000277422.11236.2c).

More recent understanding of the genetics and pathology of these two disorders illustrates that they exist on a common disease spectrum. An important discovery came with the identification of TDP-43 ubiquitinated inclusions found in both FTD and ALS, with mutations in TARDBP (the gene encoding TDP-43) causing primarily familial ALS, but also familial FTD (Borroni et al. (2009). Mutation within TARDBP leads to frontotemporal dementia without motor neuron disease. Human Mutation, 30(11). https://doi.org/10.1002/humu.21100, Hasegawa et al. (2008). Phosphorylated TDP-43 in frontotemporal lobar degeneration and amyotrophic lateral sclerosis. Annals of Neurology, 64(1), 60-70. https://doi.org/10.1002/ana.21425, Kabashi et al. (2008) TARDBP mutations in individuals with sporadic and familial amyotrophic lateral sclerosis. Nature Genetics, 40(5), 572-574. https://doi.org/10.1038/ng.132). While the majority of ALS and FTD cases are sporadic, roughly 10% of ALS and up to 50% of FTD of cases are familial, with mutations found in SOD1, FUS, and TARDBP shown to cause ALS; and mutations in MAPT, PGRN, VCP, and (HMP2B shown to cause FTD (Baker et al. (2006). Mutations in progranulin cause tau-negative frontotemporal dementia linked to chromosome 17. Nature, 442(7105), 916-919. https://doi.org/10.1038/nature05016, Hutton et al. (1998). Association of missense and 5′-splice-site mutations in tau with the inherited dementia FTDP-17. Nature, 393(June), 702-705. doi: 10.1038/31508. Kabashi et al., 2008; Parkinson et al. (2006). ALS phenotypes with mutations in CHMP2B (charged multivesicular body protein 2B). Neurology, 67(6), 1074-1077. https://doi.org/10.1212/01.wnl.0000231510.89311.8b, Rosen et al. (1993). Mutations in Cu/Zn superoxide dismutase gene are associated with familial amyotrophic lateral sclerosis. Nature, 362(6415), 59-62. https://doi.org/10.1038/362059a0). Although across all mutations there is clinical overlap between ALS and FTD in rare cases.

Until relatively recently, many familial cases had no known mutation. In 2011, a G₄C₂ hexanucleotide repeat expansion in a non-coding region of Chromosome 9 open reading frame 72 (C9orf72) was discovered to be the most common cause of familial FTD and ALS cases in Caucasian populations, accounting for 25% and 40% of familial FTD and ALS respectively (DeJesus-Hernandez et al. (2011). Expanded GGGGCC Hexanucleotide Repeat in Noncoding Region of C9ORF72 Causes Chromosome 9p-Linked FTD and ALS. Neuron, 72(2), 245-256. https://doi.org/10.1016/j.neuron.2011.09.011, Majounie et al. (2012). Frequency of the C9orf72 hexanucleotide repeat expansion in patients with amyotrophic lateral sclerosis and frontotemporal dementia: A cross-sectional study. The Lancet Neurology, 11(4), 323-330. https://doi.org/10.1016/S1474-4422(12)70043-1, Renton et al. (2011). A hexanucleotide repeat expansion in C9ORF72 is the cause of chromosome 9p21-linked ALS-FTD. Neuron, 72(2), 257-268. https://doi.org/10.1016/j.neuron.2011.09.010). In addition to FTD and ALS clinical indicators, C9orf72 FTD/ALS patients may suffer from neuropsychiatric symptoms and Parkinsonism (Cooper-Knock et al. (2014) The widening spectrum of C9ORF72-related disease; genotype/phenotype correlations and potential modifiers of clinical phenotype. Acta Neuropathologica, 127(3), 333-345. https://doi.org/10.1007/s00401-014-1251-9). C9orf72 patients have been diagnosed as Alzheimer, progressive supranuclear palsy, and Huntington disease patients further highlighting a clinical heterogeneity (Woollacott & Mead. (2014). The C9ORF72 expansion mutation: gene structure, phenotypic and diagnostic issues. Acta Neuropathologica, 127(3), 319-332. https://doi.org/10.1007/s00401-014-1253-7).

C9orf72 FTD/ALS patients may harbour thousands of G₄C₂ repeats compared to a median of 2 repeats in the general population. The hexanucleotide repeat expansion lies in intron 1 of the C9orf72 gene within the promoter region of variant 2 and is part of the pre-mRNA of C9orf72 variants 1 and 3 (FIG. 1; Balendra & Isaacs. (2018). C9orf72-mediated ALS and FTD: multiple pathways to disease. Nature Reviews Neurology, 14(9), 544-558. https://doi.org/10.1038/s41582-018-0047-2). These transcripts lead to the expression of two protein isoforms with variant 2 and the long isoform of C9orf72 being the highest expressed in the central nervous system (CNS) (FIG. 1; Rizzu et al. (2016). C9orf72 is differentially expressed in the central nervous system and myeloid cells and consistently reduced in C9orf72, MAPT and GRN mutation carriers. Acta Neuropathologica Communications, 4(1), 37. https://doi.org/10.1186/s40478-016-0306-7).

Both loss and gain of function mechanisms have been proposed as pathogenic processes in C9orf72 FTD/ALS, with recent evidence suggesting these mechanisms act synergistically in disease pathogenesis (Zhu et al. (2020). Reduced C9ORF72 function exacerbates gain of toxicity from ALS/FTD-causing repeat expansion in C9orf72. Nature Neuroscience, 23:615-624. https://doi.org/10.1038/s41593-020-0619-5). Indeed, the majority of evidence suggests that C9orf72-related FTD/ALS is caused by a toxic gain of function (Mizielinska et al. (2014). C9orf72 repeat expansions cause neurodegeneration in Drosophila through arginine-rich proteins. Science, 6201, 1192-1194. https://doi.org/10.1126/science.1256800, Saberi et al. (2017). Sense-encoded poly-GR dipeptide repeat proteins correlate to neurodegeneration and uniquely co-localize with TDP-43 in dendrites of repeat-expanded C9orf72 amyotrophic lateral sclerosis. Acta Neuropathologica, 1-16. https://doi.org/10.1007/s00401-017-1793-8, however C9orf72 patients have a reduced expression of C9orf72 (˜50%) suggesting a potential loss of function contribution to disease pathogenesis (Jackson et al. (2020). Elevated methylation levels, reduced expression levels, and frequent contractions in a clinical cohort of C9orf72 expansion carriers. Molecular Neurodegeneration, 15(1), 1-11. https://doi.org/10.1186/s13024-020-0359-8, Rizzu et al., 2016). C9orf72 is a suggested guanine exchange factor that has been implicated in the regulation of autophagy via the activation of Rab proteins (Iyer et al. (2018). C9orf72, a protein associated with amyotrophic lateral sclerosis (ALS) is a guanine nucleotide exchange factor. Peer J, 6: e5815.https://doi.org/10.7717/peerj.5815). C9orf72 FTD/ALS patients have reduced mRNA and protein levels of C9orf72 long and short isoforms due to the presence of the hexanucleotide expansion repeat (Rizzu et al., 2016). Loss of C9orf72 has been shown to impair autophagy, lysosomal biogenesis, and vesicular trafficking in cell models, with one report of C9orf72 haploinsufficiency leading to neurodegeneration in human-derived cell models (Shi et al. (2018). Haploinsufficiency leads to neurodegeneration in C9ORF72 ALS/FTD human induced motor neurons. Nature Medicine, 24(3), 313-325. https://doi.org/10.1038/nm.4490, Webster et al. (2016). The C9orf72 protein interacts with Rabla and the ULK 1 complex to regulate initiation of autophagy. The EMBO Journal, 35(15): 1656-76. doi: 10.15252/embj.201694401). Whilst C9orf72-knockout mice do not exhibit neurodegeneration or motor dysfunction, they do develop splenomegaly and exhibit peripheral and CNS immune cell deficits (Burberry et al. (2016). Loss-of-function mutations in the C9ORF72 mouse ortholog cause fatal autoimmune disease. Science Translational Medicine, 8(347). https://doi.org/10.1126/scitranslmed.aaf6038, Koppers et al. (2015). C9orf72 ablation in mice does not cause motor neuron degeneration or motor deficits. Annals of Neurology, 78(3), 426-438. https://doi.org/10.1002/ana.24453, O'Rourke et al. (2016). C9orf72 is required for proper macrophage and microglial function in mice. Science (New York, N.Y.), 351(6279), 1324-1329. https://doi.org/10.1126/science.aaf1064, Sareen et al. (2013). Targeting RNA Foci in iPSC-Derived Motor Neurons from ALS Patients with a C9ORF72 Repeat Expansion. Science Translational Medicine, 5(208): 208ra149. doi: 10.1126/scitranslmed.3007529, Sudria-Lopez et al. (2016). Full ablation of C9orf72 in mice causes immune system-related pathology and neoplastic events but no motor neuron defects. Acta Neuropathologica, 132(1), 145-147. https://doi.org/10.1007/s00401-016-1581-x); however, it is not clear whether a ˜50% reduction in C9orf72, as is seen in patients, will lead to these pathologies. Perhaps more crucially, loss or reduction of C9orf72 function has been shown to exacerbate the gain of function mechanisms of the hexanucleotide expansion repeat with increased DPR accumulation, glial activation, and hippocampal neuron loss in a mouse model (Zhu et al., 2020). Therefore, an important part of any therapy should be to minimise any further reduction in C9orf72 expression.

The C9orf72 hexanucleotide repeat expansion undergoes bidirectional transcription to produce both sense and antisense repeat-containing transcripts which form sense and antisense RNA foci (Mizielinska et al. (2013). C9orf72 frontotemporal lobar degeneration is characterised by frequent neuronal sense and antisense RNA foci. Acta Neuropathologica, 126(6), 845-857.https://doi.org/10.1007/s00401-013-1200-z). Additionally, these transcripts have been shown to undergo repeat associated non-ATG (RAN) translation in all three frames, producing 5 distinct dipeptide repeat protein (DPR) species (FIG. 2; Mori et al. (2013). Bidirectional transcripts of the expanded C9orf72 hexanucleotide repeat are translated into aggregating dipeptide repeat proteins. Acta Neuropathologica, 126(6): 881-893. doi: 10.1007/s00401-013-1189-3).

There is strong evidence to suggest DPRs are toxic and a key pathogenic feature of the C9orf72 hexanucleotide repeat expansion with arginine-rich DPRs, poly-GR and poly-PR, but not repeat-containing RNA, associated with neurodegeneration in Drosophila and cellular models (Kanekura et al. (2016). Poly-dipeptides encoded by the C9ORF72 repeats block global protein translation. Human Molecular Genetics, 25(9), 1803-1813. https://doi.org/10.1093/hmg/ddw052, Mizielinska et al., 2014; Tran et al. (2015). Differential Toxicity of Nuclear RNA Foci versus Dipeptide Repeat Proteins in a Drosophila Model of C9ORF72 FTD/ALS. Neuron, 87(6), 1207-1214. https://doi.org/10.1016/j.neuron.2015.09.015, Wen et al. (2014). Antisense proline-arginine RAN dipeptides linked to C9ORF72-ALS/FTD form toxic nuclear aggregates that initiate in vitro and in vivo neuronal death. Neuron, 84(6), 1213-1225. https://doi.org/10.1016/j.neuron.2014.12.010). Additionally, poly-GR has been shown to correlate to neurodegeneration and co-localise and TDP-43 inclusions in C9orf72 patients (Saberi et al. (2018). Sense-encoded poly-GR dipeptide repeat proteins correlate to neurodegeneration and uniquely co-localize with TDP-43 in dendrites of repeat-expanded C9orf72 amyotrophic lateral sclerosis. Acta Neuropathologica, 135(3), 459-474. https://doi.org/10.1007/s00401-017-1793-8). Poly-GA has also been shown to be toxic in primary neurons, with a poly-GA expressing mouse model shown to develop neurodegeneration (Zhang et al. (2016). C9ORF72 poly (GA) aggregates sequester and impair HR23 and nucleocytoplasmic transport proteins. Nature Neuroscience, 19(5), 668-677. https://doi.org/10.1038/nn.4272).

RNA foci formed of both the sense G₄C₂ and antisense C₄G₂ transcripts are also a key pathologic feature of C9orf72 hexanucleotide expansion repeat (Mizielinska et al., 2013). While it is clear that the RNA foci sequester RNA binding proteins, there is evidence for and against the toxicity of the RNA foci (Moens et al. (2018). Sense and antisense RNA are not toxic in Drosophila models of C9orf72-associated ALS/FTD. Acta Neuropathologica, 135(3): 445-457. https://doi.org/10.1007/s00401-017-1798-3, Swinnen et al. (2018). A zebrafish model for C9orf72 ALS reveals RNA toxicity as a pathogenic mechanism. Acta Neuropathologica, 135(3), 427-443. https://doi.org/10.1007/s00401-017-1796-5, Xu et al. (2013). Expanded GGGGCC repeat RNA associated with amyotrophic lateral sclerosis and frontotemporal dementia causes neurodegeneration. Proceedings of the National Academy of Sciences of the United States of America, 110(19), 7778-7783. https://doi.org/10.1073/pnas. 1219643110).

A number of genetic C9orf72 knockdown strategies have been tested including using an enzymatically dead Cas9, RNA interference, and antisense oligonucleotides (ASOs) (Batra et al. (2017). Elimination of Toxic Microsatellite Repeat Expansion RNA by RNA-Targeting Cas9. Cell, 170(5), 899-912.e10. https://doi.org/10.1016/j.cell.2017.07.010, Donnelly et al. (2013). Article RNA Toxicity from the ALS/FTD C9ORF72 Expansion Is Mitigated by Antisense Intervention. NEURON, 80(2), 415-428. https://doi.org/10.1016/j.neuron.2013.10.015,, Jiang et al. (2016). Gain of Toxicity from ALS/FTD-Linked Repeat Expansions in C9ORF72 Is Alleviated by Antisense Oligonucleotides Targeting GGGGCC-Containing RNAs. Neuron, 90(3), 535-550. https://doi.org/10.1016/j.neuron.2016.04.006. Martier et al. (2019). Targeting RNA-Mediated Toxicity in C9orf72 ALS and/or FTD by RNAi-Based Gene Therapy. Molecular Therapy—Nucleic Acids, 16(June), 26-37. https://doi.org/10.1016/j.omtn.2019.02.001, Sareen et al., 2013). ASOs targeting the sense C9orf72 transcript are currently the most developed with a clinical trial underway to determine efficacy and safety in C9orf72 ALS patients (clinicaltrials.gov: NCT03626012). However, current ASO therapies do not readily cross an intact blood-brain barrier, therefore repeated application via intrathecal injection is required. As ASOs require multiple administrations per year, a lifetime course of treatment becomes extremely expensive. For example, the cost of FDA-approved ASOs for spinal muscular atrophy costs $750,000 in the first year and approximately $375,000 annually for life (Krishnan & Mishra. (2020). Antisense Oligonucleotides: A Unique Treatment Approach. Indian Pediatrics, 57(2), 165-171. https://doi.org/10.1007/s13312-020-1736-7, Wurster & Ludolph. (2018). Nusinersen for spinal muscular atrophy. Therapeutic Advances in Neurological Disorders, 11, 175628561875445. https://doi.org/10.1177/1756285618754459). Additionally, ASOs currently in clinical trial only target the sense repeat containing transcripts. This approach leaves the antisense repeat-containing transcripts unaltered, resulting in the expression of toxic poly-PR (Mizielinska et al. (2014). C9orf72 repeat expansions cause neurodegeneration in Drosophila through arginine-rich proteins. Science, 345(6201), 1192-1195. doi: 10.1126/science.1256800). There are currently no gene therapy strategies for C9orf72 that can target both sense and antisense pathology, despite antisense pathology being present in patients' brains (Mizielinska et al., 2013). Whilst it is known where the transcription start sites for the sense transcripts are, and it is even known that the sense transcripts form G-quadruplex and hairpin RNA secondary structures, much less is known about the antisense transcript making it difficult to therapeutically target (Fratta et al. (2012). C9orf72 hexanucleotide repeat associated with amyotrophic lateral sclerosis and frontotemporal dementia forms RNA G-quadruplexes. Scientific Reports, 2, 1-6. https://doi.org/10.1038/srep01016). There is therefore a need for safer, cheaper and more effective treatments for C9orf72-related diseases, and in particular, treatments that target both the sense and antisense pathologies.

Clustered regularly interspaced short palindromic repeat (CRISPR) RNAs and CRISPR-associated (Cas) proteins are part of the adaptive immunity of bacteria. The harnessing of DNA engineering CRISPR-Cas9 systems revolutionised genetic manipulation research and allowed researchers to target and alter genes and correct disease-causing mutations (Doudna & Charpentier. (2014). Genome editing. The new frontier of genome engineering with CRISPR-Cas9. Science (New York, N.Y.), 346(6213), 1258096. https://doi.org/10.1126/science.1258096, Hsu et al. (2014). Development and applications of CRISPR-Cas9 for genome engineering. Cell, 157(6), 1262-1278. https://doi.org/10.1016/j.cell.2014.05.010, Ran et al. (2013). Genome engineering using the CRISPR-Cas9 system. Nature Protocols, 8(11), 2281-2308. https://doi.org/10.1038/nprot.2013.143). However, in practice, therapies that alter the genome have been hard to optimise to a safe and efficacious level, with any off-target effects being permanent (Peng et al. (2016). Potential pitfalls of CRISPR/Cas9-mediated genome editing. FEBS Journal, 283(7), 1218-1231. https://doi.org/10.1111/febs.13586). There is therefore a need for improved treatments for targeting C9orf72-mediated pathology for treatment in diseases such as C9orf72 FTD/ALS.

### SUMMARY OF THE INVENTION

Accordingly, in one aspect the present invention provides a composition comprising:
(i) a nucleic acid sequence encoding a CasRx/Cas13d polypeptide; and
(ii) one or more guide RNAs that bind specifically to a target sequence in C9orf72 RNA.

In one embodiment, the one or more guide RNAs bind to, associates with or forms a complex with the CasRx/Cas13d polypeptide and directs specific cleavage and/or degradation of C9orf72 RNA.

In one embodiment, the target sequence is present in a sense C9orf72 RNA, e.g. a sense C9orf72 transcript, pre-mRNA or mRNA.

In one embodiment, the one or more guide RNAs direct CasRx/Cas13d-mediated cleavage and/or degradation of a sense C9orf72 RNA, e.g. a sense C9orf72 transcript, pre-mRNA or mRNA.

In one embodiment, the target sequence is present in a sense C9orf72 RNA transcript at a position corresponding to or within base pairs 150-400 of the C9orf72 gene (as shown in SEQ ID NO: 56). In one embodiment, the target sequence is present in a sense C9orf72 RNA transcript at a position corresponding to or within base pairs 150-350, 200-350, or 200-320 of the C9orf72 gene (as shown in SEQ ID NO: 56).

In one embodiment, the target sequence is present in an antisense C9orf72 RNA transcript. In one embodiment, the guide RNA directs Cas13d/CasRx to cleave and/or degrade an antisense C9orf72 RNA transcript.

In one embodiment, the target sequence is present in an antisense C9orf72 RNA transcript and is complementary to a sequence within base pairs 350-700 of the C9orf72 gene (as shown in SEQ ID NO: 56). In one embodiment, the target sequence is present in an antisense C9orf72 RNA transcript and is complementary to a sequence within base pairs 350-650, 400-700, 350-600, 400-650, 400-600, or 410-575 of the C9orf72 gene (as shown in SEQ ID NO: 56).

In one embodiment, the composition comprises a first guide RNA that binds specifically to, hybridizes to or is complementary to a target sequence in a sense C9orf72 RNA transcript, and a second guide RNA that binds specifically to, hybridizes to or is complementary to a target sequence in an antisense C9orf72 RNA transcript; and/or wherein the guide RNAs direct Cas13d/CasRx to cleave and/or degrade the sense and antisense C9orf72 transcripts.

In one embodiment, the target sequence is 5′ to a hexanucleotide repeat sequence in a sense C9orf72 transcript.

In one embodiment, the target sequence is 5′ to a hexanucleotide repeat sequence in intron 1 of C9orf72 pre-mRNA.

In one embodiment, the hexanucleotide repeat comprises the sequence (G₄C₂)ⁿ.

In one embodiment, the one or more guide RNAs preferentially bind to and/or directs specific cleavage and/or degradation of C9orf72 RNA variants 1 and/or 3.

In one embodiment, the one or more guide RNAs do not bind to and/or do not cleave and/or do not degrade C9orf72 transcript variant 2.

In one embodiment, the one or more guide RNAs comprise any one of SEQ ID NOs: 1-30.

In one embodiment, the one or more guide RNAs comprise any one of SEQ ID NOs: 1-3, or 22-30.

In one embodiment, the target sequence is 5′ to a hexanucleotide repeat sequence in an antisense C9orf72 RNA transcript.

In one embodiment, the hexanucleotide repeat comprises the sequence (C₄G₂)ₙ or (G₂C₄)ₙ.

In one embodiment, the one or more guide RNAs comprise any one of SEQ ID NOs: 31-45.

In one embodiment, the one or more guide RNAs comprise any one of SEQ ID NOs: 31-33, or 37-45.

In one embodiment, the one or more guide RNAs comprise one or more of SEQ ID NOs: 1-3, 22-33, or 37-45, or any combination thereof.

In a further aspect, the present invention provides a guide RNA that binds specifically to a target sequence in C9orf72 RNA, wherein the guide RNA is capable of binding to a CasRx/Cas13d polypeptide and directing specific cleavage and/or degradation of C9orf72 RNA.

In one embodiment, the guide RNA comprises a spacer sequence complementary to, or capable of specifically hybridizing to, the target sequence.

In one embodiment, the spacer sequence is selected from any one of SEQ ID NO:s 1, 4, 7, 10, 13, 16, 19, 22, 25 or 28. In a preferred embodiment, the spacer sequence is selected from any one of SEQ ID NO:s 1, 22, 25 or 28.

In one embodiment the spacer sequence is selected from any one of SEQ ID NO:s 31, 34, 37, 40 or 43. In a preferred embodiment, the spacer sequence is selected from any one of SEQ ID NOs: 31, 37, 40 or 43.

In one embodiment, the spacer sequence is selected from one or more of SEQ ID NOs: 1, 4, 7, 10, 13, 16, 19, 22, 25, 28, 31, 34, 37, 40 or 43, or any combination thereof. In a preferred embodiment, the spacer sequence is selected from one or more of SEQ ID NOs: 1, 22, 25, 28, 31, 37, 40 or 43, or any combination thereof.

In one embodiment, the guide RNA comprises a direct repeat sequence capable of binding to the CasRx/Cas13d polypeptide, preferably wherein the direct repeat sequence comprises SEQ ID NO: 46 or SEQ ID NO:47.

In a further aspect, the present invention provides a complex comprising:
(i) a CasRx/Cas13d polypeptide; and
(ii) one or more guide RNAs as defined above bound to the CasRx/Cas13d polypeptide.

In a further aspect, the present invention provides a vector comprising the composition, one or more guide RNAs or complex as defined above.

In one embodiment, the vector is an adeno-associated virus (AAV) or a lentivirus.

In a further aspect, the present invention provides a cell comprising the composition, one or more guide RNAs, complex or vector as defined above.

In a further aspect, the present invention provides a pharmaceutical composition comprising the composition, one or more guide RNAs, complex, vector or cell as defined above, and one or more pharmaceutically acceptable excipients, carriers or diluents.

In a further aspect, the present invention provides a composition, guide RNAs, complex, vector or cell as defined above, for use in preventing or treating a C9orf72-mediated disease, disorder or condition, preferably wherein the disease, disorder or condition is a neurodegenerative disorder.

In a further aspect, the present invention provides a composition, guide RNAs, complex, vector or cell as defined above, for use in preventing or treating a neurodegenerative disorder.

In one embodiment, the neurodegenerative disorder is frontotemporal dementia (FTD) or amyotrophic lateral sclerosis (ALS).

In a further aspect, the present invention provides a method of cleaving and/or degrading C9orf72 RNA in a preparation or cell, comprising contacting the preparation or cell with a composition, guide RNA, complex, vector or cell as defined above.

In one embodiment, the method selectively degrades C9orf72 pre-mRNA that comprises a hexanucleotide repeat expansion.

In a further aspect, the present invention provides a method of preventing or treating a C9orf72-mediated disease, disorder or condition in a subject in need thereof, wherein the method comprises administering to the subject a therapeutically effective amount of a composition, guide RNA, complex, vector or cell as defined above.

In a further aspect, the present invention provides a method of preventing or treating a neurodegenerative disorder in a subject in need thereof, wherein the method comprises administering to the subject a therapeutically effective amount of a composition, guide RNA, complex, vector or cell as defined above.

In one embodiment, the neurodegenerative disorder is frontotemporal dementia (FTD) or amyotrophic lateral sclerosis (ALS).

### BRIEF DESCRIPTION OF THE DRAWINGS

The accompanying drawings are not intended to be drawn to scale. The Figures are illustrative only and are not required for enablement of the disclosure. For purposes of clarity, not every component may be labelled in every drawing.

FIG. 1. C9orf72 gene, transcripts, and protein isoforms. The hexanucleotide expansion is located in intron 1 of the C9orf72 gene. C9orf72 is transcribed into three variants with the hexanucleotide repeats being positioned in intron 1 of variants 1 and 3, and the promoter region of variant 2. C9orf72 transcripts are therefore translated into two protein isoforms. Image sourced from Balendra & Isaacs, 2018.

FIG. 2: Dipeptide repeat proteins produced from sense and antisense C9orf72 transcripts. The C9orf72 hexanucleotide repeat expansion undergoes bidirectional transcription and repeat associated non-ATG translation producing 5 different dipeptide repeat proteins. Figure obtained from Balendra & Isaacs, 2018.

FIG. 3: Type VI Cas13 phylogenetic tree. Type VI CRIPSR-Cas13 orthologs discovered to date and their phylogenetic tree and commonly associated domains. Despite similar functions, type VI orthologs only share 11-16% homogeneity. Image sourced from Connell ((2019) Molecular Mechanisms of RNA Targeting by Type VI CRISPR—Cas Systems', Journal of Molecular Biology. Elsevier Ltd, 431(1), pp. 66-87. doi: 10.1016/j.jmb.2018.06.029).

FIG. 4: A schematic of the CasRx gRNA architecture. The pre-gRNA sequence is shown in red. This pre-gRNA sequence is the same for all of the guides. The target RNA sequence (i.e. proximal to the repeats) is indicated, and this is where the spacer guide sequence (bold) binds. Figure obtained from Connell (2019).

FIG. 5: Schematic of the RAN translated sense strand Nanoluciferase reporter plasmid (S92RNL). (A) Diagrammatic representation and (B) plasmid map of the S92RNL NanoLuc reporter assay. The sense Nanoluciferase reporter plasmid contains 92 pure G₄C₂ repeats with 120 nucleotides of the endogenous sequence upstream and a C-terminal Nanoluciferase in frame with a GR dipeptide repeat protein.

FIG. 6: Plasmid map of RAN translated antisense strand Nanoluciferase reporter plasmid (AS55RNL). (A) Diagrammatic representation and (B) plasmid map of the AS55RNL NanoLuc reporter assay. The Nanoluciferase antisense reporter plasmid contains ˜55 pure C₄G₂ repeats with 680 nucleotides of the endogenous 5′ sequence upstream and a C-terminal Nanoluciferase in frame with PR dipeptide repeat protein.

FIG. 7: Design of single U6-gRNA-Ef-1a-CasRx plasmids and lentiviruses. Diagrammatic representation of outcome plasmids of our cloning strategy to produce single lentiviral plasmids expressing both gRNA and CasRx.

FIG. 8: CasRx AAV therapy. Diagrammatic representation of CasRx and gRNA combined AAV therapy, together with gateway cloning sites allowing testing of different guide arrays.

FIG. 9: Cas13b can reduce poly-GR levels in a transient model in Hela cells. (A) Diagrammatic representation of the S92RNL NanoLuc reporter assay experiment. (B) 0 repeat and 92 repeat NLuc reporter assay comparing ten Cas13b gRNAs. Each NLuc reading was normalised to FLuc for each well and further normalised to the non-targeting control gRNA in the S92RNL assay. Data given as mean ±S.D. N=3 biological repeats. **** p<0.0001.

FIG. 10: CasRx is more efficient than Cas13b at preventing poly-GR formation and can reduce NLuc signal to background levels. (A) Diagrammatic representation of the different Cas13b and CasRx expression plasmids, with Cas13b previously shown to be more efficacious in the cytoplasm and therefore contains no nuclear localisation sequences (NLS). (B) Immunocytochemistry (ICC) for HA (Cas13b, CasRx or dCasRx) or imaging of GFP (CasRx or dCasRx only) showing transfection efficiency of Cas13b, CasRx, and dCasRx in HEK293T cells. Scale bars=20 μm. (C-E) Comparison of NLuc signal knockdown between Cas13b, CasRx and dCasRx in HEK293T cells. Each NLuc reading was normalised to FLuc for each well and further normalised to the non-targeting control gRNA. Data given as mean ±S.D. N=3 biological repeats with 3-4 technical replicated per biological replicate (all replicates given on graph). **** p<0.0001.

FIG. 11: CasRx can prevent sense RNA foci formation to background levels in a transient model indicated by RNA FISH and ICC. (A) RNA-FISH for the sense G₄C₂ transcript and ICC for the HA tag of CasRx with different CasRx guides. Scale bar=20 μm. (B) Quantification of RNA foci load calculated as total area of foci×foci signal intensity per CasRx positive cell. No repeat control indicates background signal of the LNA probe used for RNA FISH. N=2 biological replicates and 2 technical replicates per experiment. Technical replicates are displayed on the graph.

FIG. 12: CasRx can prevent antisense RNA foci formation and poly-PR accumulation in a transient model. (A) Antisense NLuc assay with NLuc as a reporter for poly-PR and testing of antisense transcript targeting gRNAs. Each NLuc reading was normalised to FLuc for each well and further normalised to the non-targeting control gRNA. Data given as mean ±S.D. N=3 biological repeats with 3-4 technical replicated per biological replicate (all replicates given on graph). **** p<0.0001. (B) RNA-FISH for the antisense G₄C₂ transcript and ICC for the HA tag of CasRx with different CasRx guides. Scale bar=20 μm. (C) Quantification of RNA foci load calculated as total area of foci×foci signal intensity per CasRx positive cell. No repeat control indicates background signal of the LNA probe used for RNA FISH. N=2 biological replicates and 3-5 technical replicates per experiment. Technical replicates are displayed on the graph.

FIG. 13: CasRx can mature our pre-gRNAs to gRNA and 30nt or 22nt guides are efficacious at reducing poly-GR or poly-PR. (A) Schematic illustrating the ability of CasRx to mature a pre-gRNA array adapted from Konermann et al. (2018. Transcriptome Engineering with RNA-Targeting Type VI-D CRISPR Effectors. Cell, 173(3), 665-668.e14. https://doi.org/10.1016/j.cell.2018.02.033). (B) Sense targeting guides cloned into a pre-gRNA expressing plasmid and tested in the S92RNL assay. (C-D) Testing of 30nt and 22nt gRNA variants of previously tested gRNAs in both the (C) sense and (D) antisense NLuc reporter assays. All NLuc data normalised to FLuc and non-targeting guide. Data given as mean ±S.D. N=3 biological repeats with 3-4 technical replicated per biological replicate (all replicates given on graph). **** p<0.0001.

FIG. 14: Design and testing of single U6-gRNA-Ef-1a-CasRx plasmids and lentiviruses in HEK293T and NPC cells. (A) Imaging of CasRx GFP in live HEK293T or NPCs cells transiently transfected with single plasmids expressing non-targeting guides (guide NT) or guide 8 and CasRx in the ‘forward’ orientation. (B) S92RNL assay testing ‘forward’ orientation single plasmids containing non-targeting guide or guide 8 and CasRx in HEK293T cells. N=1 biological repeat. (C-D) S92RNL and AS55RNL assays testing ‘forward’ orientation single plasmids for sense and antisense targeting guides respectively in HEK293T cells. N=2 biological repeats. All NLuc data normalised to FLuc and non-targeting guide. Data given as mean ±S.D. with 2-4 technical replicated per biological replicate (all replicates given on graph). (E) Lentiviral transduction of NPC cells with single lentivirus expressing guide 11 and CasRx. Scale bars=50 μm.

FIG. 15: CasRx targeting of C9orf72 transcripts reduces pathologic hallmarks of C9orf72 FTD/ALS in patient iPSC-derived neuronal progenitor cells. (A) Image panel illustrating transduction efficiency of different CasRx and gRNA expressing lentiviruses. (B) MSD for poly-GP in iPSC-derived NPCs treated with lentiviruses expressing CasRx and gRNAs. All data given as mean ±S.D. N=2 technical replicates. Scale bar=20 μm.

FIG. 16: C9orf72 BAC mice, which express detectable levels of poly-GA and poly-GP at 3 months of age. MSD of frozen mouse brains at 3 months of age illustrate expression of (A) poly-GA and (B) poly-GP. N=3-6 mice per group. ** p<0.01, *** p<0.001.

FIG. 17: Summary of differentiation of iPSCs into i3 cortical neurons (Fernandopulle et al. 2018).

FIG. 18: CRISPR-CasRx reduces sense DPR pathology and sense and antisense repeat containing transcripts in 3 patient lines of i3 neurons after 5 days. MSD of (A) poly-GA and (B) poly-GP in i3 neurons transduced with CRISPR-CasRx lentiviruses expressing targeting guide 8, guide 10, or non-targeting (NT) guide. qPCR analysis of FACs-sorted i3 neurons for (C) exon 1b containing transcripts, (D) sense repeat containing transcripts, or (E) antisense repeat containing transcripts. qPCR data analysed via 2\hat{ }-ΔΔCt method with GAPDH control. **** p<0.0001. N=3 independent inductions per line. N=3 patient lines.

FIG. 19: CRISPR-CasRx AAV can reduce C9orf72 149R repeat-containing RNA in vivo in a mouse model. Repeat containing transcript qPCR of frozen hippocampus of mice 3 weeks post injection at P0 with both CRISPR-CasRx AAV (either targeting guide 10 and 17 AAV or non-targeting control AAV) and C9orf72 149 repeat AAV. qPCR data analysed via 2\hat{ }-ΔΔCt method with GAPDH control. *** p<0.001. N=8 for mice injected with CRISPR-CasRx and non-targeting guides. N=14 for mice injected with CRISPR-CasRx, guide 10 and guide 17.

### DETAILED DESCRIPTION OF THE INVENTION

Unless otherwise defined below, all technical terms used herein have the same meaning as commonly understood by one of the ordinary skill in the art in the field to which this disclosure belongs.

Any reference to ‘or’ herein is intended to encompass ‘and/or’ unless otherwise stated.

As used herein, the singular forms ‘a’, ‘an’, and ‘the’ include both singular and plural referents unless the context clearly dictates otherwise.

The terms ‘comprising’, ‘comprises’ and ‘comprised of’ as used herein are synonymous with ‘including’, ‘includes’ or ‘containing’, ‘contains’, and are inclusive or open-ended and do not exclude additional, non-recited members, elements or method steps. The term also encompasses ‘consisting of’ and ‘consisting essentially of’.

Whereas the term ‘one or more’, such as one or more members of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

As used herein, the terms ‘ribonucleic acid molecule’, ‘RNA’ or ‘transcript’ refers to polymers of ribonucleotides (for example, at least 2, 3, 4, 5, 10, 15, 20, 25, 30, 50 or more ribonucleotides). As used herein, ‘RNA’ can refer to single stranded (ssRNA) or double-stranded RNA (dsRNA). This includes messenger RNA (mRNA), transfer RNA (RNA), ribosomal RNA (rRNA), non-coding RNA (ncRNAs), protein coding RNA (pcRNA), or antisense RNA. The term ‘RNA’ is also used herein to refer to precursors of RNA, such as pre-mRNA. RNA can be post transcriptionally modified and can be endogenous or chemically synthesized. As used herein, ‘mRNA’ refers to a single stranded RNA that is transcribed from a DNA sequence. ‘mRNA’ specifies the amino acid sequence of one or more polypeptide chains.

The term ‘nucleoside’ refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and 2,2N,N-dimethylguanosine (also referred to as rare nucleosides). The term ‘nucleotide’ refers to a nucleoside having one or more phosphate groups joined in ester linkages to the sugar moiety. Exemplary nucleotides include nucleoside monophosphates, diphosphates and triphosphates. The terms ‘polynucleotide’ and ‘nucleic acid molecule’ are used interchangeably herein and refer to a polymer of nucleotides joined together by a phosphodiester or phosphorothioate linkage between 5′ and 3′ carbon atoms, including DNA and RNA.

In alternative embodiments, the present nucleotide sequences may be modified to replace the intended RNA or DNA nucleotide with ‘nucleotide analogues’, ‘modified nucleotides’ or ‘altered nucleotides’ which are non-standard, non-naturally occurring ribonucleotides or deoxyribonucloetides. Exemplary nucleotide analogs are modified at any position so as to alter certain chemical properties of the nucleotide yet retain the ability of the nucleotide analog to perform its intended function. In addition, the phosphate group of the nucleotide may be modified by making substitutions which still allow the nucleotide to perform its intended function. These have been described extensively in the art and are very well known to a skilled person.

As used herein, the term ‘base pair’ refers to the interaction between pairs of nucleotides (or nucleotide analogs) on opposing strands of nucleotide sequences (e.g., a duplex formed by a strand of a guide RNA and a target RNA sequence), due primarily to H-bonding, van der Waals interactions, and the like between said nucleotides (or nucleotide analogs).

As used herein, ‘C9orf72’ refers to the Chromosome 9 open reading frame 72 (C9orf72) gene located on the short arm of chromosome 9 (9p21) in humans (Xu et al. (2021). Correlation between C9ORF72 mutation and neurodegenerative diseases: a comprehensive review of the literature. International Journal of Medical Sciences, 18(2): 378-386. doi: 10.7150/ijms.53550). The C9ORF72 gene encodes a protein which is highly conserved across species (DeJesus-Hernandez et al. (2011). Expanded GGGGCC Hexanucleotide Repeat in Noncoding Region of C9ORF72 Causes Chromosome 9p-Linked FTD and ALS. Neuron, 72(2), 245-256. https://doi.org/10.1016/j.neuron.2011.09.01). A nucleotide sequence of the coding (sense) strand of the C9orf72 gene is shown in SEQ ID NO: 56.

As used herein, the term ‘sense’ refers to a transcript, pre-mRNA or mRNA that encodes the C9orf72 protein in a 5′ to 3′ direction. Thus the sense transcript may contain an RNA sequence corresponding to a DNA sequence in the sense strand the C9orf72 gene, i.e. the normal coding sequence that is translated to a protein. The term “antisense” refers to a transcript, pre-mRNA or mRNA that may be complementary to the sense transcript, i.e. the antisense transcript is derived from transcription of the C9orf72 gene in a direction opposite to that of the sense transcript. Thus the antisense transcript may contain an RNA sequence corresponding to a DNA sequence in the antisense strand of the C9orf72 gene. The antisense does not encode the C9orf72 protein in a 5′ to 3′ direction, and is thus not translated to the C9orf72 protein.

The terms ‘hexanucleotide repeat’, ‘repeats’, or ‘hexanucleotide expansions’ as used herein refers to a sequence of six nucleotides (hence ‘hexanucleotide’) of GGGGCC (G₄C₂; SEQ ID NO: 62) in the sense DNA strand, or CCCCGG (C₄G₂; SEQ ID NO: 63) in the antisense DNA strand of the C9orf72 gene. The antisense hexanucleotide repeat may alternatively be represented as GGCCCC (G₂C₄; SEQ ID NO: 66), and thus C₄G₂ and G₂C₄ (SEQ ID NOs: 63 and 66) may be used herein interchangeably. The hexanucleotide sequence can occur only once or can be repeated multiple times (hence the term ‘repeats’ or ‘expansions’) (Xu et al., 2021). In embodiments, the hexanucleotide repeats are consecutive. In embodiments, the hexanucleotide repeats are interrupted by one or more nucleotides. The C9orf72 hexanucleotide expansions may be indicated as (G₄C₂)ₙ for sense or (C₄G₂)ₙ or (G₂C₄)ₙ for antisense expansions, respectively.

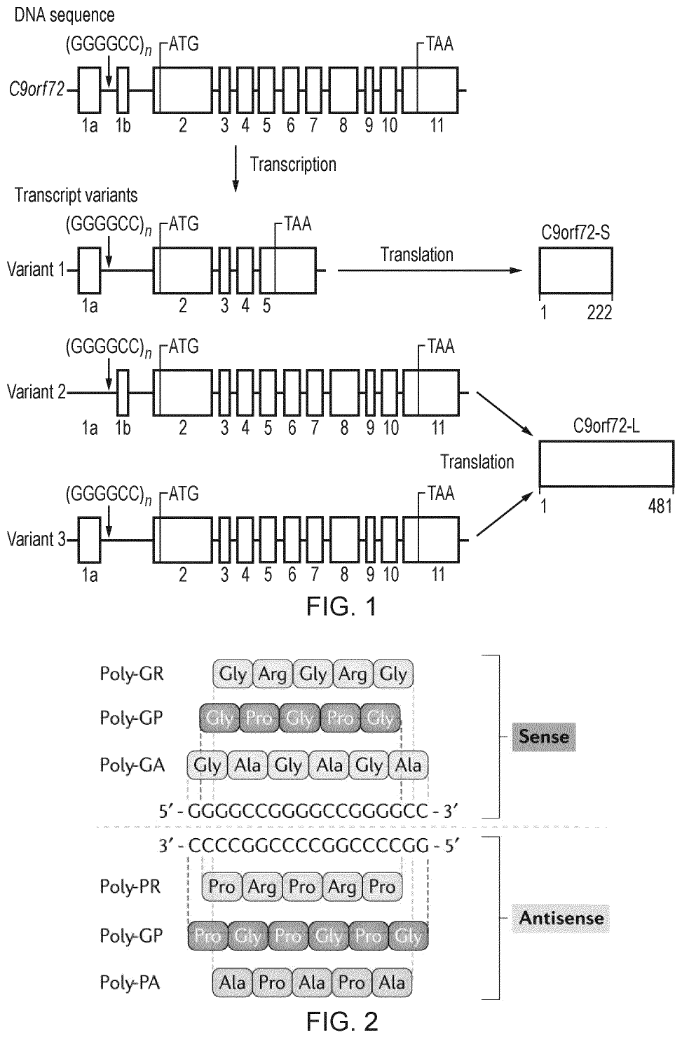

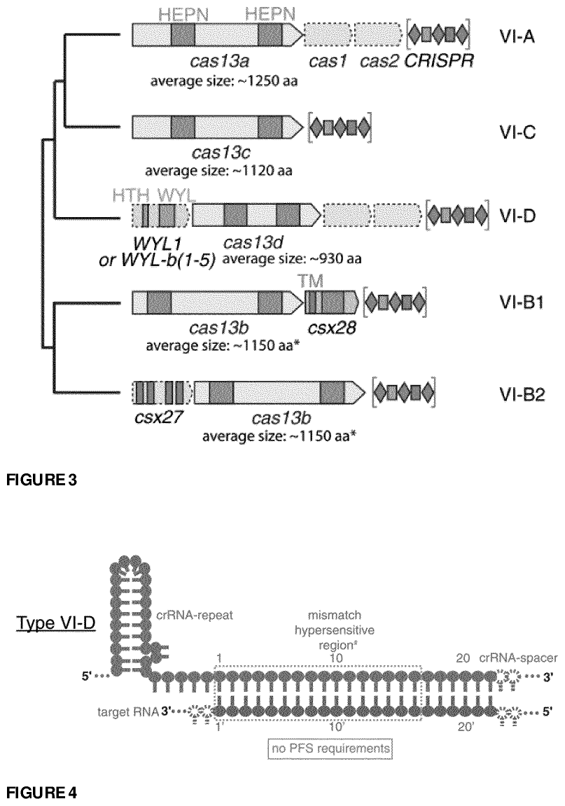

The C9orf72 hexanucleotide expansion is located in a non-coding region of the C9orf72 gene. Due to different transcription start sites, three different transcript variants are produced. The hexanucleotide expansion can be found either in the promoter region of the C9orf72 gene for variant 2, or in intron 1 of the C9orf72 gene for variants 1 and 3 (Balendra & Isaacs, 2018). As the hexanucleotide expansions are located in intron 1 for variants 1 and 3, the expansions for variants 1 and 2 are then also included in the respective pre-mRNAs. However, as the expansions are located in the promoter region for variant 2, the expansions are not incorporated into the pre-mRNA for variant 2 (FIG. 1; Balendra & Isaacs, 2018). The C9orf72 hexanucleotide repeat expansion undergoes bidirectional transcription, so transcripts can contain either sense (GGGGCC) or antisense (CCCCGG) expansions (Mizielinska et al., 2013). At the protein level, the variants then produce two different protein isoforms; transcript variant 1 produces the shorter sequence C9ORF72 protein subtype 1, consisting of 222 amino acids, while transcript variants 2 and 3 produce the longer C9ORF72 protein subtype 2,consisting of 481 amino acids (FIG. 2; Mori et al., 2013). In addition, despite being within a non-coding region of C9orf72, the RNA variants can be translated in every reading frame to form five different dipeptide repeat proteins (DPRs) containing the expansions via a non-canonical mechanism known as repeat-associated non-ATG (RAN) translation. The five resulting DPRs are poly-Gly-Ala (poly-GA), poly-Gly-Pro (poly-GP), and poly-Gly-Arg (Poly-GR) which are translated from the different open reading fragments of the sense transcript, whereas poly-GP, poly-Pro-Ala (poly-PA) and poly-Pro-Arg (poly-PR) are translated from the antisense transcript. As used herein ‘DPRs’ refers to the dipeptide repeat proteins of C9orf72 hexanucleotide expansions. As used herein, ‘DPRs’ refers to either poly-GA, -GR,-GP,-PA or -PR C9orf72 proteins.

As used herein, the terms ‘subject’, ‘patient’ or ‘individual’ are used interchangeably and refer to vertebrate, preferably mammals such as human patients and non-human primates, as well as other animals such as bovine, equine, canine, ovine, feline, murine and the like. In preferred embodiments, the subject, patient or individual is human. Accordingly, the term ‘subject’ or ‘patient’ as used herein means any mammalian patient or subject diagnosed with, predisposed to, or suspected of having a C9orf72-mediated disease. In embodiments, patients or subjects have, or are suspected of having C9orf72 hexanucleotide repeat expansions.

Patients or subjects with C9orf72-mediated diseases may have thousands of G₄C₂ repeats compared to median of two repeats in the general population. The number of repeats in healthy individuals is reported to be up to twenty-five or thirty GGGGCC hexanuceotide repeats (DeJesus-Hernandez et al., 2011). However, some studies have linked C9orf72 repeat expansions to neurological diseases such as Oculopharyngeal muscular dystrophy (OPMD), X-linked mental retardation or spinocerebellar ataxia 6 (SCA6) with as little as 11, 17 and 20 C9orf72 hexanucleotide repeats, respectively (van Blitterswijk et al. (2014). TMEM106B protects expansion C9ORF72 carriers against frontotemporal dementia. Acta Neuropathologica, 127(3): 397-406. doi: 10.1007/s00401-013-1240-4). The number of C9orf72 hexanucleotide repeats has been reported to be around four hundred to several thousand, although some ALS or FTD patients have shorter expansions around 45-80 repeats. Notably, there is an apparent gap between short pathogenic repeat sizes of 45 to 80 and long expansions from 400 to several thousand units. This is likely due to high genomic instability of the intermediate long repeats, which may have a tendency to either expand or contract. Interestingly, longer expansions may be correlated with an earlier onset of disease (Gijselinck et al. (2016). The C9orf72 repeat size correlates with onset age of disease, DNA methylation and transcriptional downregulation of the promoter. Molecular Psychiatry, 21(8): 1112-24. doi: 10.1038/mp.2015.159 and van Blitterswijk et al., 2014). Patients or subjects are typically heterozygous for the C9orf72 hexanucleotide expansion as this expansion results in an autosomal dominant phenotype. Therefore, the terms ‘subjects’ or ‘patients’, or ‘C9orf72-mediated disease’ refers to humans and/or non-human mammals with at least 15 G₄C₂ hexanucleotide repeats in one C9orf72 allele. In embodiments, the subject or patient has at least 15, 20, 25, 30, 35, 40, 50, 60, 70 or 80 G₄C₂ hexanucleotide repeats in at least one C9orf72 allele. More preferably, the subject or patient may have at least 100, 200, 300, 400, 500, 600,1000, 1500, 2000, 2500 or 3000 G₄C₂ hexanucleotide repeats in at least one C9orf72 allele. In one embodiment the terms ‘patients or subjects with hexanucleotide expansion repeats’ refers to mammals with at least 15, 20, 25, 30, 35, 40, 50, 60, 70, 80, 300, 400, 500, 600, 1000, 1500, 2000, 2500 or 3000 G₄C₂ hexanucleotide repeats in at least one C9orf72 allele. In another embodiment, the ‘subjects or patients with C9orf72 hexanucleotide expansion repeats’ can be grouped into patients with short expansions in at least one C9orf72 allele (around 15-80, 20-80, 25-80, 30-80, 40-80, or 45-80 G₄C₂ repeats) and patients with large expansions in at least one C9orf72 allele (at least 300, 400, 500, 600, 1000, 1500, 2000, 2500 or 3000 G₄C₂ repeats). In comparison, the term ‘healthy individual’ may refer to patients with up to 30, 25, 20, 15, 14, 13, 12, 11, 10, 9, 8, 7, 6, 5, 4, 3 or 2 G₄C₂ repeats in at least one C9orf72 allele.

Identification of C9orf72 repeat expansions may be established through standard clinical tests or assessments, such as genetic testing.

In some embodiments, the terms ‘subjects’ or ‘patients’ refers to any mammal with at least 15, 20, 25, 30, 35, 40, 50, 60, 70, 80, 300, 400, 500, 600, 1000, 1500, 2000, 2500 or 3000 G₄C₂ hexanucleotide repeats in at least one C9orf72 allele with or without a diagnosis of a C9orf72-mediated disease or symptoms. The terms ‘subject’ or ‘patient’ therefore refer to mammals diagnosed with a C9orf72-mediated disease, or any mammalian patient or subject with a risk of developing a C9orf72-mediated disease. Thus, in some embodiments, the present invention can be applied to a mammal who has at least 15, 20, 25, 30, 35, 40, 50, 60, 70, 80, 300, 400, 500, 600, 1000, 1500, 2000, 2500 or 3000 G₄C₂ hexanucleotide repeats in at least one C9orf72 allele, with or without symptoms or diagnosis of a C9orf72-mediated disease.

The compositions and methods described herein may, for example, be used to treat neurodegenerative diseases. Neurodegenerative diseases are characterized by the loss of specific neurons, and are complex, progressive, disabling, and often fatal. Neurodegenerative diseases can be divided into acute and chronic neurodegenerative diseases. The former mainly include stroke and brain injury, while the latter includes Amyotrophic Lateral Sclerosis (ALS), Parkinson's disease (PD), Huntington's Disease (HD), Alzheimer's disease (AD), and Frontotemporal Dementia (FTD) (Xu et al., 2021).

As used herein, the term ‘C9orf72-mediated disease’ is used in its broadest sense and generally refers to any ‘disease’, ‘condition’, ‘disorder’, or ‘pathology’ associated with C9orf72 hexanucleotide repeat expansions. As used herein, the terms ‘disease’, ‘condition’, ‘disorder’, or ‘pathology’ may be used interchangeably. Such diseases may include neurodegenerative diseases, including Amyotrophic Lateral Sclerosis (ALS), and Frontotemporal dementia (FTD). C9orf72 hexanucleotide repeat expansions may also be associated with sub-populations of Alzheimer's disease (AD), Huntington's disease (HD), and Parkinson's disease (PD) patients, although a causative role is yet to be established (Xu et al., 2021). C9orf72 expansions may also be associated with other neurological diseases, such as schizophrenia or bipolar disorder (Galimberti et al., 2014 and Meisler et al., 2013). In addition to FTD and ALS clinical indicators, C9orf72 FTD/ALS patients may suffer from neuropsychiatric symptoms and Parkinsonism (Cooper-Knock et al., 2014). C9orf72 patients have therefore also been diagnosed as Alzheimer, progressive supranuclear palsy, and Huntington disease patients further highlighting a clinical heterogeneity (Woollacott & Mead, 2014).

An example of a C9orf72-mediated disease is Amyotrophic Lateral Sclerosis (ALS). ALS is the most common adult-onset motor neuron disease and is fatal for most patients less than three years from when the first symptoms appear. ALS patients typically present with progressive muscular weakness, eventually leading to paralysis due to loss of upper and lower motor neurons (Ferrari et al., 2011; Ling et al., 2013). The age of onset is mainly between 30 and 60 years, affecting more men than women (Xu et al., 2021). Generally, it appears that the development of ALS in approximately 90-95% of patients is not associated with a clear family history of disease (sporadic ALS, sALS), with only 5-10% of patients displaying a clear family history (familial ALS, fALS). ALS has an annual incidence of 1-3 cases per 100,000 people. Until relatively recently, many familial cases of ALS had no known mutation. Mutations in several genes, including SOD1 (20-25%), TDP43/TARDBP, FUS, (TDP43/TARDBP and FUS together are 5%), ANG, ALS2, SETX, and VAPB, TBK1 genes, have since been found to cause familial ALS and contribute to the development of sporadic ALS (Baker et al., 2006;Hutton et al., 1998; Kabashi et al., 2008; Parkinson et al., 2006; Rosen et al., 1993). However, in 2011, it was discovered that the G₄C₂ hexanucleotide repeat expansion C9orf72 was the most common cause of familial ALS cases in Caucasian populations, accounting for 30 to 40% of familial ALS (DeJesus-Hernandez et al., 2011; Majounie et al., 2012; Renton et al., 2011). Therefore, the presently disclosed compositions, complexes, vectors and methods described herein can be used to the treatment and/or prevention of ALS.

Another C9orf72-mediated disease is frontotemporal dementia (FTD). FTD is a progressive disorder of the brain that can affect behaviour, language and movement. See, e.g., Benussi et al. (2015) Front Ag Neuro 7, art. 171. FTD patients present with gradual behavioural and cognitive impairments associated with neuronal atrophy of the frontal and temporal lobes (Ferrari et al., 2011; Ling et al., 2013). Mutations in MAPT, PGRN, VCP, and CHMP2B shown to cause FTD (Baker et al., 2006; Hutton et al., 1998; Kabashi et al., 2008; Parkinson et al., 2006; Rosen et al., 1993). In addition, it has been found that C9orf72 hexanucleotide expansions are the most common cause of familial FTD cases in Caucasian populations, accounting for 25% of familial FTD (DeJesus-Hernandez et al., 2011; Majounie et al., 2012; Renton et al., 2011). Therefore, the presently disclosed compositions, complexes, vectors and methods described herein can be used to the treatment and/or prevention of FTD.

The pathology associated with the C9orf72 hexanucleotide expansion appears to be related to expression of both sense and anti-sense transcripts and to the formation of unusual structures in the DNA and to some type of RNA-mediated toxicity (Taylor (2014) Nature 507:175). RNA transcripts of the expanded hexanucleotide repeat form nuclear foci in C9orf72 mutation patient cells and the RNAs can also undergo repeat-associate non-ATG-dependent translation, resulting in the production of three proteins that are prone to aggregation (Gendron et al. (2013). Antisense transcripts of the expanded C9ORF72 hexanucleotide repeat form nuclear RNA foci and undergo repeat-associated non-ATG translation in c9FTD/ALS. Acta

Neuropathologica, 126(6), 829-844. https://doi.org/10.1007/s00401-013-1192-8). Thus, the present invention described herein can be used for the treatment and/or prevention of FTD/ALS in a subject in need thereof.

Both loss and gain of function mechanisms have been proposed as pathogenic processes in C9orf72 FTD/ALS, with recent evidence suggesting these mechanisms act synergistically in disease pathogenesis (Zhu et al. 2020). Indeed, the majority of evidence suggests that C9orf72-related FTD/ALS is caused by a toxic gain of function (Mizielinska et al., 2014; Saberi et al., 2017; Stopford et al., 2017; Suzuki et al., 2018), however C9orf72 patients have a reduced expression of C9orf72 (˜50%) suggesting a potential loss of function contribution to disease pathogenesis (Jackson et al., 2020; Rizzu et al., 2016). C9orf72 is a suggested guanine exchange factor that has been implicated in the regulation of autophagy via the activation of Rab proteins (Iyer et al., 2018). C9orf72 FTD/ALS patients have reduced mRNA and protein levels of C9orf72 long and short isoforms due to the presence of the hexanucleotide expansion repeat (Rizzu et al., 2016). Loss of C9orf72 has been shown to impair autophagy, lysosomal biogenesis, and vesicular trafficking in cell models, with one report of C9orf7 2haploinsufficiency leading to neurodegeneration in human-derived cell models (Shi et al., 2018; Webster et al., 2016). Whilst C9orf72-knockout mice do not exhibit neurodegeneration or motor dysfunction, they do develop splenomegaly and exhibit peripheral and CNS immune cell deficits (Burberry et al., 2016; Koppers et al., 2015; O'Rourke et al., 2016; Sareen et al., 2013; Sudria-Lopez et al., 2016); however, it is not clear whether a ˜50% reduction in C9orf72, as is seen in patients, will lead to these pathologies. Perhaps more crucially, loss or reduction of C9orf72 function has been shown to exacerbate the gain of function mechanisms of the hexanucleotide expansion repeat with increased DPR accumulation, glial activation, and hippocampal neuron loss in a mouse model (Zhu et al., 2020). Therefore, an important part of any therapy should be to minimise any further reduction in C9orf72 expression.

There is strong evidence to suggest DPRs are toxic and a key pathogenic feature of the C9orf72 hexanucleotide repeat expansion with arginine-rich DPRs, poly-GR and poly-PR, but not repeat-containing RNA, associated with neurodegeneration in Drosophila and cellular models (Kanekura et al., 2016; Mizielinska et al., 2014; Tran et al., 2015; Wen et al., 2014). Additionally, poly-GR has been shown to correlate to neurodegeneration and co-localise and TDP-43 inclusions in C9orf72 patients (Saberi et al., 2018). Poly-GA has also been shown to be toxic in primary neurons, with a poly-GA expressing mouse model shown to develop neurodegeneration (Y. J. Zhang et al., 2016).

RNA foci formed of both the sense G₄C₂ and antisense C₄G₂ transcripts are also a key pathologic feature of C9orf72 hexanucleotide expansion repeat (Mizielinska et al., 2013).

While it is clear that the C9orf72 RNA foci sequester RNA binding proteins, there is evidence for and against the toxicity of the RNA foci (Moens et al., 2018; Swinnen et al., 2018; Xu et al., 2013).

Thus in some embodiments, the subject to be treated may be suffering from a neurodegenerative or other disorder involving the formation of one or more RNA foci. In some embodiments, a focus comprises at least one C9orf72 transcript. In some embodiments, the C9orf72 foci comprise transcripts comprising a hexanucleotide repeat expansion.

Clustered Regularly Interspaced Short Palindromic Repeats (CRISPR)-CRISPR-associated (Cas) (CRISPR-Cas) systems originate from Prokaryotes, where they serve primarily as a defensive mechanism against mobile genetic elements like phages and plasmids. CRISPR-Cas systems comprise Cas proteins and guide RNA which can be utilised in eukaryotic cells to induce degradation or modification of DNA or RNA sequences.

In brief, CRISPR-Cas systems use short (around <50 nucleotide) ‘guide’ RNA or DNA sequences that are complementary to the target RNA or DNA, respectively, and are therefore able to hybridise to the target sequence by Watson-Crick pairing. Upon hybridising to the target sequence, the guide/Cas effector enzyme complex undergoes a conformational change which activates the nucleolytic activity of the Cas effector protein which then cleaves the target sequence. Cleavage of the target RNA or DNA induces degradation or modification of the target sequence.

CRISPR-Cas systems are divided into two categories based on the proteins that form the Cas effector complex. Class 1 CRISPR-Cas effector complexes are assembled from a guide sequence and multiple protein subunits to form a complex, whereas Class 2 CRISPR-Cas effector complexes are assembled from a guide sequence and a single Cas protein. Classes 1 and 2 are then subdivided based on the Cas protein type; types I, III and IV for Class 1, and types II, V and VI for Class 2. The types can also be divided depending on the target sequence, whereas types I, II and V target DNA, type III targets DNA and RNA and type VI exclusively targets RNA.

Type VI CRISPR-Cas systems target RNA and use a single Cas effector protein called Cas13. Type VI Cas proteins include Cas13a (also referred to as C2c2 or VI-A), Cas13b (also referred to as C2c6 or VI-B), Cas13c (also referred to as C2c7 or VI-C) and Cas13d (also referred to as VI-D). Although the Type IV Cas13 proteins differ in size and sequence, they all share a common feature; the presence of two Higher Eukaryotes and Prokaryotes Nucleotide-binding (HEPN) domains. These domains are responsible for RNA-targeted ribonuclease activity which degrades target RNA (O′Connell. (2018). Molecular Mechanisms of RNA Targeting by Cas13-containing Type VI CRISPR-Cas Systems. Journal of Molecular Biology, 6-14. https://doi.org/10.1016/j.jmb.2018.06.029). HEPN domains are usually located close to different terminal ends of the Cas13 protein. Cas13 CRISPR-Cas systems function using one of the Cas13 effector protein subtypes (a, b, c or d) which forms a complex with a 60-66 nucleotide long guide RNA composed of a direct repeat sequence forming a single short hairpin loop (also referred to as a ‘stem loop’) followed by a 5′ or 3′ nucleotide spacer sequence which is complementary to the target RNA sequence. As described above, the guide RNA sequence hybridises with the target RNA, which induces a conformational change in the Cas13-gRNA complex, bringing the HEPN domains closer to each other and providing a single catalytic site for the Cas 13 effector protein to cleave the target RNA. Cas13 proteins also have a second type of ribonuclease activity which allows processing of a pre-gRNA array to form mature guide RNAs (pre-guide RNA) without additional domains, or other enzymes co-expressed (Konermann et al., 2018 and O′Connell, 2018) and in a HEPN domain-independent mechanism. When Cas13 effector proteins mature pre-guide RNAs they remove ˜8 nucleotides from the 3′ end of the pre-guide RNA. The 5′ 16 nucleotides of the pre-guide RNA, closest to the CRISPR direct-repeat, has been shown to be the most important region for guide specificity and efficiency (Zhang et al. (2018). Structural basis for the RNA-guided ribonuclease activity of CRISPR-Cas13d. BioRxiv, 175(1), 212-223.e17. https://doi.org/10.1101/314401). However, it has been shown that gRNA maturation is not necessary for type VI effector protein activity, and even unprocessed pre-gRNA is sufficient for recognition of targeted RNA (East-Seletsky et al., 2017).

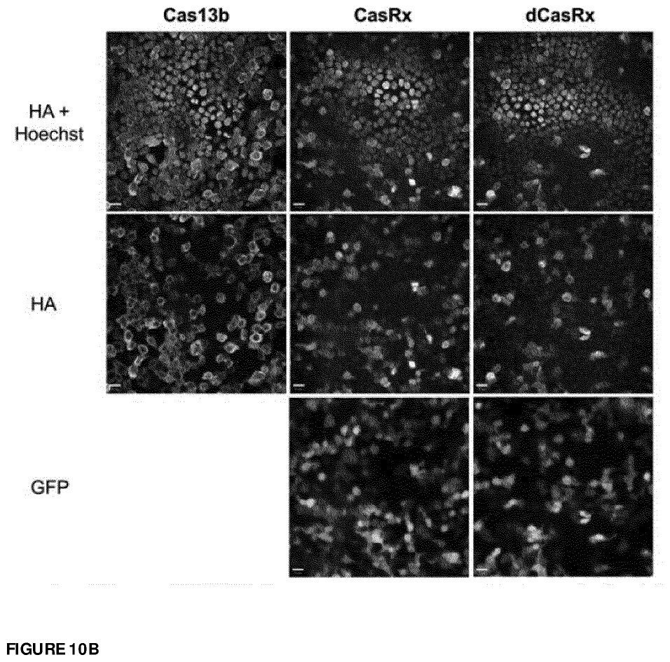

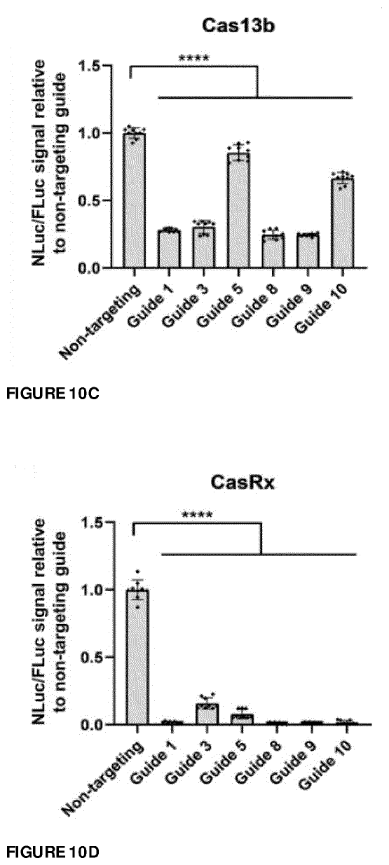

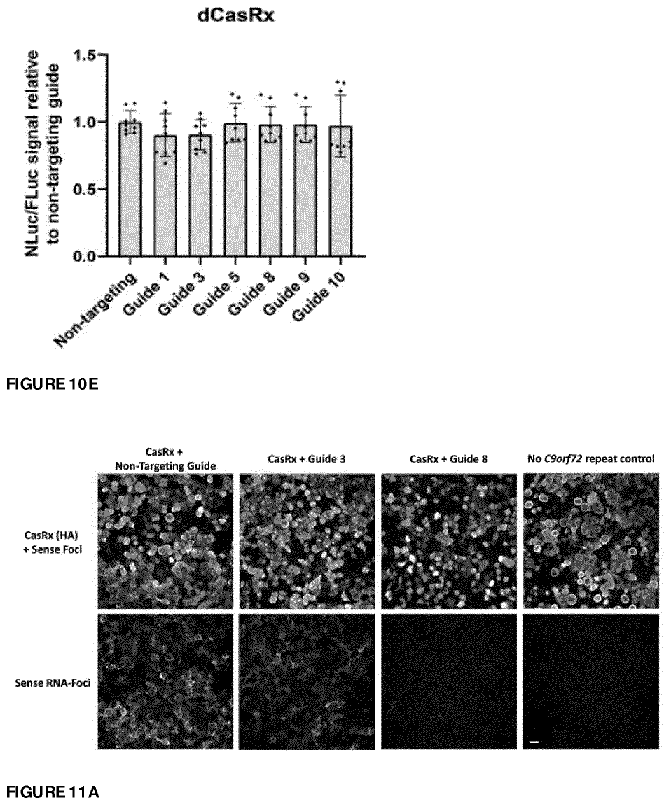

Whilst type VI CRISPR-Cas systems represent a promising new therapeutic avenue for RNA-related disorders (Abudayyeh et al. (2017). RNA targeting with CRISPR-Cas13. Nature, 550(7675), 280-284. https://doi.org/10.1038/nature24049, Cox et al. (2017). RNA editing with CRISPR-Cas13 David. Science, 1027(November), 1019-1027. https://doi.org/10.1126/science.aaq0180, Konermann et al., 2018; Zhang et al., 2018), their size (around 1,200 amino acids (aa)) makes them slightly too large to package into adeno-associated virus (AAV) for primary cell and in vivo delivery. However, Cas13d is around 930 amino acids, and is the smallest class 2 CRISPR effector characterised in mammalian cells. Cas13d has also been optimised for efficient transcript knockdown by addition of N-terminal and C-terminal nuclear localisation sequences (NLS), and this variant has been termed CasRx (FIG. 10A; Konermann et al., 2018). Use of a small Cas effector protein allows Cas13d/CasRx effector domain fusions to be paired with a CRISPR array encoding multiple guide RNAs while remaining under the packaging size limit of the versatile AAV delivery vehicle.

As used herein, the terms ‘Cas protein’, ‘Cas effector’ or ‘effector protein’ may be used interchangeably to refer to the CRISPR-associated (Cas) proteins. Cas proteins are nucleases which play an effector role in CRISPR-Cas systems. In embodiments, the CRISPR-Cas effector protein is a class 2 Cas protein. In embodiments, the CRISPR-Cas effector is a Type IV Cas protein. In embodiments, the CRISPR-Cas effector protein may be a Cas13, such as Cas13a, Cas13b, Cas13c or Cas13d. In preferred embodiments, the CRISPR-Cas effector protein is Cas13b or Cas13d. In more preferred embodiments, the Cas13 protein is Cas 13d or CasRx. As used herein, “CasRx/Cas13d” means CasRx and/or Cas13d.

Exemplary Cas sequences (e.g. CasRx/Cas13d protein sequences and nucleic acid sequences encoding such proteins) are disclosed in e.g. WO 2019/236982 (see e.g. SEQ ID NO:s 45-51, 54, 57, 61, 67, 69, 71-73, 84-115 thereof) and WO 2020/214830, the contents of which are incorporated herein by reference. Further suitable sequences are disclosed or cited in e.g. Konermann et al., Cell. 2018 Apr. 19; 173(3): 665-676.e14 and Yan et al., Mol Cell. 2018 Apr. 19; 70(2): 327-339.e5. Thus in specific embodiments, the composition comprises a nucleic acid sequence encoding a CasRx/Cas13d polypeptide complex as defined in one of the above documents, or the complex comprises a CasRx/Cas13d polypeptide having a sequence as defined therein (e.g. in any of SEQ ID NO:s 45-51, 54, 57, 61, 67, 69, 71-73, 84-115 of WO 2019/236982). In embodiments, the composition, complex, or vector comprises a nucleic acid sequence encoding a CasRx as defined in Konermann et al., Cell. 2018 Apr. 19; 173(3): 665-676.e14. In embodiments, the Cas13d is encoded by a polypeptide sequence SEQ ID NO: 64. In embodiments, the CasRx is encoded by a polypeptide sequence comprising SEQ ID NO: 65.

As used herein, the ‘target RNA’, ‘target sequence’, ‘target RNA transcript’ or ‘target transcript’ are used interchangeably to refer to any endogenous or exogenous, sense or antisense RNA transcript of the C9orf72 gene (SEQ ID NO: 56). In preferred embodiments, the C9orf72 ‘target RNA’ comprises a hexanucleotide repeat expansion. In embodiments, the target RNA is a messenger RNA (mRNA) or precursor mRNA (pre-mRNA).

As used herein, the terms ‘guide’ or ‘spacer’ are used interchangeably to refer to any polynucleotide sequence having sufficient complementarity with the target RNA sequence. In embodiments, the spacer sequence is between 15-40, 20-40, 15-35, 20-35, 15-30, 20-30 or 22-30 nucleotides in length. In preferred embodiments, the guide sequence is 20-30 nucleotides long. In embodiments, the spacer sequence is equal to or more than 15, 18, 20, 21, 22, 23, 24, 25, 26, 27, 28, 29, 30, 31, 32, 33, 34, 35, 36, 37, 38, 39 or 40 nucleotides in length. In some embodiments, the degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, such as Clustal W or BLAST.

In embodiments, the spacer sequence targets a sequence in a sense C9orf72 RNA transcript corresponding to base pairs 150-400 of the C9orf72 gene (SEQ ID NO: 56). In preferred embodiments, the spacer sequence targets a sequence in a sense C9orf72 RNA transcript corresponding to base pairs 150-350, 200-350, or 200-320 of the C9orf72 gene (SEQ ID NO: 56). In preferred embodiments, the spacer sequence targets a sequence in a sense C9orf72RNA transcript corresponding to base pairs 201-320 (SEQ ID NO: 60) of the C9orf72 gene (SEQ ID NO: 56). In specific embodiments, the spacer sequence targets a sequence in a sense C9orf72 RNA transcript corresponding to base pairs selected from: 201-230, 211-240, 221-250, 231-260, 241-270, 251-280, 261-290 271-300, 281-310, or 291-320 of the C9orf72 gene (SEQ ID NO: 56). In preferred embodiments, the spacer sequence targets a sequence in a sense C9orf72 RNA transcript corresponding to base pairs selected from 201-230, 271-300, 281-310, or 291-320 of the C9orf72 gene (SEQ ID NO: 56). In embodiments, the spacer sequence has equal to or more than 80, 85, 90, 91, 92, 93, 94, 95, 96, 97, 98, 99 or 100% sequence identity to a sense RNA transcript corresponding to base 150-400, 150-350, 200-350, or 200-320 of the C9orf72 gene (SEQ ID NO: 56). In preferred embodiments, the spacer sequence targeting the C9orf72 sense transcript comprises, consists or consists essentially of SEQ ID NOs: 1, 4, 7, 10, 13, 16, 19, 22, 25 or 28. In preferred embodiments, the spacer sequence targeting the C9orf72 sense transcript comprises, consists or consists essentially of SEQ ID NOs: 1, 22, 25 or 28. In some embodiments, the spacer sequence targeting the C9orf72 sense transcript has equal to or more than 80, 85, 90, 91, 92, 93, 94, 95, 96, 97, 98, 99 or 100% sequence identity to SEQ ID NOs: 1, 4, 7, 10, 13, 16, 19, 22, 25 or 28.

It will be appreciated that the sense C9orf72 RNA transcript comprises an RNA sequence whereas the C9orf72 gene (SEQ ID NO: 56) comprises a DNA sequence. Therefore, it will be understood with respect to the above embodiments that the sense transcript comprises an RNA sequence corresponding to the sense strand of the DNA C9orf72 gene at particular regions of the C9orf72 gene as defined in SEQ ID NO: 56. It will also be understood that by “corresponding to” it is meant that the spacer sequence binds specifically to, or is complementary to, a sequence within the specified region of the sense strand of the C9orf72 gene, e.g. within base pairs 150-400, 150-350, 200-350 or 200-320 of SEQ ID NO: 56.

In embodiments, the spacer sequence targets a sequence in an anti-sense C9orf72 RNA transcript complementary to base pairs 350-700 of the C9orf72 gene (SEQ ID NO: 56). In preferred embodiments, the spacer sequence targets a sequence in an anti-sense C9orf72 RNA transcript complementary to base pairs 350-650, 400-700, 350-600, 400-650, 400-600, or 410-575 of the C9orf72 gene (SEQ ID NO: 56). In preferred embodiments, the spacer sequence targets a sequence in an anti-sense C9orf72 RNA transcript complementary to base pairs 418-574 (SEQ ID NO: 61) of the C9orf72 gene (SEQ ID NO: 56). In specific embodiments, the spacer sequence targets a sequence in an anti-sense C9orf72 RNA transcript complementary to base pairs selected from: 418-447, 398-427, 539-567, 478-507, or 545-574 of the C9orf72 gene (SEQ ID NO: 56). In preferred embodiments, the spacer sequence targets a sequence in an anti-sense C9orf72 RNA transcript complementary to base pairs selected from: 418-447, 539-567, 478-597 or 545-574 of the C9orf72 gene (SEQ ID NO: 56). In more preferred embodiments, the spacer sequence targets a sequence in an anti-sense C9orf72 RNA transcript complementary to base pairs 545-574 of the C9orf72 gene (SEQ ID NO: 56). In embodiments, the spacer sequence has equal to or more than 80, 85, 90, 91, 92, 93, 94, 95, 96, 97, 98, 99 or 100% sequence identity to an antisense RNA transcript complementary to base pairs 350-700, 350-650, 400-700, 350-600, 400-650, 400-600, or 410-575 of the C9orf72 gene (SEQ ID NO: 56). In preferred embodiments, the spacer sequence targeting the C9orf72 antisense transcript comprises, consists or consists essentially of SEQ ID NOs: 31, 34, 37, 40 or 43. In preferred embodiments, the spacer sequence targeting the C9orf72 antisense transcript comprises, consists or consists essentially of SEQ ID NOs: 31, 37, 40 or 43. In some embodiments, the spacer sequence targeting the C9orf72 antisense transcript has equal to or more than 80, 85, 90, 91, 92, 93, 94, 95, 96, 97, 98, 99 or 100% sequence identity to SEQ ID NOs: 31, 34, 37, 40 or 43.

It will be appreciated that the antisense C9orf72 RNA transcript comprises an RNA sequence complementary to the DNA sequence of the C9orf72 gene as defined in SEQ ID NO: 56. Therefore, it will be understood with respect to the above embodiments that the antisense transcript comprises an RNA sequence that comprises nucleotide residues complementary to the nucleotide residues in SEQ ID NO:56, and that the RNA sequence reads, in a 5′ to 3′ direction, in the opposite direction to the DNA sequence in SEQ ID NO:56. It will also be appreciated that by “complementary to” it is meant that the spacer sequence binds specifically to, or is complementary to, a sequence in the antisense strand of C9orf72 gene that is complementary to or within the specified region in the sense strand (SEQ ID NO:56). For instance, the spacer sequence may comprise an RNA sequence corresponding to a DNA sequence within residues 350-700, 350-650, 400-700, 350-600, 400-650, 400-600, or 410-575 of the sense strand of the C9orf72 gene (SEQ ID NO: 56); or the spacer sequence may be complementary to a sequence in the antisense strand of the C9orf72 gene that is complementary to a sequence within residues 350-700, 350-650, 400-700, 350-600, 400-650, 400-600, or 410-575 of the sense strand (SEQ ID NO:56).

As used herein, the term ‘direct repeat’ refers to the nucleotide sequence of the guide RNA which forms a single short hairpin loop. In embodiments, the pre-gRNA direct repeat has SEQ ID NO: 46 (CAAGTAAACCCCTACCAACTGGTCGGGGTTTGAAAC). In embodiments, the mature gRNA direct repeat sequence is SEQ ID NO: 47 (AACCCCTACCAACTGGTCGGGGTTTGAAAC).

As used herein, the term ‘non-targeting guide’, ‘guide NT’, ‘NT guide’, ‘non-targeting control guide’ or ‘non-targeting control gRNA’ are used interchangeably to refer to a nucleotide comprising a guide or spacer sequence that does not target a C9orf72 transcript. In one embodiment, the term non-targeting guide is used to refer to any RNA comprising a sequence comprising, consisting, or consisting essentially of SEQ ID NO: 77.

As used herein, the terms ‘pre-guide+spacer’, ‘pre-gRNA’, ‘pre-gRNA+spacer’ or ‘pre-guide RNA’ are used interchangeably to refer to the immature pre-gRNA sequence comprising the pre-gRNA direct repeat with SEQ ID NO: 46 followed by a ‘spacer’ sequence. The term pre-gRNA therefore refers to the sequence as found in a plasmid or vector, or as found in the cells without processing by a Cas13 effector enzyme. In embodiments, the pre-gRNA targets a sequence in a sense C9orf72 RNA transcript corresponding to base pairs 150-400 of the C9orf72 gene (SEQ ID NO: 56). In embodiments, the antisense pre-gRNA targets a sequence in an antisense C9orf72 RNA transcript corresponding to base pairs 350-700 of the C9orf72 gene (SEQ ID NO: 56). In embodiments, pre-gRNA targeting the sense C9orf72 RNA transcript comprises, consists or consists essentially of SEQ ID NOs: 2, 5, 8 11, 14, 17, 20, 23, 26 or 29. In embodiments, pre-gRNA targeting the antisense C9orf72 RNA transcript comprises, consists or consists essentially of SEQ ID NOs: 32, 35, 38, 41 or 44. In preferred embodiments, the pre-gRNA has equal to or more than 80, 85, 90, 91, 92, 93, 94, 95, 96, 97, 98, 99 or 100% sequence identity to SEQ ID NOs: 2, 5, 8, 11, 14, 17, 20, 23, 26, 29, 32, 35, 38, 41 or 44.

In some embodiments the pre-gRNA form a ‘guide array’ comprising two or more pre-gRNA sequences. In some embodiments the pre-gRNA in a guide array are arranged consecutively. In other embodiments the pre-gRNA in a guide array are separated by at least 1, 2, 3, 5, 10, or 20 nucleotides. In some embodiments, the guide array comprises two or more pre-gRNA that target a sequence in a sense C9orf72 RNA transcript corresponding to base pairs 150-400 of the C9orf72 gene (SEQ ID NO: 56). In some embodiments, the guide array comprises two or more pre-gRNA that target a sequence in an antisense C9orf72 RNA transcript corresponding to base pairs 350-700 of the C9orf72 gene (SEQ ID NO: 56). In preferred embodiments the guide array comprises one or more pre-gRNA targeting the sense C9orf72 RNA transcript, and one or more pre-gRNA targeting the antisense C9orf72 RNA transcript. In preferred embodiments, the one or more pre-gRNA in a guide array comprise spacer sequences selected from the list comprising SEQ ID NOs: 1, 4, 7, 10, 13, 16, 19, 22, 25, 28, 31, 34, 37, 40, 43, or any combination thereof. In embodiments, the guide array comprises one or more pre-gRNA selected from the list comprising SEQ ID NOs: 2, 5, 8, 11 14, 17, 20, 23, 26, 29, 32, 35, 38, 41, 44 or any combination thereof. In preferred embodiments, the guide array comprises a first pre-gRNA targeting a sequence in a sense C9orf72 RNA transcript, and a second pre-gRNA targeting a sequence in an antisense C9orf72 RNA transcript. In preferred embodiments, the guide array comprises SEQ ID NOs: 29 and 44.

As used herein, the terms ‘mature guides’, ‘mature guide RNA’, or ‘mature gRNA’, are used interchangeably to refer to the mature gRNA sequence comprising the mature gRNA direct repeat sequence (SEQ ID NO: 47) followed by a spacer sequence. The mature gRNA therefore reflects the gRNA sequence as found in the cell upon processing by the Cas13 effector enzyme. In embodiments, the mature gRNA targeting the sense C9orf72 RNA transcript comprises, consists or consists essentially of SEQ ID NOs: 3, 6, 9, 12, 15, 18, 21, 24, 27 or 30. In embodiments, the mature gRNA targeting the antisense C9orf72 RNA transcript comprises, consists or consists essentially of SEQ ID NOs: 33, 36, 39, 42 or 45. In preferred embodiments, the mature gRNA has equal to or more than 80, 85, 90, 91, 92, 93, 94, 95, 96, 97, 98, 99 or 100% sequence identity to SEQ ID NOs: 3, 6, 9, 12, 15, 18, 21, 24, 27, 30, 33, 36, 39, 42 or 45.

A person skilled in the art will appreciate that the disclosed guide sequences (including the spacer, pre-gRNA+spacer and mature gRNA+spacer sequences) may be used in combination. In particular, a skilled person will appreciate that it is possible to administer a first guide that targets a sequence in a sense C9orf72 RNA transcript, and a second guide that targets a sequence in an antisense C9orf72 RNA transcript.

In some embodiments, the pre-gRNA or mature gRNA comprises one or more point mutations that improve expression levels of the pre-gRNAs or mature gRNAs via removal of partial or full transcription termination sequences or sequences that destabilize pre-gRNA or mature gRNAs after transcription via action of transacting nucleases. In some embodiments, the pre-gRNA or mature gRNA comprises an alteration at the 5′ end which stabilizes said pre-gRNA or mature gRNA against degradation. In some embodiments, the pre-gRNA or mature gRNA comprises an alteration at the 5′ end which improves RNA targeting. In some embodiments, the alteration at the 5′ end of said pre-gRNA or mature gRNA is selected from the group consisting of 2′0-methyl, phosphorothioates, and thiophosphonoacetate linkages and bases. In some embodiments, the pre-gRNA or mature gRNA comprises 2′-fluorine, 2′0-methyl, and/or 2′-methoxyethyl base modifications in the spacer or scaffold region of the pre-gRNA or mature gRNA to improve target recognition or reduce nuclease activity on the pre-gRNA or mature gRNA. In some embodiments, the pre-gRNA or mature gRNA comprises one or more methylphosphonate, thiophosponoaceteate, or phosphorothioate linkages that reduce nuclease activity on the target RNA.

As used herein, the term ‘guide RNA’ or ‘gRNA’ refers collectively to a ‘guide’, ‘pre-gRNA’, mature ‘gRNA’ or ‘pre-gRNA array’.

The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence. Host cells can include cells provided with vectors comprising the target sequence such as through transfection, or patient-derived iPSCs which endogenously express the target sequence. This can then be followed by an assessment of preferential cleavage of the target sequence.

As used herein, the terms ‘CRISPR-Cas system’, or ‘CRISPR system’ are used interchangeably to refer collectively to the combination of a guide RNA and a CRISPR-Cas effector protein in a cell. In embodiments, the term ‘CRISPR system’ refers to the use of one or more gRNAs and a Cas effector protein. In preferred embodiments, the Cas effector protein is Cas13a, Cas13b, Cas13c, Cas13d or CasRx, and the one or more gRNAs is complementary to a C9orf72 sense or antisense RNA transcript. In preferred embodiments, the Cas effector protein is Cas13d or CasRx, and the one or more gRNAs targets a C9orf72 sense or antisense RNA transcript. In embodiments the CRISPR system comprises a Cas13d or CasRx effector protein in combination with one or more gRNAs targeting a sequence in the sense C9orf72 RNA transcript corresponding to base pairs 150-400 of the C9orf72 gene (SEQ ID NO: 56). In embodiments the CRISPR system comprises a Cas13d or CasRx effector protein in combination with one or more gRNAs targeting a sequence in a sense C9orf72 RNA transcript corresponding to base pairs 150-350, 200-350, or 200-320 of the C9orf72 gene (SEQ ID NO: 56). In embodiments the CRISPR system comprises a Cas13d or CasRx effector protein in combination with one or more gRNAs targeting a sequence in the antisense C9orf72 RNA transcript corresponding to base pairs 350-700 of the C9orf72 gene (SEQ ID NO: 56). In embodiments the CRISPR system comprises a Cas13d or CasRx effector protein in combination with one or more gRNAs targeting a sequence in an antisense C9orf72 RNA transcript corresponding to base pairs 350-650, 400-700, 350-600, 400-650, 400-600, or 410-575 of the C9orf72 gene (SEQ ID NO: 56). In embodiments, the CRISPR system comprises a Cas13d or CasRx effector protein in combination with one or more pre-gRNAs targeting a sequence in the sense C9orf72 RNA transcript corresponding to base pairs 150-400 and/or the antisense C9orf72 transcript corresponding to base pairs 350-700 of the C9orf72 gene (SEQ ID NO: 56), or a combination thereof. In preferred embodiments, the CRISPR system comprises a Cas13d or CasRx effector protein in combination with one or more gRNAs comprising, consisting or consisting essentially of spacer sequences with SEQ ID NOs: 1, 4, 7,10, 13, 16, 19, 22, 25, 28, 31, 34, 37, 40 or 43, or a combination thereof. In embodiments, the CRISPR system comprises a Cas13d or CasRx effector protein in combination with one or more pre-gRNAs comprising, consisting or consisting essentially of SEQ ID NOs: 2, 5, 8, 11, 14, 17, 20, 23, 26, 29, 32, 35, 38, 41 or 44, or a combination thereof. In embodiments, the CRISPR system comprises a Cas13d or CasRx effector protein in combination with one or more pre-gRNA array(s) comprising, consisting or consisting essentially of two or more pre-gRNAs selected from SEQ ID NOs: 2, 5, 8, 11, 14, 17, 20, 23, 26, 29, 32, 35, 38, 41 or 44. In embodiments, the CRISPR system comprises a Cas13d or CasRx effector protein in combination with one or more mature gRNAs comprising, consisting or consisting essentially of SEQ ID NOs: 3, 6, 9, 12, 15, 18, 21, 24, 27, 30 33, 36, 39, 42 or 45, or a combination thereof.

As used herein, the terms ‘CRISPR-Cas effector complex’, or ‘effector complex’ are used interchangeably to refer to the guide sequence and the effector protein in a complex. In embodiments, the effector complex comprises a Cas13 effector protein (i.e., Cas13a, Cas13b, Cas13c or Cas13d) in combination with one or more gRNAs targeting a C9orf72 sense or antisense RNA transcript. In preferred embodiments the effector complex comprises a Cas13d or CasRx effector protein in combination with one or more gRNAs targeting a C9orf72 sense or antisense RNA transcript. In embodiments the effector complex comprises a Cas13d or CasRx effector protein in combination with one or more gRNAs targeting a sequence in the sense C9orf72 RNA transcript corresponding to base pairs 150-400 of the C9orf72 gene (SEQ ID NO: 56). In embodiments the effector complex comprises a Cas13d or CasRx effector protein in combination with one or more gRNAs targeting a sequence in a sense C9orf72 RNA transcript corresponding to base pairs 150-350, 200-350, or 200-320 of the C9orf72 gene (SEQ ID NO: 56). In embodiments the effector complex comprises a Cas13d or CasRx effector protein in combination with one or more gRNAs targeting a sequence in the antisense C9orf72 RNA transcript corresponding to base pairs 350-700 of the C9orf72 gene (SEQ ID NO: 56). In embodiments the effector complex comprises a Cas13d or CasRx effector protein in combination with one or more gRNAs targeting a sequence in an antisense C9orf72 RNA transcript corresponding to base pairs 350-650, 400-700, 350-600, 400-650, 400-600, or 410-575 of the C9orf72 gene (SEQ ID NO: 56). In embodiments, the effector complex comprises a Cas13d or CasRx effector protein in combination with one or more pre-gRNAs targeting a sequence in the sense C9orf72 RNA transcript corresponding to base pairs 150-400 and/or the antisense C9orf72 transcript corresponding to base pairs 350-700 of the C9orf72 gene (SEQ ID NO: 56), or a combination thereof. In preferred embodiments, the effector complex comprises a Cas13d or CasRx effector protein in combination with one or more gRNAs comprising, consisting or consisting essentially of spacer SEQ ID NOs: 1, 4, 7, 10, 13, 16, 19, 22, 25, 28, 31, 34, 37, 40 or 43, or a combination thereof. In embodiments, the effector complex comprises a Cas13d or CasRx effector protein in combination with one or more pre-gRNA comprising, consisting or consisting essentially of SEQ ID NOs: 2, 5, 8, 11, 14, 17, 20, 23, 26, 29, 32, 35, 38, 41 or 44, or a combination thereof. In embodiments, the effector complex comprises a Cas13d or CasRx effector protein in combination with one or more pre-gRNA array(s) comprising, consisting or consisting essentially of two or more pre-gRNAs selected from SEQ ID NOs: 2, 5, 8, 11, 14, 17, 20, 23, 26, 29, 32, 35, 38, 41 or 44, or a combination thereof. In embodiments, the effector complex comprises a Cas13d or CasRx effector protein in combination with one or more mature gRNA comprising, consisting or consisting essentially of SEQ ID NOs: 3, 6, 9, 12, 15, 18, 21, 24, 27, 30 33, 36, 39, 42 or 45, or a combination thereof.

As used herein, the terms ‘degrade’, or ‘cleave’ are typically used interchangeably to refer to formation of at least one break in the RNA strand. Typically, formation of a ‘CRISPR-Cas effector complex’ results in cleavage of RNA strand(s) in or near (e.g., within 1, 2, 3, 4, 5, 6, 7, 8, 9, 10, 20, 50, or more base pairs from) the target sequence. In embodiments, the CRISPR-Cas effector complex cleaves C9orf72 mRNA or pre-mRNA. In preferred embodiments, the RNA-targeting complex cleaves C9orf72 sense and/or antisense RNA containing C9orf72 hexanucleotide repeat expansions.

Variants of the amino acid and nucleotide sequences described herein may also be used in the present invention. For instance, in specific embodiments, the present invention may involve variants of e.g. Cas13s, CasRxs, guide RNAs, spacer sequences, direct repeat sequences, target sequences and C9orf72 gene sequences. Typically such variants have a high degree of sequence identity with one of the sequences specified herein.

The similarity between amino acid or nucleotide sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of the amino acid or nucleotide sequence will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of the specific sequences described herein (e.g. a guide sequence or any one of SEQ ID NO:s 1 to 56) typically have at least about 75%, for example at least about 80%, 90%, 95%, 96%, 97%, 98% or 99% sequence identity with the original sequence (e.g. a sequence defined herein), for example counted over at least 20, 50, 100, 200 or 500 nucleotide or amino acid residues or over the full length alignment using the NCBI Blast 2.0, gapped blastp set to default parameters. For comparisons of nucleotide or amino acid sequences of greater than about 30 nucleotides or amino acids, the Blast 2 sequences function is employed using the default BLOSUM62 matrix set to default parameters, (gap existence cost of 11, and a per residue gap cost of 1). When aligning short oligonucleotides or peptides (fewer than around 30 residues), the alignment should be performed using the Blast 2 sequences function, employing the PAM30 matrix set to default parameters (open gap 9, extension gap 1 penalties). Polynucleotides or polypeptides with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 residues, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

“Binding” as used herein can refer to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions are generally characterized by a dissociation constant (Kd) of less than 10⁻³ M, less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M or less than 10⁻¹⁵ M. Kd is dependent on environmental conditions, e.g., pH and temperature, as is known by those in the art. “Affinity” can refer to the strength of binding, and increased binding affinity is correlated with a lower Kd. Thus the terms “binds to”, “associates with” and “forms a complex with” may be used interchangeably herein. For instance, the guide RNA may bind to, associate with or form a complex with the CasRx/Cas13d polypeptide.

The terms “hybridizing” or “hybridize” can refer to the pairing of substantially complementary or complementary nucleic acid sequences within two different molecules. Pairing can be achieved by any process in which a nucleic acid sequence joins with a partially, substantially or fully complementary sequence through base pairing to form a hybridization complex. For purposes of hybndization, two nucleic acid sequences or segments of sequences are “substantially complementary” if at least 80% of their individual bases are complementary to one another. Two nucleic acid sequences or segments of sequences are “partially complementary” if at least 50% of their individual bases are complementary to one another.

As used herein, “complementary” can mean that two nucleic acid sequences have at least 50% sequence identity. Preferably, the two nucleic acid sequences have at least 60%, 70%, 80%, 90%, 95%, 96%, 97%, 98%, 99%, or 100% of sequence identity. “Complementary” also means that two nucleic acid sequences can hybridize under low, middle, and/or high stringency condition(s).

As used herein, “complementary” preferably means e.g. that two nucleic acid sequences have at least 90% sequence identity. Preferably, the two nucleic acid sequences have at least 95%, 96%, 97%, 98%, 99%, or 100% of sequence identity. “Complementary” preferably means that two nucleic acid sequences can hybridize under high stringency condition(s).

Low stringency hybridization refers to conditions equivalent to hybridization in 10% formamide, 5× Denhardt's solution, 6× SSPE, 0.2% SDS at 22° C., followed by washing in 1× SSPE, 0.2% SDS, at 37° C. Denhardt's solution contains 1% Ficoll, 1% polyvinylpyrolidone, and 1% bovine serum albumin (BSA). 20× SSPE (sodium chloride, sodium phosphate, ethylene diamide tetraacetic acid (EDTA)) contains 3M sodium chloride, 0.2M sodium phosphate, and 0.025 M (EDTA). Other suitable moderate stringency and high stringency hybridization buffers and conditions are well known to those of skill in the art.

As used herein, the term ‘vector’ refers to any construct capable of delivery and optionally expressing any of the polynucleotides, polypeptides, nucleases, pre-gRNA, mature-gRNA, pre-gRNA arrays or guide sequences as described herein to a host cell, patient or subject. Examples of vectors include plasmids (also referred to as ‘expression constructs’), RNA expression vectors, nucleic acids complexed with a delivery vehicle such as liposome or poloxamer, viral vectors (including retroviral vectors, adenovirus vectors, poxvirus vectors, lentiviral vectors, herpesvirus vectors or adeno-associated virus vectors), or phage (bacteria) vectors. Viral vectors may be either replication competent or replication defective vectors. In embodiments, the Cas effector protein and the one or more guide, mature gRNA, pre-gRNA, or pre-gRNA array are carried on the same vector. In embodiments, the Cas effector protein and the one or more guide, mature gRNA, pre-gRNA or pre-gRNA arrays are carried on different vectors. In preferred embodiments, the vector is an adeno-associated virus (AAV). Even more preferably, the vector is a recombinant AAV (rAAV).

AAV belongs to the genus Dependoparvovirus within the family Parvoviridae. The AAV life cycle is dependent on the presence of a helper virus, such as adeno viruses. AAVs are composed of an icosahedral protein capsid ˜26 nm in diameter and a single-stranded DNA genome of ˜4.7 kb which is flanked by inverted terminal repeats (ITRs) that are required for genome replication and packaging. rAAVs are composed of the same capsid sequence and structure as found in wild-type AAVs. However, rAAVs encapsidate genomes that are devoid of all wild type AAV protein-coding sequences which are instead replaced with therapeutic gene expression cassettes (also referred to as a transgene). The only sequences of viral origin in rAAVs are the ITRs, which are needed to guide genome replication and packaging during vector production. The complete removal of viral coding sequences maximizes the packaging capacity of rAAVs and contributes to their low immunogenicity and cytotoxicity when delivered in vivo. Therefore, as used herein, the term ‘AAV vector’ refers to a vector comprising one or more polynucleotides of interest (or transgenes) that are flanked by AAV ITR sequences. There are several identified AAV serotypes, and different serotypes interact with serum proteins in different ways. Serology of AAVs is an important functional characteristic for cell specific transduction efficiency within the CNS. In embodiments the AAV serotype is: AAV1, AAV2, AAV3, AAV5, AAV6, AAV7, AAV8 and AAV9. In preferred embodiments, the AAV serotype is: AAV1, AAV2, AAV4, AAV5, AAV8 or AAV9. AAV hybrid serotypes or pseudo-serotypes have been created by viral engineering, which are constructed with integrated genome containing (cis-acting) inverted terminal repeats (ITR) of AAV2 and capsid genes of other serotypes for increased viral specificity and transduction. Therefore, in embodiments, the AAV vector is a hybrid serotype. In preferred embodiments the AAV is AAV-PHP.B,-PHP.eB or PHP.S. Whereas AAV-PHP.B transduces the majority of neurons and astrocytes across many regions of the central nervous system, AAV-PHP.eB has been found to reduce the required viral load.

As used herein, the term ‘promoter’ refers to a nucleic acid that serves to control the transcription of one or more polynucleotides, located upstream from the polynucleotide(s) sequence. In some embodiments, the promoter sequence is expressed in many tissue/cell types (i.e., ubiquitous), while in other embodiments, the promoter is tissue or cell specific. In preferred embodiments, the promoter sequence is specific for neuronal cells. In some embodiments the promoter may be constitutive or inducible. Non-limiting examples of ubiquitous promoters include CMV, CAG, Ube, human beta-actin, Ubc, SV40 or EF1a. Non-limiting examples of neuron-specific promoters include neuron-specific enolase (NSE), Synapsin, calcium/calmodulin-dependent protein kinase II, tubulin alpha I, and MECPs. In other embodiments, the promoter sequence is specific for muscle cells, such as muscle creatine kinase (MCK). Non-limiting examples of promoters suitable for use in plasmid vectors to drive expression of guides, pre-gRNA or mature gRNA include RNA polymerase III promoters. Examples of RNA polymerase III promoters include U6 and H1.

As used herein, the term ‘transduction’ refers to the process by which a sequence of foreign nucleotides is introduced into the cell by a virus.

As used herein, the term ‘transfection’ refers to the introduction of DNA into the recipient eukaryotic cells.

In some embodiments, the CRISPR-Cas complex or CRISPR system is associated with, or comprise, a detectable agent, such as a reporter agent or detectable epitope tags. Suitable reporter agents include, but are not limited to: proteins that mediate antibiotic resistance (e.g., ampicillin resistance, neomycin resistance, G418 resistance, or puromycin resistance), coloured, fluorescent or luminescent proteins (e.g., a green fluorescent protein (GFP), an enhanced GFP (eGFP), a blue fluorescent protein or its derivatives (EBFP, EBFP2, Azurite, mKalamal), a cyan fluorescent protein or its derivatives (ECFP, Cerulean, CyPet, m Turquoise2), a yellow fluorescent protein and its derivatives (YFP, Citrine, Venus, YPet), UnaG, dsRed, eqFP61 1, Dronpa, TagRFPs, KFP, EosFP, Dendra, IrisFP, mcherry, or luciferase), or proteins which mediate enhanced cell growth and/or gene amplification (e.g., dihydrofolate reductase). Suitable epitope tags may include one or more copies of the FLAG™, polyhistidine (His), myc, tandem affinity purification (TAP), or hemagglutinin (HA) tags or any detectable amino acid sequence. In embodiments, the component of the CRISPR system or CRISPR-Cas complex that is associated with a detectable agent is the pre-gRNA or mature gRNA.

In certain embodiments, the present invention provides methods for the treatment or prevention of C9orf72-mediated diseases in a subject, patient or individual in need thereof.

The terms ‘treating’ or ‘treatment’ as used herein refer to reducing the severity and/or frequency of symptoms, reducing the underlying pathological markers, eliminating symptoms and/or pathology, arresting the development or progression of symptoms and/or pathology, slowing the progression of symptoms and/or pathology, eliminating the symptoms and/or pathology, or improving or ameliorating pathology/damage already caused by the disease, condition or disorder.

The terms ‘preventing’ or ‘prophylaxis’ as used herein refer to the prevention of the occurrence of symptoms and/or pathology, delaying the onset of symptoms and/or pathology. Therefore, ‘preventing’ or ‘prophylaxis’ in particular, applies when a patient or subject has C9orf72 expansion repeats but does not yet display symptoms or pathology.

As used herein, ‘treatment or prevention of a C9orf72-mediated disease’ is referring to the use of the disclosed CRISPR system, complex, composition or vector to treat or prevent a disease, disorder or condition in a patient or subject with C9orf72 hexanucleotide expansion repeats.

As used herein, the terms ‘composition’ or ‘pharmaceutical composition’ are used interchangeably and refer to any composition comprising one or more guides, pre-gRNAs, mature gRNAs or pre-gRNA arrays in combination with a Cas effector protein, such as Cas13d or CasRx. In embodiments, the composition may further comprise a pharmaceutically acceptable carrier, diluent, adjuvants or excipient. As used herein the term ‘pharmaceutically acceptable carrier, diluent or excipient’ is intended to include sterile solvents or powders, dispersion media, coatings, antibacterial and antifungal agents, disintegrating agents, lubricants, glidant, sweeting or flavouring agents, antioxidants, buffers, chelating agents, binding agents, isotonic and absorption delaying agents, or suitable mixtures thereof. Preferably, the diluent or carrier is sterile water, saline solution, fixed oils, polyethylene glycols, glycerine, propylene glycol, bacteriostatic water, phosphate-buffered saline (PBS), Cermophor EL™ (BASF, Parsippany, N.J), other solvents or suitable mixtures thereof. Preferably, the antibacterial or antifungal agents include benzyl alcohol, parabens, chlorobutanol, phenol, ascorbic acid, thimerosal, methyl parabens, or suitable mixtures thereof. In embodiments, the antioxidants include ascorbic acid or sodium bisulphite of suitable mixtures thereof. In embodiments, the chelating agents include ethylenediaminetetraacetic acid (EDTA). In embodiments, the absorption delaying agents include aluminium monostearate or gelatin, or suitable mixtures thereof. In embodiments, isotonic agents include sugars, mannitol, sorbitol, or sodium chloride, or suitable mixtures thereof. In embodiments, the binding agent is microcrystalline cellulose, gum tragacanth, or gelatin, or suitable mixtures thereof. In embodiments, the excipient may be starch or lactose, or suitable mixtures thereof. In embodiments, the disintegrating agent may be alginic acid, Primogel or corn starch, or suitable mixtures thereof. In embodiments, the lubricant may be magnesium stearate or sterotes, or suitable mixtures thereof. In embodiments, the glidant may be colloidal silicon dioxide. In embodiments, the sweetening or flavouring agents may be sucrose, saccharin, peppermint, methyl salicylate or orange flavouring.

As used herein, the terms ‘administering’, ‘administer’ or ‘administration’ means providing to a subject or patient the complex, composition or vector using any method of delivery known to those skilled in the art to treat or prevent a disease, disorder or condition in a patient or subject with C9orf72 hexanucleotide expansion repeats. Preferred routes of delivery of the complex, composition or vector include intravenous, intradermal, subcutaneous, intraperitoneal, intramuscular, intrathecal or direct injection into the brain, inhalation, rectally (suppository or retention enema), vaginally, orally (capsules, tablets, solutions or troches), transmucosal or transdermal (topical e.g., skin patches, opthalamic, intranasal) application. In alternative embodiment, the complex, composition or vector is delivered directly to the cerebrospinal fluid (CSF), or brain, by a route of administration such as intrastriatal (IS), or intracerebroventricular (ICV) administration. The complex, composition or vector can also be administered by any method suitable for administration of nucleic acid agents, such as a DNA vaccine. These methods include gene guns, bio-injectors, and needle-free methods such as the mammalian transdermal needle-free vaccination with powder-form vaccine as disclosed in U.S. Pat. No. 6,168,587. If desired to facilitate repeated or frequent infusions, implantation of a delivery device, e.g., a pump, semi-permanent stent (e g., intravenous, intraperitoneal, intracisternal or intracapsular), or reservoir may be used. In embodiments encompassing inhalation, the complex, composition or vector are delivered in the form of an aerosol spray from a pressured container or dispenser which contains a suitable propellant or nebuliser. In embodiments the propellant may be a gas such as carbon dioxide.

As used herein, the term ‘therapeutically effective amount’ or ‘therapeutically effective dose’ refers to an amount of a complex, composition or vector that, when administered to a patient or subject with a C9orf72-mediated disease, is sufficient to cause a qualitative or quantitative reduction in the severity or frequency of symptoms of that disease, disorder or condition, and/or a reduction in the underlying pathological markers or mechanisms. In addition, a ‘therapeutically effective amount’ also refers to an amount of a complex, composition or vector that, when administered to a patient or subject with GGGGCC hexanucleotide repeat expansions without symptoms, is sufficient to cause a qualitative or quantitative reduction in the underlying pathology markers or mechanisms.

In a preferred embodiment, the therapeutically effective amount of complex, composition or vector may be administered only once. Preferably, the therapeutically effective amount of complex, composition or vector of the present invention is administered multiple times. In one embodiment, a patient or subject is administered an initial dose, and one or more maintenance doses. Certain factors may influence the dosage required to effectively treat a subject or patient, including but not limited to the severity of the disease, disorder or condition, previous or concurrent treatments, the general health and/or age of the subject, and other diseases present. It will also be appreciated that the effective dosage of the complex, composition or vector for treatment may increase or decrease over the course of a particular treatment.

In an alternative embodiment, the therapeutically effective dose may be administered with other therapies for ALS and FTD. Example secondary therapies can be to alleviate symptoms, neuroprotective, or restorative. Further methods and compositions (e.g. vectors and pharmaceutical preparations, and doses thereof) suitable generally for treating diseases using CRISPR/Cas-mediated delivery are described in or may be determined with reference to e.g. WO 2017/091630 and WO2019/084140, the contents of which are incorporated by reference. Such methods and compositions may be modified for use in the present invention where appropriate.

The invention will now be described by way of example only, with reference to the following non-limiting embodiments.

### EXAMPLES

### Example 1: Design of C9orf72 CasRx Guide RNAs

The majority of evidence suggests that C9orf72-related FTD/ALS is caused by a toxic gain of function (Mizielinska et al., 2014; Saberi et al., 2017; Stopford et al., 2017; Suzuki et al., 2018), however C9orf72 patients have a reduced expression of C9orf72 (˜50%) suggesting a potential loss of function contribution to disease pathogenesis (Jackson et al., 2020; Rizzu et al., 2016). Loss or reduction of C9orf72 function has been shown to exacerbate the gain of function mechanisms of the hexanucleotide expansion repeat with increased DPR accumulation, glial activation, and hippocampal neuron loss in a mouse model (Zhu et al., 2020). Therefore, to minimise any further reduction in C9orf72 expression, the guide RNA (gRNA) sequences were targeted to the sequence upstream of the hexanucleotide repeat expansion which should result in targeting only transcript variants 1 and 3, while leaving variant 2 intact (FIG. 1).

The gRNAs were designed taking into account predicted off-target scores and gRNA secondary structure. Off-target scores were determined using the Basic Local Alignment Search Tool (BLAST) against the human transcriptome, and RNA secondary structure scores were predicted using RNAfold Webserver (University of Vienna).

The sequences for the C9orf72 CasRx gRNAs are shown below. The sequences labelled ‘spacer’ indicate the targeting guide sequence alone (bold; see FIG. 4). Sequences labelled ‘Pre-gRNA+spacer’ indicate the immature pre-gRNA sequence as found in a plasmid or vector. The pre-gRNA sequence (underlined) is the same for all of the guides (see FIG. 4). When the pre-gRNA+spacer sequence is matured, around eight nucleotides are removed from the 3′ end of the spacer, and the direct repeat is truncated. The sequences labelled ‘gRNA+spacer’ indicate the expected mature gRNA found in the cell. Also indicated is the location of where on the C9orf72 gene sequence (SEQ ID NO: 56) each guide sequence targets (numbering is according to the presented nucleotide sequence for C9orf72).

Guide 1 (sense):
Custom spacer only:
(SEQ ID NO: 1)
CTTGTTCACCCTCAGCGAGTACTGTGAGAG
Pre-gRNA + spacer:
(SEQ ID NO: 2)
CAAGTAAACCCCTACCAACTGGTCGGGGTTTGAAACCTTGTTCACCCTCAGCGA
GTACTGTGAGAG
Mature gRNA + spacer:
(SEQ ID NO: 3)
AACCCCTACCAACTGGTCGGGGTTTGAAACCTTGTTCACCCTCAGCGAGTAC
Targets base pairs 201-230 on the C9orf72 gene sequence (SEQ ID NO: 56).
Guide 2 (sense):
Custom spacer only:
(SEQ ID NO: 4)
CAGGTCTTTTCTTGTTCACCCTCAGCGAGT
Pre-gRNA + spacer:
(SEQ ID NO: 5)
CAAGTAAACCCCTACCAACTGGTCGGGGTTTGAAACCAGGTCTTTTCTTGTTCA
CCCTCAGCGAGT
Mature gRNA + spacer:
(SEQ ID NO: 6)
AACCCCTACCAACTGGTCGGGGTTTGAAACCAGGTCTTTTCTTGTTCACCCT
Targets base pairs 211-240 on the C9orf72 gene sequence (SEQ ID NO: 56).
Guide 3 (Sense):
Custom spacer only:
(SEQ ID NO: 7)
TAATCTTTATCAGGTCTTTTCTTGTTCACC
Pre-gRNA + spacer:
(SEQ ID NO: 8)
CAAGTAAACCCCTACCAACTGGTCGGGGTTTGAAACTAATCTTTATCAGGTCTT
TTCTTGTTCACC
Mature gRNA + spacer:
(SEQ ID NO: 9)
AACCCCTACCAACTGGTCGGGGTTTGAAACTAATCTTTATCAGGTCTTTTCT
Targets base pairs 221-250 on the C9orf72 gene sequence (SEQ ID NO: 56).
Guide 4 (Sense):
Custom spacer only:
(SEQ ID NO: 10)
TTCTTCTGGTTAATCTTTATCAGGTCTTTT
Pre-gRNA + spacer:
(SEQ ID NO: 11)
CAAGTAAACCCCTACCAACTGGTCGGGGTTTGAAACTTCTTCTGGTTAATCTTT
ATCAGGTCTTTT
Mature gRNA + spacer:
(SEQ ID NO: 12)
AACCCCTACCAACTGGTCGGGGTTTGAAACTTCTTCTGGTTAATCTTTATCA
Targets base pairs 231-260 on the C9orf72 gene sequence (SEQ ID NO: 56).
Guide 5 (sense):
Custom spacer only:
(SEQ ID NO: 13)
CCTCCTTGTTTTCTTCTGGTTAATCTTTAT
Pre-gRNA + spacer:
(SEQ ID NO: 14)
CAAGTAAACCCCTACCAACTGGTCGGGGTTTGAAACCCTCCTTGTTTTCTTCTG
GTTAATCTTTAT
Mature gRNA + spacer:
(SEQ ID NO: 15)
AACCCCTACCAACTGGTCGGGGTTTGAAACCCTCCTTGTTTTCTTCTGGTTA
Targets base pairs 241-270 on the C9orf72 gene sequence (SEQ ID NO: 56).
Guide 6 (sense):
Custom spacer only:
(SEQ ID NO: 16)
CGGTTGTTTCCCTCCTTGTTTTCTTCTGGT
Pre-gRNA + spacer:
(SEQ ID NO: 17)
CAAGTAAACCCCTACCAACTGGTCGGGGTTTGAAACCGGTTGTTTCCCTCCTTG
TTTTCTTCTGGT
Mature gRNA + spacer:
(SEQ ID NO: 18)
AACCCCTACCAACTGGTCGGGGTTTGAAACCGGTTGTTTCCCTCCTTGTTTT
Targets base pairs 251-280 on the C9orf72 gene sequence (SEQ ID NO: 56).
Guide 7 (sense):
Custom spacer only:
(SEQ ID NO: 19)
CTACAGGCTGCGGTTGTTTCCCTCCTTGTT
Pre-gRNA + spacer:
(SEQ ID NO: 20)
CAAGTAAACCCCTACCAACTGGTCGGGGTTTGAAACCTACAGGCTGCGGTTGT
TTCCCTCCTTGTT
Mature gRNA + spacer:
(SEQ ID NO: 21)
AACCCCTACCAACTGGTCGGGGTTTGAAACCTACAGGCTGCGGTTGTTTCCC
Targets base pairs 261-290 on the C9orf72 gene sequence (SEQ ID NO: 56).
Guide 8 (Sense):
Custom spacer only:
(SEQ ID NO: 22)
CCAGAGCTTGCTACAGGCTGCGGTTGTTTC
Pre-gRNA + spacer:
(SEQ ID NO: 23)
CAAGTAAACCCCTACCAACTGGTCGGGGTTTGAAACCCAGAGCTTGCTACAGG
CTGCGGTTGTTTC
Mature gRNA + spacer:
(SEQ ID NO: 24)
AACCCCTACCAACTGGTCGGGGTTTGAAACCCAGAGCTTGCTACAGGCTGCG
Targets base pairs 271-300 on the C9orf72 gene sequence (SEQ ID NO: 56).
Guide 9 (sense):
Custom spacer only:
(SEQ ID NO: 25)
CTCCTGAGTTCCAGAGCTTGCTACAGGCTG
Pre-gRNA + spacer:
(SEQ ID NO: 26)
CAAGTAAACCCCTACCAACTGGTCGGGGTTTGAAACCTCCTGAGTTCCAGAGC
TTGCTACAGGCTG
Mature gRNA + spacer:
(SEQ ID NO: 27)
AACCCCTACCAACTGGTCGGGGTTTGAAACCTCCTGAGTTCCAGAGCTTGCT
Targets base pairs 281-310 on the C9orf72 gene sequence (SEQ ID NO: 56).
Guide 10 (sense):
Custom spacer only:
(SEQ ID NO: 28)
TAGCGCGCGACTCCTGAGTTCCAGAGCTTG
Pre-gRNA + spacer:
(SEQ ID NO: 29)
CAAGTAAACCCCTACCAACTGGTCGGGGTTTGAAACTAGCGCGCGACTCCTGA
GTTCCAGAGCTTG
Mature gRNA + spacer:
(SEQ ID NO: 30)
AACCCCTACCAACTGGTCGGGGTTTGAAACTAGCGCGCGACTCCTGAGTTCC
Targets base pairs 291-320 on the C9orf72 gene sequence (SEQ ID NO: 56).
Guide 11 (antisense):
Custom spacer only:
(SEQ ID NO: 31)
CGCAGGCGGTGGCGAGTGGGTGAGTGAGGA
Pre-gRNA + spacer:
(SEQ ID NO: 32)
CAAGTAAACCCCTACCAACTGGTCGGGGTTTGAAACCGCAGGCGGTGGCGAGT
GGGTGAGTGAGGA
Mature gRNA + spacer:
(SEQ ID NO: 33)
AACCCCTACCAACTGGTCGGGGTTTGAAACCGCAGGCGGTGGCGAGTGGGTG
Targets base pairs 418-447 on the C9orf72 gene sequence (SEQ ID NO: 56).
Guide 12 (antisense):
Custom spacer only:
(SEQ ID NO: 34)
TGCGCCCGCGGCGGCGGAGGCGCAGGCGGT
Pre-gRNA + spacer:
(SEQ ID NO: 35)
CAAGTAAACCCCTACCAACTGGTCGGGGTTTGAAACTGCGCCCGCGGCGGCGG
AGGCGCAGGCGGT
Mature gRNA + spacer:
(SEQ ID NO: 36)
AACCCCTACCAACTGGTCGGGGTTTGAAACTGCGCCCGCGGCGGCGGAGGCG
Targets base pairs 398-427 on the C9orf72 gene sequence (SEQ ID NO: 56).
Guide 13 (antisense):
Custom spacer only:
(SEQ ID NO: 37)
TTAACTTTCCCTCTCATTTCTCTGACCGAA
Pre-gRNA + spacer:
(SEQ ID NO: 38)
CAAGTAAACCCCTACCAACTGGTCGGGGTTTGAAACTTAACTTTCCCTCTCATT
TCTCTGACCGAA
Mature gRNA + spacer:
(SEQ ID NO: 39)
AACCCCTACCAACTGGTCGGGGTTTGAAACTTAACTTTCCCTCTCATTTCTC
Targets base pairs 539-567 on the C9orf72 gene sequence (SEQ ID NO: 56).
Guide 14 (antisense):
Custom spacer only:
(SEQ ID NO: 40)
TTCGGCTGCCGGGAAGAGGCGCGGGTAGAA
Pre-gRNA + spacer:
(SEQ ID NO: 41)
CAAGTAAACCCCTACCAACTGGTCGGGGTTTGAAACTTCGGCTGCCGGGAAGA
GGCGCGGGTAGAA
Mature gRNA + spacer:
(SEQ ID NO: 42)
AACCCCTACCAACTGGTCGGGGTTTGAAACTTCGGCTGCCGGGAAGAGGCGC
Targets base pairs 478-507 on the C9orf72 gene sequence (SEQ ID NO: 56).
Guide 17 (antisense):
Custom spacer only:
(SEQ ID NO: 43)
TCCCTCTCATTTCTCTGACCGAAGCTGGGT
Pre-gRNA + spacer:
(SEQ ID NO: 44)
CAAGTAAACCCCTACCAACTGGTCGGGGTTTGAAACTCCCTCTCATTTCTCTGA
CCGAAGCTGGGT
Mature gRNA + spacer:
(SEQ ID NO: 45)
AACCCCTACCAACTGGTCGGGGTTTGAAACTCCCTCTCATTTCTCTGACCGAA
Targets base pairs 545-574 on the C9orf72 gene sequence (SEQ ID NO: 56).
Sequences common to all guides (C9orf72-targeting guides)
Pre-gRNA direct repeat sequence:
(SEQ ID NO: 46)
CAAGTAAACCCCTACCAACTGGTCGGGGTTTGAAAC
Mature gRNA direct repeat sequence:
(SEQ ID NO: 47)
AACCCCTACCAACTGGTCGGGGTTTGAAAC
Non-targeting guide:
(SEQ ID NO: 77)
Spacer: GTAATGCCTGGCTTGTCGACGCATAGTCTG

The non-targeting control guide sequence (SEQ ID NO: 77) has no homology to the human transcriptome and has been published previously (Cox et al., 2018. RNA editing with CRISPR-Cas13. Science, 358(6366): 1019-1027. DOI: 10.1126/science.aaq0180). Using a non-targeting guide sequence as a control confirms that any effect observed is due to the targeting of the C9orf72 transcripts and not due to the over expression of CasRx.

### Annealing

The spacer guide sequences were ordered as oligonucleotides from Sigma. The oligonucleotides were annealed in annealing buffer (1 mM ethylenediaminetetraacetic acid (EDTA), 50 mM NaCl, 10 mM Tris pH 7.5) by heating for 2 minutes to 95° C., then cooled stepwise to room temperature for 3 hours. Once annealed, the guides have overhangs to facilitate restriction enzyme cloning into their respective expression plasmids.

### Example 2: Plasmid Design

The sequences of all starting plasmids were confirmed via Sanger sequencing (Source Bioscience, UK) using the primers in Table 1 below.

TABLE 1
Primers for PCR and Sanger Sequencing.

| | SEQ | |
|---|---|---|
| Primer Name | ID NO. | Sequence (5′-3′) |
| U6 gRNA PacI PCR | 48 | GCATTAATTAATTACGGTTCCTGGCCTTTTG |
| Cloning Forward | | |
| U6 gRNA PacI PCR | 49 | GCATTAATTAACGTAAGGAGAAAATACCGCATCA |
| Cloning Reverse | | |
| AS55RNL Seq | 50 | TTCCTAGCAACCCCGACTTG |
| Forward | | |
| AS55RNL Seq | 51 | TTGGATCGGAGTTACGGACACC |
| Reverse | | |
| Ef1a Seq Forward | 52 | TCAAGCCTCAGACAGTGGTTC |
| U6 Seq Forward | 53 | GACTATCATATGCTTACCGT |

As pure G₄C₂ repeat expansions are not possible to sequence due to the high GC content, the G₄C₂ repeat lengths were estimated by analysing DNA gel band sizes following restriction digestion. All restriction digestions were performed according to manufacturer's instructions for each restriction enzyme.

### General Method for Plasmid Preparation

The backbone plasmid fragments and inserts were digested with restriction digestion (details in the detailed descriptions below). Plasmid backbone fragments were then dephosphorylated using calf intestinal phosphatase (CIP; NEB, M0290) to prevent re-ligation. Insert fragments were left with 5′ phosphate groups intact to aid ligation. The digested backbone plasmid and insert were then run on a 0.8%-1% agarose gel at 110 volts for ˜1 hour and desired fragments were excised from the gel and the plasmid backbone DNA extracted from the excised gel using DNA gel extraction kit (Qiagen, 28115).

The desired inserts and plasmid backbones were then ligated using the T4 DNA ligase (NEB, M0202) according to manufacturer's instructions with various molar ligation ratios which had been optimised (usually 3:1-9:1 insert: backbone).

Ligated fragments were then transformed into chemically competent E. coli cells, according to the manufacturer instructions. One Shot™ TOP10 E. coli (ThermoFisher Scientific, C404003) were used for stable plasmids (such as the gRNA expression plasmids), and One Shot™ Stbl3™ E. coli (ThermoFisher Scientific, C737303) were used for unstable plasmids (such as repeat containing plasmids and lentiviral plasmids). Transformed E. coli were then plated on Luria-Bertani (LB) agar (Sigma, L2025) plates containing 100 μg/mL of ampicillin (Sigma, A9518) for selection. Colonies were picked after 24 hours of growth and grown in 5 mL Luria Broth (LB; Sigma, L3522) for stable plasmids, or low salt LB (Sigma, L3397) for unstable plasmids, at 37° C. at 225 rpm overnight. Mini-preps (Qiagen, 27106) were performed on a sample of the bacteria the following day following the manufacturer instructions. Restriction digestions and gel electrophoresis were then performed to check the band sizes to determine which bacterial samples comprised the correctly ligated DNA fragments. Samples with the correct bands were confirmed via Sanger sequencing using the primers outlined in Table 1. Bacteria comprising the correct plasmids were Maxi-prepped (Qiagen, 12362) with an endotoxin removal buffer according to the manufacturer instructions and the plasmids stored at −20° C. until required.

### Producing the Sense and Antisense Nanoluciferase Reporter Constructs

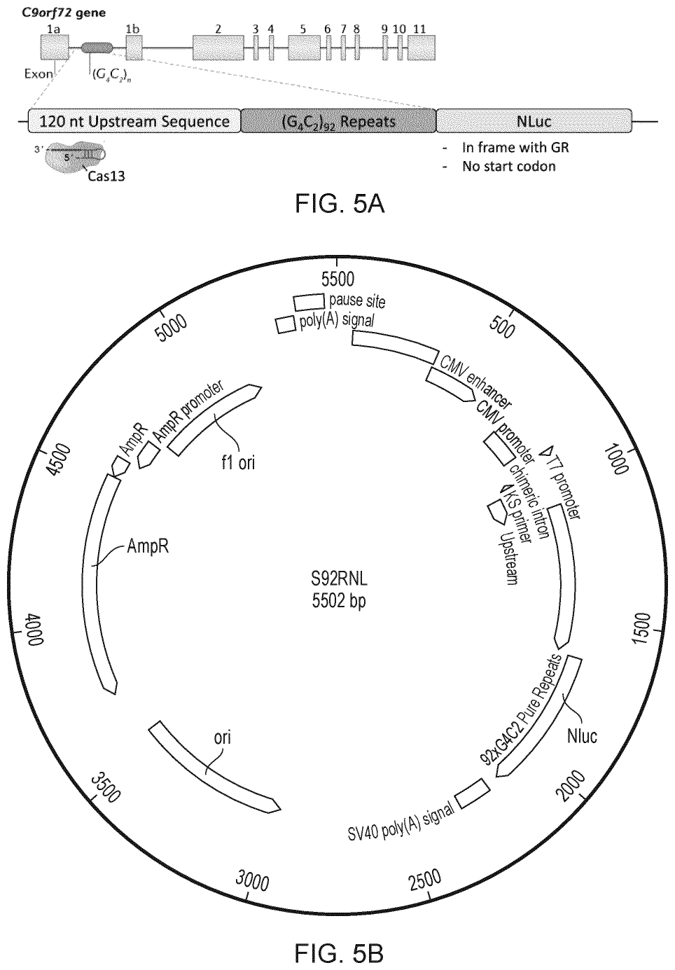

A sense repeat-associated non-AUG (RAN) translation Nanoluciferase (NLuc) reporter construct referred to as S92RNL (Sense GR-Nanoluciferase reporter plasmid; SEQ ID NO: 57) contains 92 pure G₄C₂ repeats with 120 nucleotides of the endogenous upstream C9orf72 sequence and a NLuc in frame with poly-GR (FIG. 5A and 5B).

To generate the antisense RAN translation NLuc reporter construct, an insert sequence was designed and ordered from GeneArt (ThermoFisher Scientific) which contained 680 nucleotides (nt) 5′ of the endogenous antisense C9orf72 repeat sequence (corresponding to base pairs 343 to 1022 of SEQ ID NO: 56) along with the restriction sites for EcoRV (NEB, R0195) and SpeI (NEB, R0133) to facilitate cloning into the NLuc backbone expression plasmid (Isaacs Lab). 680 base pairs of the endogenous C9orf72 upstream sequence 5′ of the hexanucleotide repeats were included in the AS55NL plasmid (SEQ ID NO: 58) as the transcription start site for the antisense transcript is unknown, however it has been suggested to be as far as 600 base pairs 5′ of the repeats (Rizzu et al., 2016). Restriction sites for NotI (NEB, R3189) and BspQI (NEB, R0712) were included in this insert sequence to allow the cloning of the G₄C₂ repeats from the S92RNL plasmid in the reverse order to produce C₄G₂ repeats with the endogenous upstream sequence.

The NLuc backbone expression plasmid was linearised and the band at size 5.4 kb was excised. The insert sequence was then ligated into the NLuc backbone expression plasmid as described above with a 3:1 ligation ratio.

The NLuc backbone expression plasmid utilises a unidirectional origin of replication (ORI), which resulted in the G-rich region of the repeats being in the lagging strand when the repeats were flipped during cloning to produce antisense repeats. G-rich regions present in the lagging strand are commonly truncated during replication, therefore increasing the chances of reducing the repeat length. Our attempts to reverse the ORI to prevent the shortening of the repeats were unsuccessful. Despite this we managed to retain ˜55 repeats, confirmed by DNA band size of 580 bp on an agarose gel after digestion with SpeI and EcoRV.

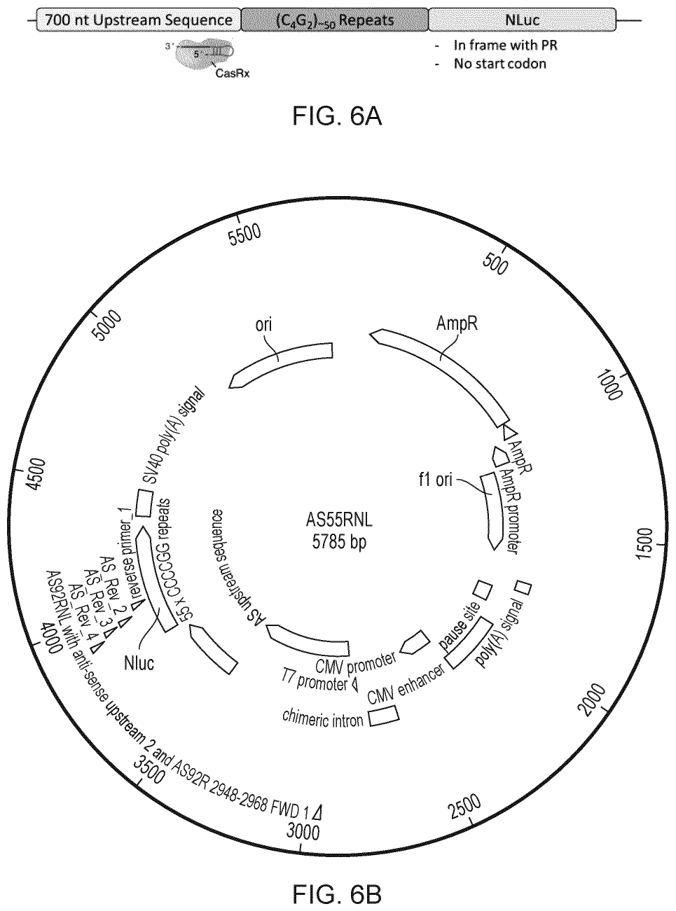

To minimise the risk of the repeat length reducing, Stbl3 E.coli were used for transformations and were grown in low salt LB at room temperature without shaking following the protocol outlined above. Correctly ligated plasmids were obtained following the protocol outlined above. The resulting antisense plasmid is referred to as AS55RNL (Antisense PR-Nanoluciferase reporter plasmid with ˜55 C4G2 repeats) and is shown in FIG. 6.

### Cloning of gRNAs into a gRNA Expression Plasmid

Annealed spacer oligonucleotides from Example 1 (SEQ ID NOs: 1, 4, 7, 10, 13, 16, 19, 22, 25, 28, 31, 34, 37, 40 or 43) were designed to have the correct overhangs for cloning into the pXR003 gRNA expression backbone vectors.

The backbone vector was linearised through restriction digestion with BbsI-HF (NEB, R0539) as outlined above and identified through gel electrophoresis. The backbone vector and the annealed spacer sequences were then ligated following the protocol outlined above. Correct ligation was determined via restriction digestion with BbsI. As BbsI is a Type IIs restriction enzyme, if guides are successfully ligated, then the BbsI restriction site will be removed. Agarose gel electrophoresis was then used to identify the complete plasmid as the ligated plasmid will not linearise.

### gRNA/CasRx Lenti-Viral Plasmid

In order to express the CasRx and the gRNA from the same lentiviral plasmid, the U6 promoter, direct repeats, and gRNA sequences were PCR'd out from the complete guide expressing vectors detailed above, leaving PacI restriction site overhangs to allow for cloning into the CasRx expressing lentiviral vector (pXR001). To achieve this, primers with SEQ ID NOs: 48 and 49 were used, and PCR amplification performed using a modified Pfu DNA polymerase as shown in Table 2 below (PCRBIO VeriFi™ Mix; PCR Biosystems, PB10.43-01).

TABLE 2
PCR reaction Conditions.

| Temperature (° C.) | Time | Cycles |
|---|---|---|
| 95 | 3 mins | 1 |
| 95 | 15 secs | 10 |
| 65 | 15 secs | |
| 72 | 15 secs | |
| 95 | 15 secs | 25 |
| 69 | 15 secs | |
| 72 | 15 secs | |
| 72 | 5 mins | 1 |

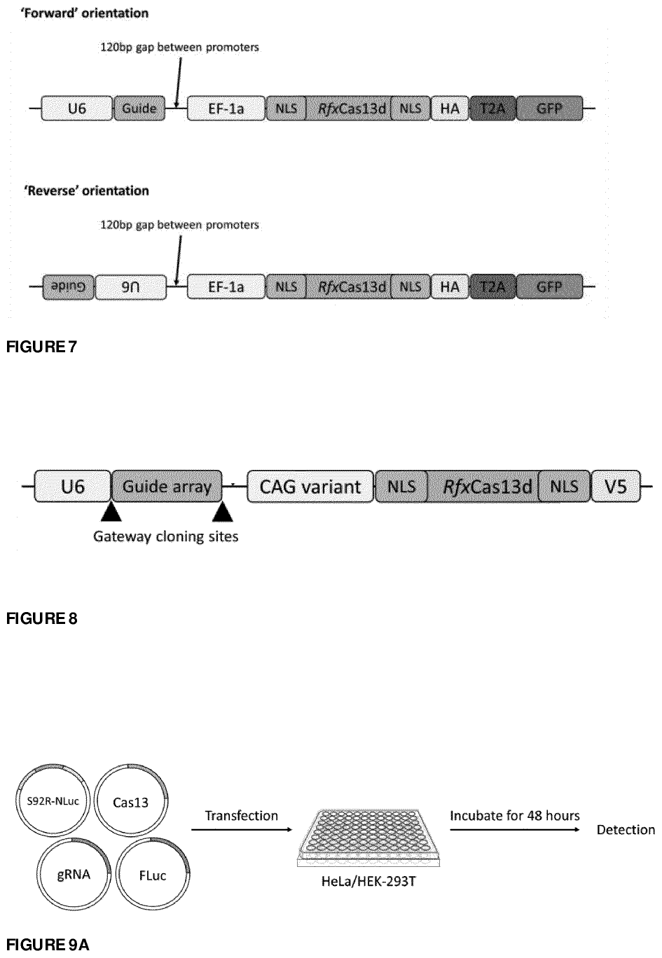

The resulting PCR product was then purified using a PCR purification kit (Qiagen, 28104) and ligated into the pJET1.2 cloning vector using the CloneJet PCR cloning kit (ThermoFisher Scientific, K1231) following the manufacturer's instructions. The resulting vector was then transformed into One Shot™ Stbl3™ E. coli and grown as outlined above. Plasmid DNA were isolated via mini-prep as outlined above, and the correct 395 bp fragment was digested out of pJET1.2 cloning vector with PacI-HF (NEB, R0547) and electrophoresis performed in a 0.8% agarose gel at 100V for 1.5 hours. The CasRx expressing lentiviral vector (pXR0001) was digested with PacI. Due to there being only one PacI restriction site in the CasRx backbone, the U6 gRNA insert will ligate in both orientations (see FIG. 7 for diagrammatic representation).

### AAV9 Plasmid

An insert sequence containing the pre-gRNA array (multiple pre gRNAs+spacer sequences of either two non-targeting guide RNAs (i.e., two sequences of SEQ ID NO: 77) or guides 10 (SEQ ID NO: 29) and 17 (SEQ ID NO: 44)) and CasRx (ordered from GeneArt (Thermo Fisher)) are cloned into an AAV backbone vector using restriction sites NotI and AscI. Once inserted into the AAV backbone vector, Golden Gate cloning (Engler C, Kandzia R, Marillonnet S. (2008). A one pot, one step, precision cloning method with high throughput capability. PLOS One; 3(11): e3647. doi: 10.1371/journal.pone.0003647. Epub.) utilising type IIs restriction enzymes (BsmBI in this case) is used to clone in the guide array of choice without leaving any unwanted sequence from the cloning site (see FIG. 8 for diagrammatic representation of AAV design).

### Example 3: Cell Culture, Transfection and Detection Methods

### Immortalised Cell Culture

Human embryonic kidney 293 T (HEK293T; UCL Drug Discovery Institute), HeLa (cervical cancer cells from Henrietta Lacks), and HeLa A1 (HeLa cells that have been clonally selected for having higher RAN translation levels) cell lines were maintained in Dulbecco's modified eagle media (DMEM; ThermoFisher Scientific, 11960044) supplemented with 10% fetal bovine serum (FBS; ThermoFisher Scientific, A4766), 4.5 g/L glucose, 110 mg/L sodium pyruvate (ThermoFisher Scientific, 11360070), 1× GlutaMAX™ (ThermoFisher Scientific, 35050061) and kept at 37° C. with 5% CO2 to ensure physiological temperature and pH. Phenol red in the media was used to monitor pH. Cells were maintained up to a confluency of 90% and then dissociated and passaged with 0.05% Trypsin-EDTA. All cell lines were routinely tested for mycoplasma contamination with MycoAlert assay (Lonza).

### iPSC Donors & Reprogramming

Biopsy tissue was gathered with prior informed consent from patients. Ethical approval for the gathering of tissue for research purposes was received from the National Hospital for Neurology and Neurosurgery and the Institutional Review Board of the University of Edinburgh, and approval for use of patient-derived induced pluripotent stem cells (iPSCs) was received from UCL Institute of Neurology Joint Research Ethics Committee (09/H0716/64).

Patient-derived iPSCs were generated by either the laboratory of Professor Wray (UCL) or Professor Chandran (The University of Edinburgh) (Table 3) via reprogramming of patient fibroblasts as described elsewhere (Okita et al. (2011). A more efficient method to generate integration-free human iPS cells. Nature Methods, 8(5), 409-412. https://doi.org/10.1038/nmeth.1591). In brief, fibroblasts were retrovirally transduced or transfected with episomal plasmids to express Oct3/4, Sox2, Klf4, and c-Myc or L-Myc with suppression of p53 to induce pluripotency. Newly generated lines were tested for karyotypic abnormalities (The Doctor's Laboratory, London).

TABLE 3
iPSC donor information

| iPSC | | | Age of | Age of | No. of G₄C₂ | |
|---|---|---|---|---|---|---|
| Name | Gender | Diagnosis | onset (yrs) | death (yrs) | repeats | Source |
| BS6 | F | ALS/FTD | NA | NA | ~750 | Prof. Chandran |
| BS6 H9 | Isogenic control of BS6 | | | | ~2 | Prof. Chandran |
| DN19 | M | ALS | 52 | 58 | ~640 | Prof. Wray |
| DN19 | Isogenic control of DN19 | | | | ~2 | Prof. Chandran |
| V4D9 | | | | | | |
| M211R2 | M | ALS | NA | NA | ~960 | Prof. Chandran |
| M211R2 | Isogenic control of M211R2 | | | | 1 | |
| D9 | | | | | | |

### iPSC Culture and Differentiation

All iPSC lines were routinely tested for mycoplasma contamination with MycoAlert assay (Lonza, LT07-218). iPSCs were maintained in Essential 8 medium (E8; ThermoFisher Scientific, A1517001) supplemented with 1:50 E8 supplement (ThermoFisher Scientific, A1517001) on Geltrex-coated (ThermoFisher Scientific, A1413201) plates at 37° C. with 5% CO2. iPSCs were passaged at ˜80% confluency via a phosphate-buffered saline (PBS) wash followed by chelation of cations with EDTA for ˜5 minutes to lift cells from the plate. EDTA was aspirated and fresh E8 medium was applied and cells were transferred to new wells.

iPSCs were induced to form neuronal progenitor cells (NPCs) according to a protocol to produce motor neurons published previously (Hall et al. (2017). Progressive Motor Neuron Pathology and the Role of Astrocytes in a Human Stem Cell Model of VCP-Related ALS. Cell Reports, 19(9), 1739-1749. https://doi.org/10.1016/j.celrep.2017.05.024). In brief, iPSCs were induced with N2B27 media (Table 4) supplemented with SB431541 (2 μM), CHIR99021 (3.3 μM), and dorsomorphin (1 μM); referred to as induction media. Five days post-induction, cells were split 1:2 using a 15-minute treatment of Dispase II, whilst being careful not to dissociate the cells. The cells were transferred to a falcon tube containing PBS and DNase (2000 Units; ThermoFisher Scientific, EN0521) and washed a further two times with PBS, each time allowing the cells to settle to the bottom of the falcon tube. Cells were plated on Gel-trex-coated plates in induction media supplemented with 10 μM ROCK inhibitor (Y-27632; Selleckchem, S1049). Cell media was changed 7 days post-induction to patterning media, consisting of N2B27 medium supplemented with 0.5 μM retinoic acid (Sigma, R2625) and 1 μM purmorphamine (Sigma, SML0868). Cells were split again on day 12 following the protocol described above and cultured in patterning medium supplemented with ROCK inhibitor (10 μM). On day 18 post-induction, media was changed for N2B27 medium supplemented with 10 ng/ml human fibroblast growth factor (FGF; ThermoFisher Scientific, PHG0024), and cells were maintained in this medium as NPCs and used for experiments at this stage prior to terminal differentiation.

TABLE 4
N2B27 media composition.

| | Catalogue | Final concentration/ | |
|---|---|---|---|
| Product | Number* | amount | |
| DMEM/F12 medium | 10565018 | 1:2 | |
| Neurobasal medium | 21103049 | 1:2 | |
| Non-essential amino acids | 11140050 | 1:200 | |
| N2 supplement | 17502048 | 1:200 | |
| B27 supplement | 17504044 | 1:200 | |
| GlutaMAX ™ | 35050061 | 1 | mM |
| Insulin | A11382II | 1 | μg/ml |
| β-Mercaptoethanol | 31350010 | 50 | μM |

*All reagents sourced from ThermoFisher Scientific

NPC's from each induction were characterised via immunocytochemistry for Pax2, a transcription factor that indicates progenitor cells of a motor neuron lineage (Blake & Ziman.

(2014). Pax genes: Regulators of lineage specification and progenitor cell maintenance. Development (Cambridge), 141(4), 737-751. https://doi.org/10.1242/dev.091785).

### General Method for Transient Transfection of Immortalised Cells and NPCs

Immortalised cells and patient-derived NPCs were plated 24 hours prior to transfection. Immortalised cells were transiently transfected using 0.5 μL of Lipofectamine™ 2000 (ThermoFisher Scientific, 11668027) per well of a 96-well plate and in accordance with the manufacturer's instructions. Patient-derived NPCs were transfected using Lipofectamine™ Stem (ThermoFisher Scientific, STEM00008) according to manufacturer's instructions. Immortalised cells and NPCs were then incubated post-transfection at 37° C. with 5% CO2.

### Lentivirus Production

Low passage HEK293T cells were cultured as described above and plated in T175 flasks (ThermoFisher Scientific, 159910) at 50% confluency 24 hours prior to transfection. Cells were then transfected with 14.1 μg of PAX2 lentiviral packaging vector (Addgene, 12259), 9.36 μg of VSV.G lentiviral enveloping vector (Addgene, 8454) and 14.1 μg of the lentivirus plasmid comprising CasRx and pre-gRNA+spacer sequences as described in Example 2 using Lipofectamine™ 3000 (ThermoFisher Scientific, L3000008) according to the manufacturer instructions. Transfected cells were incubated post-transfection at 37° C. with 5% CO2.

48 hours after transfection, cell media was collected and stored at 4° C. and replaced with 30 mL of fresh media. After 24 hours the media was collected again. Both the stored and collected media were then centrifuged at 1500×g for 10 minutes at 4° C. and the supernatant collected. Lenti-X concentrator (Takara Bio, 631231) was added at 3:1 media to concentrator ratio and incubated for 72 hours at 4° C. The Lenti-X media mix was then centrifuged at 7000×g for 30 minutes at 4° C. and the supernatant discarded. The lentivirus pellet was then resuspended in 400 μL OptiMEM (ThermoFisher Scientific, 31985062) and aliquoted and stored at −80° C. until needed.

### Lentiviral Transduction

NPCs were plated in 12-well plates at a density of 500,000 cells per well 24 hours prior to transduction with 20 μL of concentrated lentivirus. Lentiviruses were removed via full media change 24 hours post-transduction. Cells were grown for a further 48 hours prior to lysis for downstream analysis.

### Firefly Luciferase and Nanoluciferase Reporter Assays

For dual-luciferase assays (Promega, N1630), HEK293T, or HeLa cells or patient-derived NPCs were plated at a density of 30,000 cells per well in a 96 well plate (for luciferase assays: Greiner Bio-One, 655083). The HEK293T or HeLa cells were then transiently transfected with 100 ng of the Cas13 gRNA plasmids (as described in Example 2), 25 ng of CasRx or Cas13b plasmids (Addgene, CasRx: 109049, Cas13b: 103862), 12.5 ng of Firefly luciferase expression plasmid (Promega, E5011), and 2.5 ng of RAN translation sense, antisense or control Nanoluciferase reporter plasmids (referred to as S92RNL (SEQ ID NO: 57), AS55RNL (SEQ ID NO: 58), or S0RNL (SEQ ID NO: 59) respectively) using 0.5 μL of Lipofectamine™ 2000 per well of a 96-well plate in accordance with the manufacturer's instructions. Transfection reagents were added directly to the media (10 μL per well of a 96 well plate) and left on for the duration of the experiment. The cells were then incubated post-transfection at 37° C. with 5% CO2. Each experiment consists of 3-5 technical replicate wells per condition.

48 hours post-transfection both Firefly and Nanoluciferase signals were measured using the Nano-Glo Dual Luciferase Assay according to manufacturer's instructions, on the FLUOstar Omega (BMG Labtech) with a threshold of 80% and a gain of 2000 for both readings. The Nanoluciferase reading was normalised to the Firefly luciferase reading for each well to control for variable transfection efficiencies.

### Combined Single Molecule RNA FISH and Immunocytochemistry

For RNA fluorescent in situ hybridisation (RNA FISH) in HEK293T cells and patient-derived NPCs, the cells were plated at a density of 25,000 cells per well in a 96 well plate and transfected with 100 ng of Cas13 gRNA plasmids (as described in Example 2), 25 ng of CasRx or Cas13b plasmids, 12.5 ng of Firefly luciferase expression plasmid (Promega, E5011) and 2.5 ng of RAN translation sense (S92RNL) or antisense (AS55RNL) Nanoluciferase reporter plasmids using 0.5 μL of Lipofectamine™ 2000 per well of a 96-well plate and in accordance with the manufacturer's instructions. Transfection reagents were added directly to the media, and each plate contained 3-5 technical replicates per condition. Cells were then incubated post-transfection at 37° C. with 5% CO2.

Cells were fixed 48 hours post-transfection for 7 minutes using 4% Paraformaldehyde (PFA; Sigma, F8775) with 10% methanol diluted in PBS with cations (ThermoFisher Scientific, A1285801) to aid cell adhesion. Cells were then dehydrated with 70% ethanol followed by 100% ethanol washes and frozen at −80° C. in 100% ethanol until needed. To perform the experiments, frozen cells were rehydrated with 70% ethanol and washed for 5 minutes at room temperature in pre-hybridisation solution (40% formamide (VWR, 97062-010), 2× saline sodium citrate (SSC; ThermoFisher Scientific, 15557044), 10% dextran sulphate (Sigma, D6001), 2 mM vanadyl ribonucleoside complex (Sigma, 94740). Cells were then permeabilised with 0.2% Triton X-100 (Sigma, X100) for 10 minutes. Cells were incubated at 60° C. in pre-hybridisation solution for 45 minutes. Locked nucleic acid (LNA) probes to detect either sense or antisense RNA-foci (Table 5) were then added to the pre-hybridisation solution at 40 nM and cells were kept in the dark at 60° C. or 66° C. (for sense and antisense probes, respectively) for 3 hours. Cells were then washed with 0.2% Triton-X100 in 2× SSC for 5 minutes at room temperature followed by 30 minutes at 60° C. One additional wash in 0.2× SSC at 60° C. for 30 minutes was performed prior to application of 647-conjugated HA antibody (in 0.2× SSC with 1% BSA (Sigma, A3311) at 1:1000; BioLegend, 682404) for detection of HA-tagged CasRx. This was incubated overnight at 4° C. and then washed with 0.2× SSC for 20 minutes at room temperature. Hoescht 34580 (ThermoFisher Scientific, H21486) was added at 1:5000 in 0.2× SSC for 10 minutes to detect cell nuclei. Hoescht solution was removed and cells were left in 0.2× SSC at 4° C. protected from light until imaging.

TABLE 5
RNA-FISH LNA Probes

| Probe | SEQ ID NO. | Sequence |
|---|---|---|
| Sense | 54 | CCCCGGCCCCGGCCCC |
| Antisense | 55 | GGGGCCGGGGCCGGGG |

Both probes were 5′TYE563-labelled

### Immunocytochemistry

Immortalised cells or patient-derived NPCs were cultured on clear bottomed 96 well plates (Cell Carrier Ultra, Perkin Elmer, 6055300) suitable for imaging on the Opera Phenix® (Perkin Elmer). Cells were transfected as previously described for RNA FISH experiments and left for 48 hours prior to fixing with the 4% PFA for 7 minutes. PFA was removed and cells were blocked and permeabilised at the same time with 10% FBS and 0.25% Triton X-100 in PBS (with cations) for 1 hour at room temperature. Cells were incubated with primary anti-HA antibody (Santa Cruz, sc-805) at 1:1000 in 10% FBS overnight at 4° C. Cells were washed three times the following day with PBS. A fluorophore conjugated secondary antibody (Alexa Flour 546; ThermoFisher Scientific, A11035) was added at 1:1000 in 10% FBS and incubated at room temperature for 1.5 hours protected from light. Cells were then washed three times with PBS prior to 10-minute incubation with 1 μg/ml Hoescht. Hoescht was removed and cells were stored in PBS at 4° C. and protected from light until imaging.

### Imaging Techniques and Quantification

RNA-FISH and immunocytochemistry experiments were all imaged using the automated Opera Phenix® high-throughput confocal imaging platform. Dual RNA-FISH and immunocytochemistry images were analysed using Columbus 2.8 (PerkinElmer) using a custom algorithm workflow to determine total RNA-foci load per cell. RNA-foci load was determined by calculating the integrated intensity of nuclear RNA puncta by multiplying spot intensity by total spot load per CasRx positive cell (as determined by nuclear HA positivity). Transfection efficiency images were taken using the IncuCyte live cell imager (Essen BioScience).

### Meso Scale Discovery Immunoassay (MSD)

In another example, the meso scale discovery immunoassay (MSD) is used to detect DPR proteins in patient-derived NPCs.

Protein is extracted from NPC samples using lysis buffer (1× radioimmunoprecipitation assay (RIPA) buffer (Sigma, R0278) with 2% sodium dodecyl sulfate (SDS; Sigma, 71725) and 2× protease inhibitor cocktail (Sigma, 1183617001)) and transferred to Eppendorf tubes followed by three sonications at 5 amps for five seconds each. Samples are then centrifuged at 20,000×g for 10 minutes at 4° C. and supernatant collected. Protein concentration is determined via BCA assay (ThermoFisher Scientific, A53225).

30 μL of capture antibody (Eurogentec, ZGB16103-Rb.658) in TBS (0.2% Tween® 20 (Sigma, P7949) with 3% milk powder in PBS) is added to each well of a multi-array 96 well plate (MSD, MSD L15XA-3/L11XA-3) and left to shake at 1250 rpm for 15 seconds followed by 600 rpm for 15 minutes. The plates are then incubated at 4° C. overnight. The following day the wells are washed three times with 150 μL tris-buffered saline (TBS) and blocked with TBS for 2 hours at room temperature at 600 rpm. Following the washes, 25 μL of Electrically Competent (EC) buffer (Isaacs Lab) is added with 0.3 μg of the NPC protein sample or a 7.5× GP repeats standard (Custom order from Eurogentec) and left to incubate overnight at 4° C. at 600 rpm. The following day, cells are washed three times with TBS followed by application of 25 μL per well of the detection antibody (Eurogentec, ZGB16103-Rb.658) in TBS. This is then incubated at room temperature for 2 hours at 600 rpm. Cells are washed as before prior to the application of 25 μL streptavidin-SULFO-tag (MSD, MSD R32AD-1), which is then incubated at room temperature for 1 hour at 600 rpm. To determine the target protein concentration, 150 μL of Reading Buffer T (MSD, MSD R92TC-1) is added per well to activate the fluorescent signal which is read using the MSD Sector Imager.

### Statistical Analyses and Data Presentation

Biologically independent experiment replicates (N number) are indicated in figure legends. Within each biological repeat, there was minimum of 3 technical replicates. Technical replicates are displayed on graphs as well biological replicates. Statistical analyses were only performed on experiments with 3 biologically independent replicates. All datasets conformed to Gaussian distribution as determined by Shapiro-Wilk normality testing, therefore parametric one-way or two-way ANOVA tests were carried out with Holm-Sidak post-hoc analysis (p=0.05) to determine statistical significance between test groups. Student's T-test was used to determine significance between two test groups (p=0.05). All statistical tests were carried out using GraphPad Prism 8.4.1. All data in text and figures, unless otherwise stated, are given as fold-change compared to non-targeting guide control. Data displayed as mean values±standard deviation (S.D.).

### Example 4: Mouse Models & Methods

### C9orf72 Mouse Model

The C9orf72 bacterial artificial chromosome (BAC) mouse model is as previously described in Liu et al. (2016. C9orf72 BAC Mouse Model with Motor Deficits and Neurodegenerative Features of ALS/FTD. Neuron, 90(3), 521-534. https://doi.org/10.1016/j.neuron.2016.04.005). These mice are available from The Jackson Laboratory (USA, #FVB/NJ-Tg (C9orf72) 500 Lpwr/J). These mice exhibit decreased survival, paralysis, muscle denervation, motor neuron loss, anxiety-like behaviour, and cortical and hippocampal neurodegeneration, along with RAN protein accumulation, and TDP-43 inclusions (Liu et al., 2016).

### Treatment of C9orf72 Mouse Model

C9orf72 mice are treated at 12-14 months with a PhP.eB AAV9 vector containing the CasRx therapy; consisting of the pre-gRNA and CasRx, as outlined in Example 2.

### Mouse Brain Protein Isolation

100 mg of frozen brain was taken per mouse and lysed with 0.9× tissue mass of lysis buffer as described in Example 3 and homogenised using a TissueRuptor II (Qiagen). Samples were stored at −20° C. until required. Protein isolation was performed on untreated mice to identify the pathology in the C9orf72 mouse model. Protein isolation is also performed on treated mice to identify effects of CasRx therapy on the pathology.

### Meso Scale Discovery Immunoassay

Mouse brain samples were sonicated at 4° C. for 3×20 seconds at 30% amplitude, and then MSD was used to detect DPR proteins following the method outlined in Example 3.

### RNA-FISH

In a further example, RNA fish is performed on samples from C9orf72 mice (around 12-14 months), and the presence of RNA foci is compared to C9orf72 mice that are treated with the PhP.eB AAV9 vector containing pre-gRNA and CasRx, as outlined in Example 2.

### Statistical Analyses and Data Presentation

Statistical analysis was performed as outlined in Example 3.

### Example 5: CRISPR-Cas13 Systems Can Degrade the Sense C9orf72 Hexanucleotide Repeat Expansion Transcript and Prevent RNA Foci and DPR Formation in a Transient Model

To determine whether Cas13b can be used to target the sense C9orf72 hexanucleotide repeat expansion transcript, we utilised the NanoLuciferase (NLuc) reporter assay as outlined in Example 3. The NLuc reporter plasmid, S92RNL, contains 92 pure G₄C₂ repeats with 120 nucleotides of the endogenous upstream C9orf72 sequence and a NLuc in frame with poly-GR, referred to as S92RNL (FIG. 5A). The S92RNL reporter is a model of RAN translation, which is associated with C9orf72 pathogenesis, as there is no ATG start codon in the plasmid. A control plasmid was used which did not contain any G₄C₂ repeats but was otherwise identical to S92RNL, termed S0RNL.

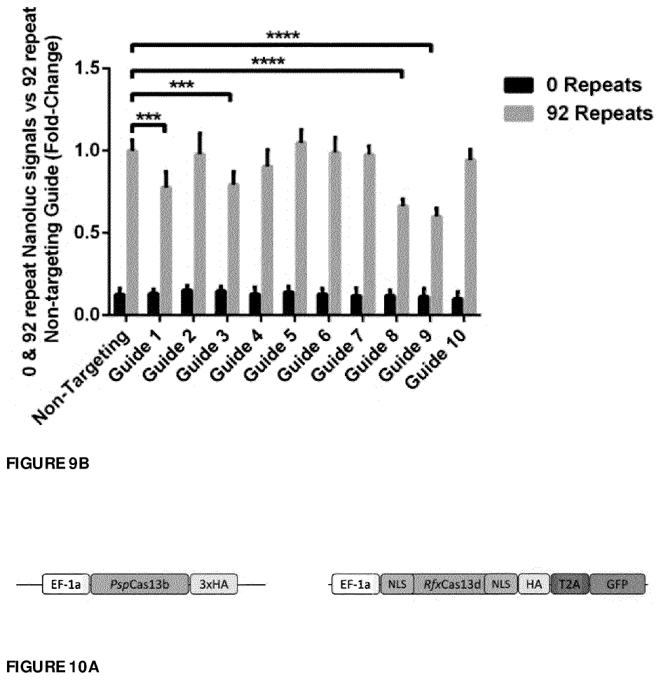

Ten Cas13b 30-nucleotide guides were designed (Example 1) (SEQ ID NOs: 1, 4, 7, 10, 13, 16, 19, 22, 25 and 28) to target the upstream sequence of the C9orf72 sense transcript, and these guides were cloned into the gRNA expressing backbone (detailed in Example 2). The S92RNL or S0RNL reporter plasmid were then co-transfected into HeLa cells along with the Cas13b expressing plasmid, a gRNA expressing plasmid, and a plasmid expressing an ATG-driven Firefly Luciferase (FLuc) to act as a transfection efficiency control (FIG. 9A). 48 hours post-transfection, both the FLuc and NLuc readings were taken and the NLuc signal for each guide was normalised to FLuc per well which was further normalised to a non-targeting control guide (Example 3).

Of the ten guides tested, four achieved a significantly reduced NLuc signal, indicating a significant reduction in poly-GR levels; guide 1 (SEQ ID NO: 1), guide 3 (SEQ ID NO: 7), guide 8 (SEQ ID NO: 22) and guide 9 (SEQ ID NO: 25). Guide 9 (SEQ ID NO: 25) achieved the highest reduction of 40% (±5% S.D.) (FIG. 9B). The lack of an ATG start codon or hexanucleotide repeats necessary for RAN translation in the control S0RNL plasmid resulted in a very low NLuc level, which was taken as the background level of signal.

A new Cas13 ortholog has been discovered, termed Cas13d. Cas13d has also been optimised for efficient transcript knockdown by addition of N-terminal and C-terminal nuclear localisation sequences (NLS), and this variant has been termed CasRx (FIG. 10A; Konermann et al., 2018). Based on the initial data with Cas13b, we then tested whether CasRx can also effectively prevent poly-GR production.

To do this, the initial ten 30-nucleotide guides (SEQ ID NOs: 1, 4, 7, 10, 13, 16, 19, 22, 25 and 28) were cloned into the gRNA expressing backbone (Example 2). We then performed the same S92RNL assay (FIG. 5A) in HEK293T cells to compare CasRx and Cas13b as described in Example 3 (FIG. 10B-D).

These data show that CasRx is more efficient than Cas13b at reducing poly-GR formation in our transient model system, with CasRx reducing the NLuc signal to background levels (FIG. 10B-10D; Table 6). dCasRx did not reduce NLuc levels, suggesting that the binding of CasRx to the transcript is not strong enough to inhibit translation or initiate RNA degradation (FIG. 10E; Table 6).

Interestingly, different guides had different efficiencies depending on the Cas13 ortholog they were used in conjunction with. For Cas13b, guide 5 (SEQ ID NO: 13) was reproducibly the least efficient achieving only a 15% (±6%) reduction in normalised NLuc signal. Whereas guide 5 was very efficient at targeting CasRx to the transcript achieving a 92% (±3%) reduction in normalised NLuc signal. The least efficacious guide with CasRx was guide 3 (SEQ ID NO: 7) (Table 6).

TABLE 6
Comparison of sense targeting guide efficiencies between
Cas13b, CasRx, dCasRx in the S92RNL NanoLuc reporter
assay. Data given as % reduction in NanoLuc signal normalised
to FLuc and non-targeting guide ± standard deviation.

| | | % Reduction in NanoLuc | | |
|---|---|---|---|---|
| | | signal (compared | | |
| | SEQ ID | to non-targeting guide) ± SD | | |
| Guide | NO | Cas13b | CasRx | dCasRx |
| Non- | 77 | 0 ± 4 | 0 ± 7 | 0 ± 9 |
| targeting | | | | |
| 1 | 1 | 72 ± 1 | 99 ± 1 | 10 ± 16 |
| 3 | 7 | 70 ± 5 | 84 ± 4 | 9 ± 11 |
| 5 | 13 | 15 ± 6 | 92 ± 3 | 1 ± 14 |
| 8 | 22 | 75 ± 3 | 99 ± 1 | 2 ± 13 |
| 9 | 25 | 75 ± 1 | 99 ± 1 | 2 ± 13 |
| 10 | 28 | 33 ± 4 | 98 ± 1 | 3 ± 23 |

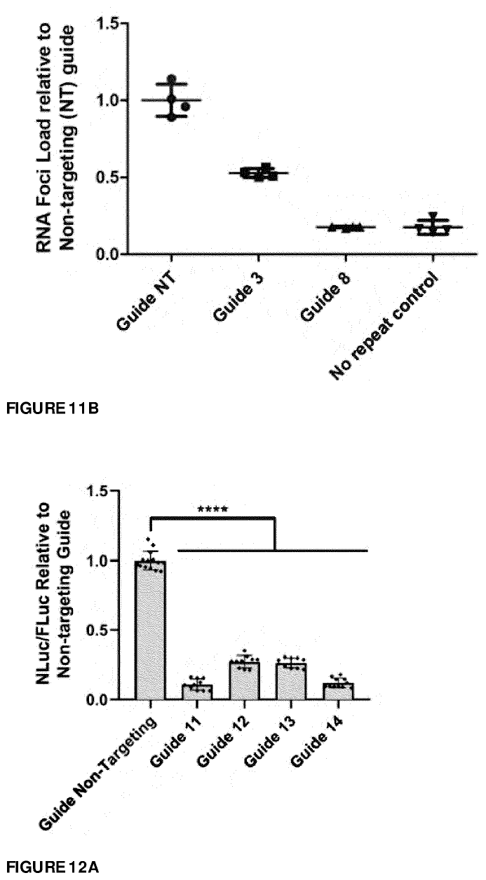

G₄C₂ RNA foci are a pathologic feature of C9orf72 FTD/ALS (Mizielinska et al., 2013). To determine whether CasRx could prevent the formation of RNA foci, the same experimental paradigm was used as described for the S92RNL plasmid. Guides 8 and 3 (SEQ ID NOs: 22 and 7) were selected as representative guides as these guides achieved the highest and lowest NLuc reductions, respectively (Table 6). We then developed a protocol for combined single molecule RNA fluorescent in situ hybridisation (FISH) and ICC to visualise sense RNA foci in CasRx positive cells (see Example 3 for method), as determined by HA positivity. It was not possible to use the GFP signal from CasRx plasmid as the signal is lost during the RNA FISH protocol. Whilst guide 3 (SEQ ID NO: 7) reduced the RNA foci load ˜50%, guide 8 (SEQ ID NO: 22) reduced the foci to background levels as indicated by the no repeat control (FIG. 11A and 11B).

### Example 6: CRISPR-CasRx Targeting of the Antisense C9orf72 Transcript Can Prevent RNA-Foci and DPR Formation in a Transient Model

Antisense C₄G₂ RNA foci and DPRs are also found in C9orf72 patients due to bidirectional transcription of the hexanucleotide repeat sequence and RAN translation of the consequent transcript (Gendron et al., 2013; Mizielinska et al., 2013). Poly-PR is translated from the antisense transcript and has been shown to be one of the most toxic DPRs along with poly-GR, therefore it is imperative for any therapy to also target the antisense transcript (Mizielinska et al., 2014).

We subsequently designed a cloning strategy to produce a NLuc reporter assay for the antisense repeat transcript, with the NLuc in frame with poly-PR and therefore a reporter for poly-PR expression levels. This reporter plasmid contains ˜55 pure C₄G₂ repeats and is termed AS55RNL (FIG. 6A and 6B; Example 2).

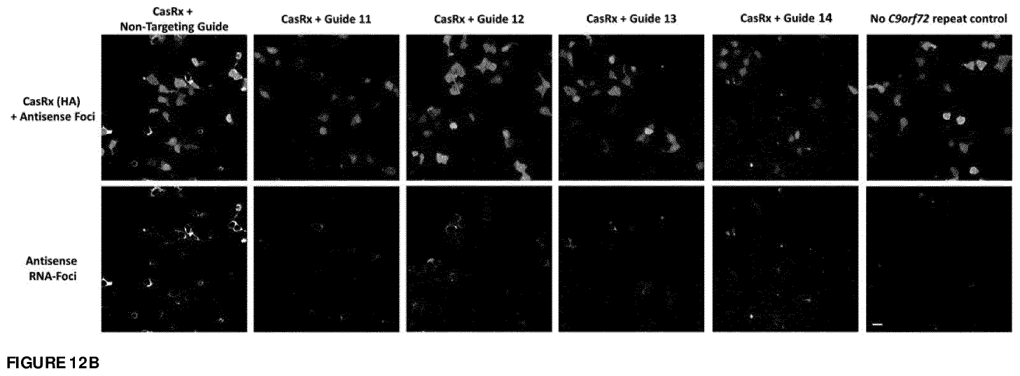

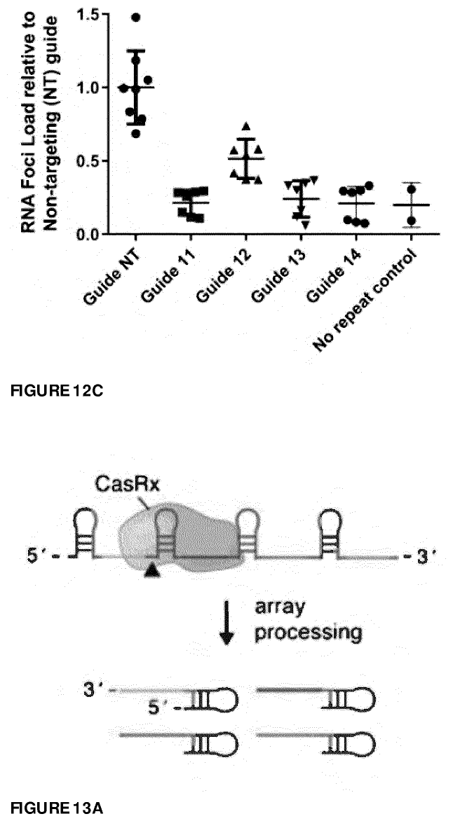

To determine if CasRx can also target the antisense hexanucleotide repeat expansion transcript, guides were designed to target the endogenous sequence 5′ of the repeats (Example 1). Guides with the lowest predicted secondary structure and off-target score (indicated by human transcriptome BLAST) were selected; guides 11-14 (SEQ ID NOs: 31, 34, 37, and 40, respectively) and cloned into the gRNA expressing backbone (Example 2). The CasRx, gRNA (comprising the pre-gRNA+spacer sequence), AS55RNL, and FLuc plasmids were transfected into HEK293T cells and the NLuc and FLuc levels were measured 48 hours later (as in Examples 3 and 5; FIG. 12A). All tested guides reduced the NLuc signal >70% with guide 11 (SEQ ID NO: 31) achieving the greatest reduction of 89% (±4% S.D.). In addition, dual RNA FISH and ICC demonstrated that guides 11 and 14 (SEQ ID NOs: 31 and 40, respectively) reduced the antisense RNA foci to the same level as the no repeat control (as in Examples 3 and 5; FIG. 12B and 12C).

Taken together with the results of Example 6, these data provide promising evidence for the ability of CasRx and specific gRNA combinations to target and degrade both the sense and antisense C9orf72 hexanucleotide repeat expansion transcripts.

### Example 7: CasRx is Efficient with 30 or 22 Nucleotide gRNAs and CasRx Can Mature pre-gRNAs

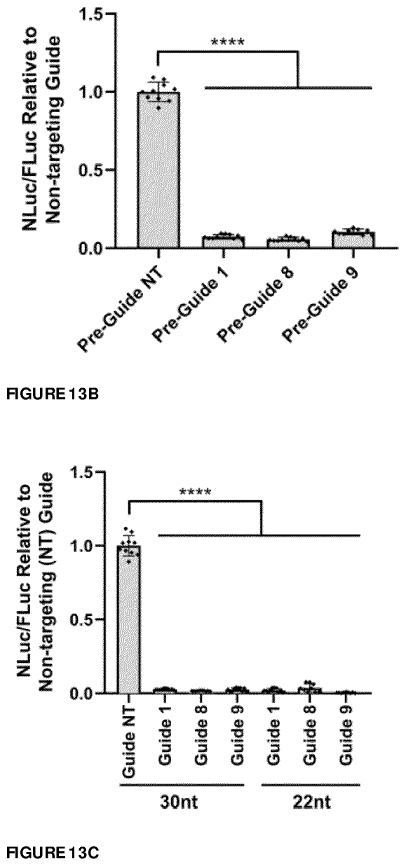

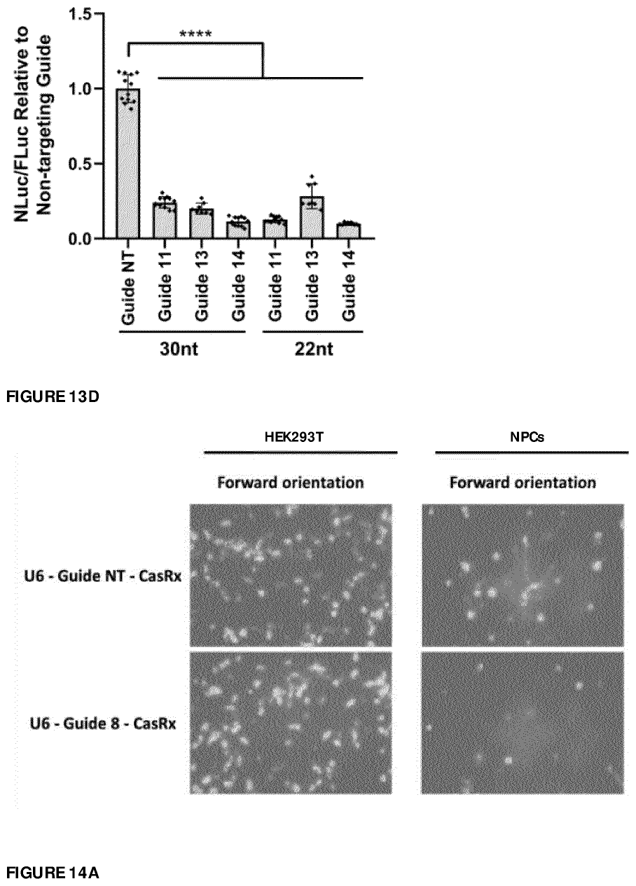

In order to target both sense and antisense transcripts in a single AAV therapy, both sense and antisense guides need to be expressed. CasRx has been shown to mature an immature guide array (i.e., multiple guide RNAs) without additional domains, other enzymes co-expressed, or use of multiple U6 promoters as required by Cas9 (FIG. 13A; Konermann et al., 2018). When CasRx matures pre-gRNAs, it removes ˜8 nucleotides from the 3′ end of the gRNA. However, as this may vary depending on the gRNA, we tested pre-gRNA (and 30nt vs 22nt gRNAs) to determine whether CasRx could mature the specific gRNAs and whether the mature guides were still efficacious at targeting C9orf72 transcripts. As the 5′ 16 nucleotides of the gRNA, closest to the CRISPR direct-repeat, has been shown to be the most important region for guide specificity and efficiency (C. Zhang et al., 2018), the 30 nucleotide guides were truncated to 22 nucleotide guides by removing 8 nucleotides from the 3′ end of the guide sequence.

To determine whether CasRx can mature immature pre-gRNAs into mature gRNAs in our model system and still maintain a high target knockdown efficiency, the guides were cloned into a pre-gRNA expressing plasmid (Addgene, 109054) and tested following the S92RNL NLuc experimental paradigm outlined in Example 3. CasRx was able to mature its own gRNAs and still achieve >95% reduction in poly-GR as indicated by reduce NLuc levels (FIG. 13B) when using pre-gRNA for guides 1, 8 and 9 (SEQ ID NOs: 1, 22 and 25 for spacer sequences and SEQ ID NOs: 2, 23 and 26 for pre-gRNA+spacer sequences, respectively). In addition, both 22 nucleotide and 30 nucleotide gRNAs targeting the sense transcript (FIG. 13C), and the antisense transcript (FIG. 13D) are efficacious at reducing NLuc signals in the S92RNL and AS55RNL assays, respectively. This suggests that when CasRx matures, a mature gRNA length between 22 to 30 nucleotides will successfully knockdown the target transcript.

Taken together, these data suggest that expression of the specific 30-nucleotide pre-gRNAs in an array allows successful targeting and knockdown of the C9orf72 transcript.

### Example 8: Production and Testing of Single Plasmids Expressing gRNA and CasRx

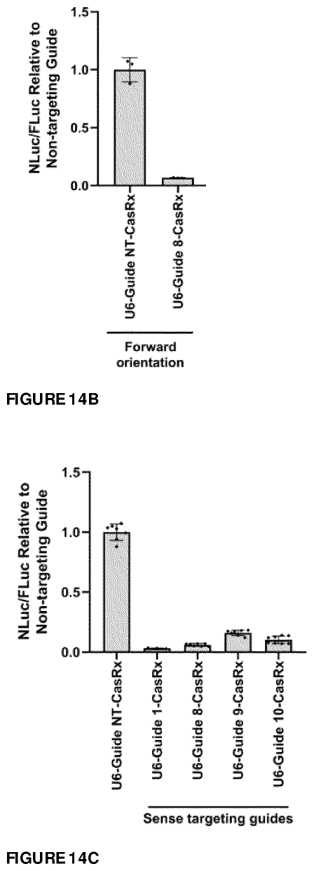

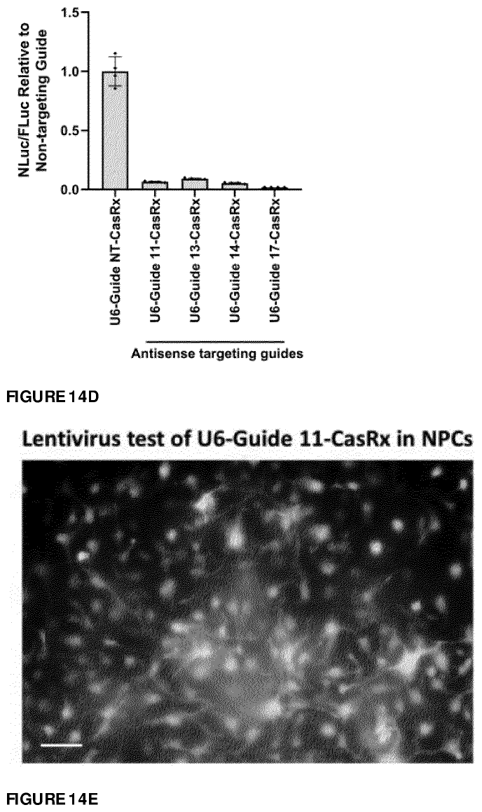

PCR cloning was used to clone the U6 promoter and gRNA sequences (pre-gRNA+spacer sequences) from the guide plasmid into a CasRx-expressing lentiviral vector (as described in Example 2; see FIG. 7). In Cas9 systems that express both gRNA and Cas9 in the same plasmid, it is normal to reverse the orientation of the RNA polymerase III promoter for the gRNA and the RNA polymerase II promoter for the Cas9 with a ˜150nt buffer zone to achieve expression of both gRNA and Cas9. In our cloning strategy, due to use of a single restriction site in the CasRx plasmid, our U6-gRNA fragment will insert in both orientations (FIG. 7). Furthermore, as the resulting plasmid contains a GFP, GFP positive cells should express both gRNA and CasRx. We therefore tested whether ‘forward’ orientations of the plasmids were able to target the sense transcript and reduce NLuc levels using transient transfection into HEK293T cells and iPSC-derived NPCs (FIG. 14A). Indeed, the ‘forward’ orientation of guide 8 was able to target the sense transcript and reduce NLuc levels (FIG. 14B).

Therefore, all ‘forward’ orientation single gRNA CasRx expressing plasmids targeting both sense and antisense transcripts were then tested in the S92RNL and AS55RNL NLuc assays, respectively and were found to effectively target their respective transcripts in HEK293T cells (FIG. 14C and 14D). This promising result indicates that at a ratio of 1:1 CasRx to gRNA leads to an effective knockdown (FIG. 14C and 14D). In the previous NanoLuc assays utilising separate gRNA and CasRx expressing plasmids a molar ratio of 5:1 gRNA to CasRx was used (FIGS. 10C and 12A). The antisense gRNAs seemed to be less efficient compared with the sense targeting gRNAs with CasRx (˜89% knockdown for antisense vs ˜99% knockdown for sense with most efficient guides). This is unsurprising as the 200-nucleotide sequence 5′ of the repeats that is targeted to reduce antisense transcripts is ˜80% GC-rich. This likely reduces guide binding efficiency and increases RNA secondary structure, further restricting access to target sites for the gRNA-CasRx complex. However, Guide 17 is a new antisense-targeting gRNA that targets a sequence further from the repeats (>200 bp from 5′ end of repeat sequence in the C9orf72 antisense strand) than the other antisense guides previously tested (<200 bp from 5′ end of the repeat sequence in the C9orf72 antisense strand) where the sequence is less GC-rich. Guide 17 (SEQ ID NO: 43, pre-gRNA+spacer SEQ ID NO: 44) was very efficient in this assay and reduced poly-PR to background levels (FIG. 14D) and appears to be the most efficient antisense-targeting gRNA, with a NLuc signal knockdown of 99% (±2% S.D.).

### Example 9: Testing CasRx and Guide-RNAs in iPSC-Derived NPCs

The NLuc transient assays model the endogenous C9orf72 expanded sense and antisense transcripts by containing pure sense or antisense repeats, no ATG start codon, and a portion of the endogenous sequence 5′ or 3′ of the repeat. However, in order to determine whether the gRNAs and CasRx successfully target endogenous C9orf72 transcripts to reduce pathology without affecting variant 2 of C9orf72 or hitting off-target transcripts, the gRNA and CasRx therapy is tested in iPSC-derived neuronal progenitor cells (NPCs) which endogenously express C9orf72 transcripts.

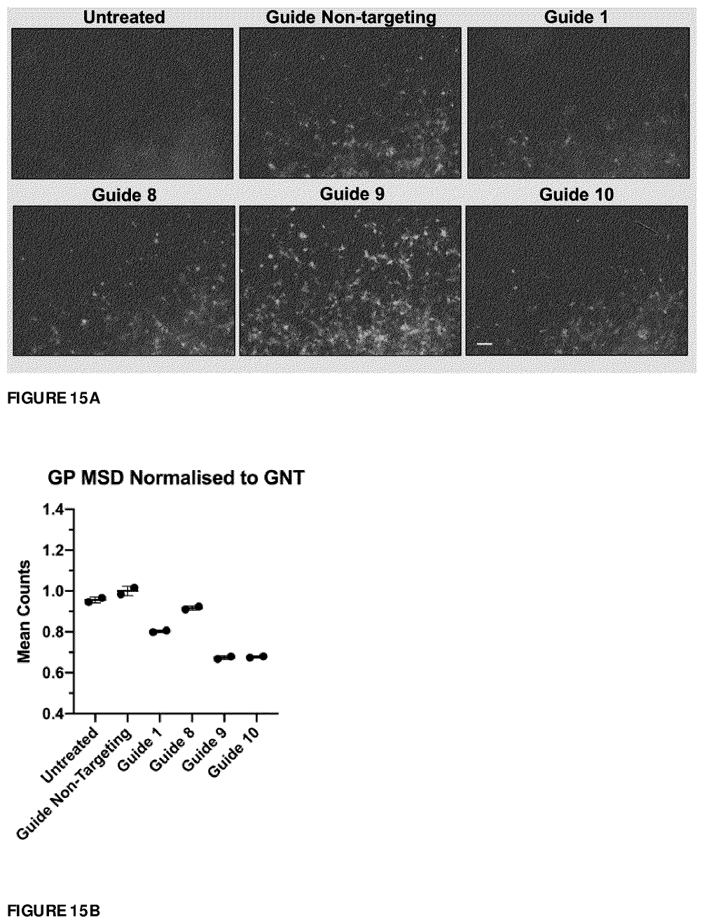

The U6 promoter and gRNA sequences were cloned into a CasRx-expressing lentiviral vector as described in Example 2 and used to produce CasRx and gRNA-expressing lentiviruses as described in Example 3. iPSC-derived NPCs were then transduced as described in Example 3, and MSD performed (Example 3). The CasRx and gRNA expressing lentiviral plasmid comprises an eGFP tag which allows visualisation of transduction efficiency. As seen in FIG. 15A, transduction in this case was achieved in 30% of the of iPSC-derived NPCs. However, despite the low transduction efficiency, expression of CasRx in combination with gRNA with guides 9 (SEQ ID NO: 25), or 10 (SEQ ID NO: 28) significantly reduced the counts of Poly-GA in iPSC-derived NPCs (FIG. 15B). In addition, although CasRx in combination with guide RNAs 1 (SEQ ID NO: 2) or 8 (SEQ ID NO: 23) did not appear to significantly reduce the counts of Poly-GA in iPSC-derived NPCs, FIG. 15B demonstrates that there is a general trend of reducing the number of Poly-GP counts. Therefore, it is expected that following optimisation of the transduction protocol, guide RNAs 1 (SEQ ID NO: 2) or 8 (SEQ ID NO: 23) will also significantly reduce the number of Poly-GP counts in iPSC-derived NPCs.

The effect of CasRx and guide RNAs in iPSC-derived neurons is detailed in Example 12.

In this Example, additional tests are performed to determine whether CasRx from the lentiviruses can target the endogenous C9orf72 transcripts to reduce RNA-foci and DPRs without reducing variant 2 of C9orf72 or hitting off-target transcripts. To determine whether the gRNAs result in off-target transcript changes, RNA-sequencing is performed on patient-derived cells transduced with gRNA-CasRx expressing lentiviruses.

### Example 10: CasRx AAV Treatment for C9orf72 BAC Mouse Model

In a further Example, a single AAV therapy comprising CasRx and the presently disclosed gRNA (as described in Example 2) is used to determine whether CasRx AAV therapy can reverse C9orf72 pathology in the C9orf72 bacterial artificial chromosome (BAC) mouse model (described in Example 4). As outlined in Example 2, the PhP.eB AAV backbone is used which has been previously demonstrated to have high CNS transduction efficiency and expression in certain mouse backgrounds (Chan et al. (2017). Engineered AAVs for efficient noninvasive gene delivery to the central and peripheral nervous systems. Nature Neuroscience, 20(8), 1172-1179. https://doi.org/10.1038/nn.4593). The PhP.eB AAV backbone contains a Gateway cloning site to facilitate cloning in of the sense and antisense transcript targeting gRNAs.

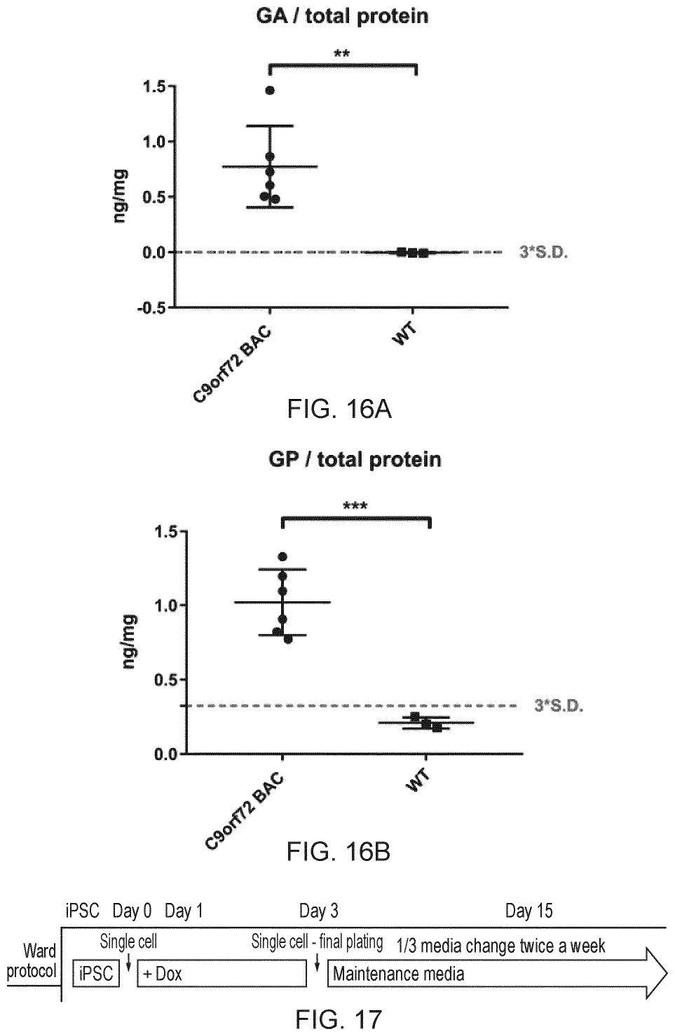

At 3 months of age, C9orf72 BAC mice show detectable levels of poly-GA and poly-GP, but not poly-PR or poly-GR (FIG. 16A and 16B respectively). In further Examples, 12 month old C9orf72 BAC mice are also expected to show detectable levels of poly-GA and poly-GP, along with detectable poly-PR or poly-GR.

Additionally, samples from 3 or 12 month old C9orf72 BAC mice and their controls are analysed using RNA-FISH to demonstrate RNA foci pathology.

Young, or aged (˜3 months and ˜12-14 months, respectively) C9orf72 BAC mice are treated with a single AAV expressing CasRx and both sense and antisense transcript-targeting guide RNAs (Example 2). Treated mice are then analysed by MSD or RNA FISH used to determine improvements in C9orf72 pathology such as DPRs and RNA foci, respectively. The data are expected to show that CasRx and gRNA combinations can reverse the established pathological features of C9orf72.

In a further Example, an alternative mouse model is used which expresses G₄C₂ repeats via an AAV and exhibits both sense and antisense pathology. In these experiments, the mouse model and controls are tested for DPRs, RNA foci, as outlined for the C9orf72 BAC mice above, and behavioural/motor phenotypes. The mice are then treated with the described AAV therapy and tested for any therapeutic effect on the pathology or delay of symptomatic onset as outlined above.

### Example 11: Effect of CasRx AAV Therapy on Immune Response

A concern with the CasRx strategy is the potential for triggering an immune response in the host organism to the CasRx. It has previously been shown that some patients already possess antibodies to certain Cas9 orthologs (Charlesworth et al. (2019). Identification of preexisting adaptive immunity to Cas9 proteins in humans. Nature Medicine, 25(2), 249-254. https://doi.org/10.1038/s41591-018-0326-x), although the immune response does not trigger extensive cell damage in vivo (Chew et al. (2016). A multifunctional AAV-CRISPR-Cas9 and its host response. Nature Methods, 13(10), 868-874. https://doi.org/10.1038/nmeth.3993). However, it has not yet been confirmed whether the immune response reduces Cas9 efficacy. Cas13research is comparatively in its infancy and it is yet to be determined whether the human immune system could mount anti-Cas13 responses. There is a precedent from previous gene therapies which have overcome T-cell responses to capsids and transgenes that suggest these issues are all surmountable with well-designed and tested vectors, promoters, administration methods, and immune suppression (Shirley et al. (2020). Immune Responses to Viral Gene Therapy Vectors. Molecular Therapy, 28(3), 709-722. https://doi.org/10.1016/j.ymthe.2020.01.001). Therefore, in this Example, the immune responses of the C9orf72 mouse models of Example 10 are monitored, and animals treated with the AAV therapy are tested for antibodies against CasRx using well known assays.

All publications mentioned in the above specification are herein incorporated by reference. Various modifications and variations of the described methods, uses and products of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

### Example 12: Testing CasRx and Guide-RNAs in iPSC-Derived Neurons

### Methods

### i3 Neuron Differentiation and Transfection with CasRx Lentiviruses

iPSC were transfected (LipoStem) with a piggyBac vector expressing Doxycycline-inducible hNGN2 (Fernandopulle et al. 2018. Transcription Factor-Mediated Differentiation of Human iPSCs into Neurons. Curr Protoc Cell Biol. 79(1):e51. doi: 10.1002/cpcb.51). Cells stably expressing the piggyBac vector were selected via fluorescence activated cell sorting (FACS). Stable i3 neuron lines were generated for 3 patient/isogenic pairs (Table 1).

Cells could then be rapidly differentiated into i3 cortical neurons as previously described (Fernandopulle et al. 2018). The method of differentiation is outlined in FIG. 17. In brief, iPSCs are dissociated with accutase prior to plating on Geltrex-coated plates in induction media (DMEM, N2 supplement, non-essential amino acids, GlutaMAX, HEPES, 2 ug/ml Doxycycline, ROCK inhibitor). 2 hours after plating, cells are transduced with lentiviruses expressing CasRx and various guide RNAs (in a single lentivirus as outlined in Examples 2 and 3). Fresh media was supplied once per day. 3 days post induction cells were changed to Maintenance media (Neurobasal, B27 supplement, BDNF 10 ng/ml, NT-3 10 ng/ml, Laminin) until FACS on day 5 post-induction.

### Flourescence-Activated Cell Sorting (FACS)

Cells were prepared for FACS via accutase dissociation followed by suspension in sorting buffer (1% BSA, 2 mM EDTA in PBS). Dissociated cells were then strained through a 100 μm cell strainer to remove any cell clumps.

The filtered cells were then sorted for GFP (present in the CasRx lentiviruses) using a BD FACSAria (BD Biosciences) cell sorter. Cells positive for GFP above background (set with non-transduced control samples) were collected for downstream analyses.

### RNA Extraction and qPCR

RNeasy kits (Qiagen) were used to isolate RNA according to manufacturer's protocol. 1 μg of RNA was then reverse transcribed following DNase treatment using SuperScript® IV Reverse Transcriptase kit (Invitrogen). RNA levels were analysed via PCR using SYBR Power-Up Master Mix (ThermoFisher, Massachusetts, USA) and the QuantStudio Flex system (Applied Biosystems, ThermoFisher, USA); temperatures and cycles were altered according to Tₘ of primers used. The primers used are outlined in Table 7. All primers were sourced from Sigma. Data was double-normalized to both house-keeping genes and displayed as fold-change compared to control using the 2^{-ΔΔCt} method.

TABLE 7
Primers

| Primers (qPCR) | SEQ ID NO | Sequence (5′-3′) |
|---|---|---|
| Repeat containing intron 1a Fwd | 67 | CCCCACTACTTGCTCTCACA |
| Repeat containing intron 1a Rev | 68 | CGGTTGTTTCCCTCCTTGTT |
| Exon 1b containing Fwd | 69 | GCGGTGGCGAGTGGATAT |
| Exon 1b containing Rev | 70 | TGGGCAAAGAGTCGACATCA |
| Antisense repeat transcript Fwd | 71 | AGTCGCTAGAGGCGAAAGC |
| Antisense repeat containing Rev | 72 | CGAGTGGGTGAGTGAGGAG |
| GAPDH Fwd | 73 | GTCTCCTCTGACTTCAACAGCG |
| GAPDH Rev | 74 | ACCACCCTGTTGCTGTAGCCAA |

### Meso Scale Discovery (MSD) Immunoassay

Protein was extracted, and MSD analysis performed as described in Example 3.

### Results

In order to determine whether the endogenous C9orf72 transcripts can be targeted and pathology reduced, we utilized patient iPSC-derived cortical neurons, termed i3 neurons, transduced with lentiviruses expressing CasRx and either one targeting guide RNA (guide 8 (pre-gRNA+spacer SEQ ID NO: 23) or guide 10 (pre-gRNA+spacer SEQ ID NO: 29)), or a control non-targeting guide RNA (SEQ ID NO: 77) as described above. 5 days post differentiation and transduction, i3 neurons underwent fluorescence-activated cell sorting (FACS) to purify cells transduced with our lentiviruses. Protein and RNA was extracted for downstream analysis, as described herein.

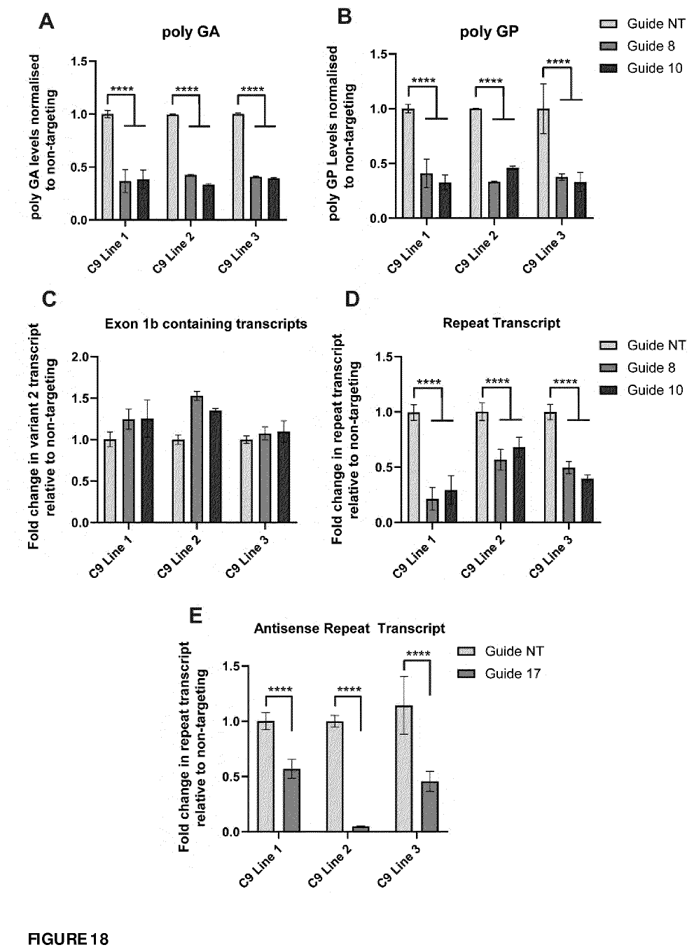

5 days after transduction with the CRISPR-CasRx lentiviruses targeting the endogenous C9orf72 repeat expansion RNA, there was a significant reduction of around 55-70% in both poly-GA and poly-GP in the iPSC-derived neurons, two pathological DPR hallmarks of C9orf72 FTD/ALS, as indicated by meso-scale discovery (MSD) immunoassays (FIG. 18A and 18B; methods of performing MSD immunoassays are described in Example 3). This reduction was observed when using either guide 8 and guide 10, and the reduction was observed in 3 different patient lines with 3 separate inductions performed for each line. qPCR analysis of the C9orf72 transcripts in these samples reveal a reduction in pathogenic repeat-containing transcripts, but exon 1b containing transcripts that do not contain the hexanucleotide repeat expansion are spared (FIG. 18C and 18D). It is important the exon 1b containing transcripts are spared to prevent further loss of functional, long isoform C9orf72 protein. Additionally, we also observed a significant reduction in the anti-sense repeat-containing transcript with guide 17 (pre-gRNA+spacer SEQ ID NO: 44) (FIG. 18E).

This data confirms that CRISPR-CasRx can target endogenous repeat-containing sense and antisense C9orf72 transcripts in patient iPSC-derived neurons and reduce C9orf72 repeat-associated pathology in patient-derived iPSC neurons.

### Example 13: CRISPR-CasRx Delivered via AAV Can Target C9orf72 Transcripts In Vivo

### Methods

### Animals

Animals were maintained and experimental procedures performed in accordance with the UK Animal Scientific Procedure Act 1986, under project and personal licenses issued by the UK Home Office. All mice used in this study were of the C57b16/J strain (RRID: IMSR_JAX: 000664) and were maintained in individually ventilated cages. For tissue collection, animals were anaesthetized with isoflurane and perfused with ice-cold PBS. Brains were immediately collected, dissected, and snap-frozen on dry ice. All brain tissues used in this study were collected at postnatal day 22 or 23 (P22-23).

### Neonatal Intracerebroventricular (ICV) Injections

AAVs used for ICVs were generated and purified by the Viral Vector Facility at ETH Zurich according to previously published protocols (Chan et al. 2017).

A C9orf72 AAV mouse model was produced comprising 149 hexanucleotide repeats and a portion of the endogenous upstream and downstream endogenous sequence of C9orf72 (AAV: 149R in Table 8). The C9orf72 mouse model was generated by injection at P0 with the 149R AAV of Chew et al. At P0, mice were also injected with either a CasRx AAV containing Guides 10 and 17 ((pre-gRNA+spacer SEQ ID NOs: 29 and 44, respectively; AAV referred to as AAV9::CasRx.g10.17), or a CasRx AAV containing a non-targeting guide (SEQ ID NO: 77, AAV referred to as AAV9::CasRx.gNT). The AAV9::CasRx.g10.17 and AAV9::CasRx.gNT were generated as described in Examples 2 and 3. Viruses and doses used in this study are outlined in Table 8.

TABLE 8
Viruses and doses used for neonatal ICV injections

| AAV | Viral genomes (vg) per animal |
|---|---|
| AAV9::149R (Chew et al., 2019*) | 6.00E ± 10 |
| AAV9::CasRx.gNT | 8.00E ± 09 |
| AAV9::CasRX.g10.17 | 8.00E ± 09 |

*Chew et al. (2019. Aberrant deposition of stress granule-resident proteins linked to C9orf72-associated TDP-43 proteinopathy. Mol Neurodegener, 14(1): 9. doi: 10.1186/s13024-019-0310-z).

For generating postnatal day 0 (P0) pups, pregnant females were individually housed and checked daily for new litters. Within 24 hr of birth, pups were manually injected with adeno associated viruses (AAV) serotype 9 via intracerebroventricular (ICV) injection into both hemispheres. Briefly, AAVs were diluted in sterile PBS to a final volume of 5 μL per animal. P0 pups were anaesthetized with isoflurane, after which a calibrated Hamilton 10 μL syringe was inserted into the skull approximately ⅖ of the distance from lambda to the eye and at a depth of approximately 2 mm. 2.5 μL of AAV/PBS solution was injected slowly into each hemisphere. After injection, pups were allowed to recover on a heat pad and then returned to the dam.

### RNA Extraction and qPCR

The mice were then culled 3 weeks after injection and RNA extracted from the hippocampus and reverse transcribed using the same techniques outlined in Example 12. qPCR was performed for the 149R AAV repeat-containing RNA using SYBR Power-Up Master Mix (TermoFisher, Massachusetts, USA) and the QuantStudio Flex system (Applied Biosystems, TermoFisher, USA); temperatures and cycles were altered according to Tₘ of primers used. The primers used are outlined in Table 9. All primers were sourced from Sigma. Data was double-normalized to both house-keeping genes and displayed as fold-change compared to control using the 2^{-ΔΔCt} method.

TABLE 9
Primers

| Primers (qPCR) | SEQ ID NO | Sequence (5′-3′) |
|---|---|---|
| 149R AAV repeats Fwd | 75 | AGTACTCGCTGAGGGTGAACAAG |
| 149R AAV repeats Rev | 76 | AGCTTGCTACAGGCTGCGGTTG |
| GAPDH Fwd | 73 | GTCTCCTCTGACTTCAACAGCG |
| GAPDH Rev | 74 | ACCACCCTGTTGCTGTAGCCAA |

### Results

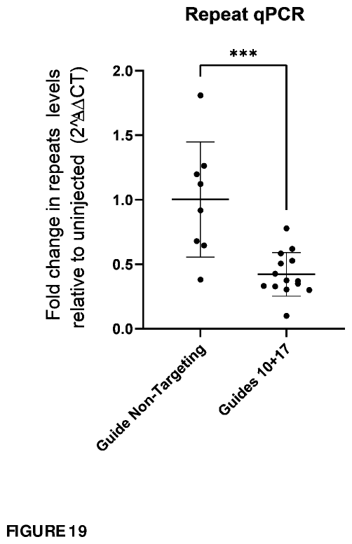

There was ˜50% reduction of the repeat-containing transcript after 3 weeks in mice injected with the CasRx AAV expressing targeting guides 10 and 17, compared with mice injected with control CasRx AAV expressing non-targeting guide RNA (SEQ ID NO: 77) (FIG. 19). This data demonstrates that CRISPR-CasRx AAV can reduce C9orf72 repeat-containing RNA in vivo.

In further examples, the C9orf72 mouse model is analysed to determine whether treatment with the CRISPR-CasRx AAV plasmid results in an improvement in the onset of FLS/ATD behavioural/motor symptoms in mice aged 3 and 12 months as compared to controls.

| Nucleotide sequence of C9orf72 gene | |
|---|---|
| Lowercase letters indicate repetitive sequences or sequences that commonly vary. | |
| SEQ ID NO: 56 | |
| 1 | ACGTAACCTA CGGTGTCCCG CTAGGAAAGA GAGGTGCGTC AAACAGCGAC AAGTTCCGCC |
| 61 | CACGTAAAAG ATGACGCTTG GTGTGTCAGC CGTCCCTGCT GCCCGGTTGC TTCTCTTTTG |
| 121 | GGGGCGGGGT CTAGCAAGAG CAGGTGTGGG TTTAGGAGGT GTGTGTTTTT GTTTTTCCCA |
| 181 | CCCTCTCTCC CCACTACTTG CTCTCACAGT ACTCGCTGAG GGTGAACAAG AAAAGACCTG |
| 241 | ATAAAGATTA ACCAGAAGAA AACAAGGAGG GAAACAACCG CAGCCTGTAG CAAGCTCTGG |
| 301 | AACTCAGGAG TCGCGCGCTA ggggccgggg ccggggccgg ggcgtggtcg gggcgggccc |
| 361 | gggggcgggc ccggggcggg gcTGCGGTTG CGGTGCCTGC GCCCGCGGCG GCGGAGGCGC |
| 421 | AGGCGGTGGC GAGTGGGTGA GTGAGGAGGC GGCATCCTGG CGGGTGGCTG TTTGGGGTTC |
| 481 | GGCTGCCGGG AAGAGGCGCG GGTAGAAGCG GGGGCTCTCC TCAGAGCTCG ACGCATTTTT |
| 541 | ACTTTCCCTC TCATTTCTCT GACCGAAGCT GGGTGTCGGG CTTTCGCCTC TAGCGACTGG |
| 601 | TGGAATTGCC TGCATCCGGG CCCCGGGCTT CCcggcggcg gcggcggcgg cggcggcgCA |
| 661 | GGGACAAGGG ATGGGGATCT GGCCTCTTCC TTGCTTTCCC GCCCTCAGTA CCCGAGCTGT |
| 721 | CTCCTTCCCG GGGACCCGCT GGGAGCGCTG CCGCTGCGGG CTCGAGAAAA GGGAGCCTCG |
| 781 | GGTACTGAGA GGCCTCGCCT GGGGGAAGGC CGGAGGGTGG GCGGCGCGCG GCTTCTGCGG |
| 841 | ACCAAGTCGG GGTTCGCTAG GAACCCGAGA CGGTCCCTGC CGGCGAGGAG ATCATGCGGG |
| 901 | ATGAGATGGG GGTGTGGAGA CGCCTGCACA ATTTCAGCCC AAGCTTCTAG AGAGTGGTGA |
| 961 | TGACTTGCAT ATGAGGGCAG CAATGCAAGT CGGTGTGCTC CCCATTCTGT GGGACATGAC |
| 1021 | CTGGTTGCTT CACAGCTCCG AGATGACACA GACTTGCTTA AAGGAAGTGA CTATTGTGAC |
| 1081 | TTGGGCATCA CTTGACTGAT GGTAATCAGT TGTCTAAAGA AGTGCACAGA TTACATGTCC |
| 1141 | GTGTGCTCAT TGGGTCTATC TGGCCGCGTT GAACACCACC AGGCTTTGTA TTCAGAAACA |
| 1201 | GGAGGGAGGT CCTGCACTTT CCCAGGAGGG GTGGCCCTTT CAGATGCAat cgagattgtt |
| 1261 | aggctctggg agagtagttg cctggttgtg gcagttggta aatttctatt caaacagttg |
| 1321 | ccatgcacca gttgttcaca acaagggtac gtaatctgtc tggcattact tctacttttg |
| 1381 | tacaaaggat caaaaaaaaa aaagatactg ttaagatatg atttttctca gactttggga |
| 1441 | aacttttaac ataatctgtg aatatcacag aaacaagact atcatatagg GGATATTAAT |
| 1501 | AACCTGGAGT CAGAATACTT GAAATACGGT GTCATTTGAC ACGGGCATTG TIGTCACCAC |
| 1561 | CTCTGCCAAG GCCTGCCACT TTAGGAAAAC CCTGAATCAG TTGGAAACTG CTACATGCTG |
| 1621 | ATAGTACATC TGAAACAAGA ACGAGAGTAA TTACCACATT CCAGATTGTT CACTAAGCCA |
| 1681 | GCATTTACCT GCTCCAGGAA AAAATTACAA GCACCTTATG AAGTTGATAA AATATTTTGT |
| 1741 | TTGGCTATGT TGGCACTCCA CAATTTGCTT TCAGAGAAAC AAAGTAAACC AAGGAGGACT |
| 1801 | TCTGTTTTTC AAGTCTGCCC TCGGGTTCTA TTCTACGTTA ATTAGATAGT TCCCAGGAGG |
| 1861 | ACTAGGTTAG CCTACCTATT GTCTGAGAAA CTTGGAACTG TGAGAAATGG CCAGATAGTG |
| 1921 | ATATGAACTT CACCTTCCAG TCTTCCCTGA TGTTGAAGAT TGAGAAAGTG TTGTGAACTT |
| 1981 | TCTGGTACTG TAAACAGTTC ACTGTCCTTG AAGTGGTCCT GGGCAGCTCC TGTTGTGGAA |
| 2041 | AGTGGACGGT TTAGGATCCT GCTTCTCTTT GGGCTGGGAG AAAATAAACA GCATGGTTAC |
| 2101 | AAGTATTGAG AGCCAGGTTG GAGAAggtgg cttacacctg taatgccaga gctttgggag |
| 2161 | gcggaggcaa gaggatcact tgaagccagg agttcaagct caacctgggc aacgtagacc |
| 2221 | ctgtctctac aaaaaattaa aaacttagcc gggcgtggtg atgtgcacct gtagtcctag |
| 2281 | ctacttggga ggctgaggca ggagggtcat ttgagcccaa gagtttgaag ttaccgagag |
| 2341 | ctatgatcct gccagtgcat tccagcctgg atgacaaaac gagaccctgt ctctaaaaaa |
| 2401 | caagaaGTGA GGGCTTTATG ATTGTAGAAT TTTCACTaca atagcagtgg accaaccacc |
| 2461 | tttctaaata ccaatcaggg aagagatggt tgatttttta acagacgttt aaagaaaaag |
| 2521 | caaaacctca aacttagcac tctactaaca gttttagcag atgttaatta atgtaatcat |
| 2581 | gtctgcatgt atgGGATTAT TTCCAGAAAG TGTATTGGGA AACCTCTCAT GAACCCTGTG |
| 2641 | AGCAAGCCAC CGTCTCACTC AATTTGAATC TTGGCTTCCC TCAAAAGACT GGCTAATGTT |
| 2701 | TGGTAACTCT CTGGAGTAGA CAGCACTACA TGTACGTAAG ATAGGTACAT AAACAACTAT |
| 2761 | TGGTTTTGAG CTGATTTTTT TCAGCTGCAT TTGCATGTAT GGATTTTTCT CACCAAAGAC |
| 2821 | GATGACTTCA AGTATTAGTA AAATAATTGT ACAGCTCTCC TGATTATACT TCTCTGTGAC |
| 2881 | ATTTCATTTC CCAGGCTATT TCTTTTGGTA GGATTTAAAA CTAAGCAATT CAGTATGATC |
| 2941 | TTTGTCCTTC ATTTTCTTTC TTATTCtttt tgtttgtttg tttgtttgtt tttttcttga |
| 3001 | ggcagagtct ctctctgtcg cccaggctgg agtgcagtgg cgccatctca gctcattgca |
| 3061 | acctctgcca cctccgggtt caagagattc tcctgcctca gcctcccgag tagctgggat |
| 3121 | tacaggtgtc caccaccaca cccggctaat tttttgtatt tttagtagag gtggggtttc |
| 3181 | accatgttgg ccaggctggt cttgagctcc tgacctcagg tgatccacct gcctcggcct |
| 3241 | accaaagagc tgggataaca ggtgtgaccc accatgcccg gccCAttttt tttttCTTAT |
| 3301 | TCTGTTAGGA GTGAGAGTGT AACTAGCAGT ATAATAGTTC AATTTTCACA ACGTGGTAAA |
| 3361 | AGTTTCCCTA TAATTCAATC AGATTTTGCT CCAGGGTTCA GTTCTGTTTT AGGAAATACT |
| 3421 | TTTATTTTCA GTTTAATGAT GAAATATTAG AGTTGTAATA TTGCCTTTAT GATTATCCAC |
| 3481 | CTTTTTAACC TAAAAGAATG AAAGAAAAAT ATGTTTGCAA TATAATTTTA TGGTTGTATG |
| 3541 | TTAACTTAAT TCATTATGTT GGCCTCCAGT TTGCTGTTGT TAGTTATGAC AGCAGTAGTG |
| 3601 | TCATTACCAT TTCAATTCAG ATTACATTCC TATATTTGAT CATTGTAAAC TGACTGCTTA |
| 3661 | CATTGTATTA AAAACAGTGG ATATTTTAAA GAAGCTGTAC GGCTTATATC TAGTGCTGTC |
| 3721 | TCTTAAGACT ATTAAATTGA TACAACATAT TTAAAAGTAA ATATTACCTA AATGAATTTT |
| 3781 | TGAAATTACA AATACACGTG TTAAAACTGT CGTTGTGTTC AACCATTTCT GTACATACTT |
| 3841 | AGAGTTAACT GTTTTGCCAG GCTCTGTATG CCTACTCATA ATATGATAAA AGCACTCATC |
| 3901 | TAATGCTCTG TAAATAGAAG TCAGTGCTTT CCATCAGACT GAACTCTCTT GACAAGATGT |
| 3961 | GGATGAAATT CTTTAAGTAA AATTGTTTAC TTTGTCATAC ATTTACAGAT CAAATGTTAG |
| 4021 | CTCCCAAAGC AATCATATGG CAAAGATAGG TATATCATAG TTTGCCTATT AGCTGCTTTG |
| 4081 | TATTGCTATT ATTATAAATA GACTTCACAG TTTTAGACTT GCTTAGGTGA AATTGCAATT |
| 4141 | CTTTTTACTT TCAGTCTTAG ATAACAAGTC TTCAATTATA GTACAATCAC acattgctta |
| 4201 | ggaatgcatc attaggcgat tttgtcatta tgcaaacatc atagagtgta cttacacaaa |
| 4261 | cctagatagt atagccttta tgtacctagg ccgtatggta tagtctgttg ctcctaggcc |
| 4321 | acaaacctgt acaactgtta ctgtactgaa tactatagac agttgtaaca cagtggtaaa |
| 4381 | tatttatcta aatatatgca aacagagaaa aggtacagta aaagtatggt ataaaagata |
| 4441 | atggtatacc tgtgtaggcc acttaccacg aatggagctt gcaggactag aagttgctct |
| 4501 | gggtgagtca gtgagtgagt ggtgaattaa tgtgaaggcc tagaacactg tacaccactg |
| 4561 | tagactataa acacagtacg ctgaagctac accaaattta tottaacagt ttttcttcaa |
| 4621 | taaaaaatta taacttttta actttgtaaa ctttttaatt ttttaacttt taaaatactt |
| 4681 | agcttgaaac acaaatacat tgtatagcta tacaaaaata ttttttcttt gtatccttat |
| 4741 | tctagaagct tttttctatt ttctatttta aatttttttt tttacttgtt agtcgttttt |
| 4801 | gttaaaaact aaaacacaca cactttcacc taggcataga caggattagg atcatcagta |
| 4861 | tcactccctt ccacctcact gccttccacc tccacatctt gtcccactgg aaggttttta |
| 4921 | ggggcaataa cacacatgta gctgtcacct atgataacag tgctttctgt tgaatacctc |
| 4981 | ctgaaggact tgcctgaggc tgttttacat ttaacttaaa aaaaaaaaaa gtagaaggag |
| 5041 | tgcactctaa aataacaata aaaggcatag tatagtgaat acataaacca gcaatgtagt |
| 5101 | agtttattat caagtgttgt acactgtaat aattgtatgt gctatacttt aaataacttg |
| 5161 | caaaatagta ctaagacctt atgatggtta cagtgtcact aaggcaatag catattttca |
| 5221 | ggtccattgt aatctaatgg gactaccatc atatatgcag tctaccattg actgaaacgt |
| 5281 | tacatggcac ataactgTAT TTGCAAGAAT GATTTGTTTT ACATTAATAT CACATAGGAT |
| 5341 | GTACCTTTTT AGAGTGGTAT GTTTATGTGG ATTAAGATGT ACAAGTTGAG CAAGGGGACC |
| 5401 | AAGAGCCCTG GGTTCTGTCT TGGATGTGAG CGTTTATGTT CTTCTCCTCA TGTCTGTTTT |
| 5461 | CTCATTAAAT TCAAAGGCTT GAACGGGCCC TATTTAGCCC TTCTGTTTTC TACGTGTTCT |
| 5521 | AAATAactaa agcttttaaa ttctagccat ttagtgtaga actctctttg cagtgatgaa |
| 5581 | atgctgtatt ggtttcttgg ctagcatatt aaatattttt atctttgtct tgatacttca |
| 5641 | atgtcgtttt aaacatcagg atcgggcttc agtattctca taaccagaga gttcactgag |
| 5701 | gatacaggac tgtttgccca ttttttgtta tggctccaga cttgtggtat ttccatgtct |
| 5761 | tttttttttt tttttttttt gaccttttag cggctttaaa gtatttctgt tgttaggtgt |
| 5821 | tgtattactt ttctaagatt acttaacaaa gcaccacaaa ctgagtggct ttaaacaaca |
| 5881 | gcaatttatt ctctcacaat tctagaagct agaagtccga aatcaaagtg ttgacagggg |
| 5941 | catgatcttc aagagagaag actctttcct tgcctcttcc tggcttctgg tggttaccag |
| 6001 | caatcctgag tgttcctttc ttgccttgta gtttcaacaa tccagtatct gccttttgtc |
| 6061 | ttcacatggc tgtctaccat ttgtctctgt gtctccaaat ctctctcctt ataaacacag |
| 6121 | cagttattgg attaggcccc actctaatcc agtatgaccc cattttaaca tgattacact |
| 6181 | tatttctaga taaggtcaca ttcacgtaca ccaagggtta ggaattgaac atatcttttt |
| 6241 | gggggacaca attcaaccca caagtgtcag tctctagctg agcctttccc ttcctgtttt |
| 6301 | tctccttttt agttgctatg ggttaggggc caaatctcca gtcatactag aattgCACAT |
| 6361 | GGACTGGATA TTTGGGAATA CTGCGGGTCT ATTCTATGAG CTTTAGTATG TAACATTTAA |
| 6421 | TATCAGTGTA AAGAAGCCCT TTTTTAAGTT ATTTCTTTGA ATTTCTAAAT GTATGCCCTG |
| 6481 | AATATAAGTA ACAAGTTACC ATGTCTTGTA AAATGAtcat atcaacaaac atttaatgtg |
| 6541 | cacctactgt gctagttgAA TGTCTTTATC CTGATAGGAG ATAACAGGAT TCCACATCTT |
| 6601 | TGACTTAAGA GGACAAACCA AATATGTCTA AATCATTTGG GGTTTTGATG GATATCTTTA |
| 6661 | AATTGCTGAA CCTAATCATT GGTTTCATAT GTCATTGTTT AGATATCTCC GGAGCATTTG |
| 6721 | GATAATGTGA CAGTTGGAAT GCAGTGATGT CGACTCTTTG CCCACCGCCA TCTCCAGCTG |
| 6781 | TTGCCAAGAC AGAGATTGCT TTAAGTGGCA AATCACCTTT ATTAGCAGCT ACTTTTGCTT |
| 6841 | ACTGGGACAA TATTCTTGGT CCTAGAGTAA GGCACATTTG GGCTCCAAAG ACAGAACAGG |
| 6901 | TACTTCTCAG TGATGGAGAA ATAACTTTTC TTGCCAACCA CACTCTAAAT GGAGAAATCC |
| 6961 | TTCGAAATGC AGAGAGTGGT GCTATAGATG TAAAGTTTTT TGTCTTGTCT GAAAAGGGAG |
| 7021 | TGATTATTGT TTCATTAATC TTTGATGGAA ACTGGAATGG GGATCGCAGC ACATATGGAC |
| 7081 | TATCAATTAT ACTTCCACAG ACAGAACTTA GTTTCTACCT CCCACTTCAT AGAGTGTGTG |
| 7141 | TTGATAGATT AACACATATA ATCCGGAAAG GAAGAATATG GATGCATAAG GTAAGTGATT |
| 7201 | TTTCAGCTTA TTAATCATGT TAACCTATCT GTTGAAAGCT TATTTTCTGG TACATATAAA |
| 7261 | TCTTATTTTT TTAATTATAT GCAGTGAACA TCAAACAATA AATGTTATTT ATTTTGCATT |
| 7321 | TACCCTATTA GATACAAATA CATCTGGTCT GATACCTGTC atcttcatat taactgtgga |
| 7381 | aggtacgaaa tggtagctcc acattataga tgaaaagcta aagcttagac aaataaagaa |
| 7441 | acttTTAGAC CCTGGATTCT TCTTGGGAGC CTTTGACTCT AATACCTTTT GTTTCCCTTT |
| 7501 | CATTGCACAA TTCTGTCTTT TGCTTACTAC TATGTGTAAG TATAACAGTT CAAAGTAATA |
| 7561 | GTTTCATAAG CTGTTGGTCA tgtagccttt ggtctcttta acctctttgc caagttccca |
| 7621 | ggttcataaa atgaggaggt tgaatggaat ggttcccaag agaattcctt ttaatcttac |
| 7681 | aGAAATTATT GTTTTCCTAA ATCCTGTAGT TGAATATATA ATGCTATTTA CATTTCAGTA |
| 7741 | TAGTTTTGAT GTATCTAAAG AACACATTGA ATTCTCCTTC CTGTGTTCCA GTTTGATACT |
| 7801 | AACCTGAAAG TCCATTAAGC ATTACCAGTT TTAAAAGGCT TTTGCCCAAT AGTAAGGAAA |
| 7861 | AATAATATCT TTTAAAAGAA TAATTTTTTA CTATGTTTGC AGGCTTACTT CCTTTTTTCT |
| 7921 | CACATTATGA AACTCTTAAA ATCAGGAGAA TCTTTTAAAC AACATCATAA TGTTTAATTT |
| 7981 | GAAAAGTGCA AGTCATTCTT TTCCTTTTTG AAACTATGCA GATGTTACAT TGACTGTTTT |
| 8041 | CTGTGAAGTT ATCTTTTTTT CACTGCAGAA TAAAGGTTGT TTTGATTTTA TTTTGTATTG |
| 8101 | TTTATGAGAA CATGCATTTG TTGGGTTAAT TTCCTACCCC TGCCCCCATT TTTTCCCTAA |
| 8161 | AGTAGAAAGT ATTTTTCTTG TGAACTAAAT TACTACACAA GAACATGTCT ATTGAAAAAT |
| 8221 | AAGCAAGTAT CAAAATGTTG TGGGTTGTTT TTTTAAATAA ATTTTCTCTT GCTCAGGAAA |
| 8281 | GACAAGAAAA TGTCCAGAAG ATTATCTTAG AAGGCACAGA GAGAATGGAA GATCAGGTAT |
| 8341 | ATGCAAATTG CATACTGTCA AATGTTTTTC TCACAGCATG TATCTGTATA AGGTTGATGG |
| 8401 | CTACATTTGT CAAGGCCTTG GAGACATACG AATAAGCCTT TAATGGAGCT TTTATGGAGG |
| 8461 | TGTACAGAAT AAACTGGAGG AAGATTTCCA TATCTTAAAC CCAAAGAGTT AAATCAGTAA |
| 8521 | ACAAAGGAAA ATAGTAATTG CATCTACAAA TTAATATTTG CTCCCttttt ttttCTGTTT |
| 8581 | GCCCAGAATA AATTTTGGAT AACTTGTTCA TAGTaaaaat aaaaaaaaTT GTCTCTGATA |
| 8641 | TGTTCTTTAA GGTACTACTT CTCGAACCTT TCCCTAGAAG TAGCTGTAAC AGAAGGAGAG |
| 8701 | CATATGTACC CCTGAGGTAT CTGTCTGGGG TGTAGGCCCA GGTCCACACA ATATTTCTTC |
| 8761 | TAAGTCTTAT GTTGTATCGT TAAGACTCAT GCAATTTACA TTTTATTCCA TAACTATTTT |
| 8821 | AGTATTAAAA TTTGTCAGTG ATATTTCTTA CCCTCTCctc taggaaaatg tgccatgttt |
| 8881 | atcccttggc tttgaatgcc cctcAGGAAC AGACACTAAG AGTTTGAGAA GCATGGTTAC |
| 8941 | AAGGGTGTGG CTTCCCCTGC GGAAACTAAG TACAGACTAT TTCACTGTAA AGCAGAGAAG |
| 9001 | TTCTTTTGAA GGAGAATCTC CAGTGAAGAA AGAGTTCTTC ACTTTTACTT CCATTTCCTC |
| 9061 | TTGTGGGTGA CCCTCAATGC TCCTTGTAAA ACTCCAATAT TTTAAACATG GCTGTTTTGC |
| 9121 | CTTTCTTTGC TTCTTTTTAG CATGAATGAG ACAGATGATA CTTTAAAAAA GTAATTaaaa |
| 9181 | aaaaaaaCTT GTGAAAATAC ATGGCCATAA TACAGAACCC AATACAATGA TCTCCTTTAC |
| 9241 | CAAATTGTTA TGTTTGTACT TTTGTAGATA GCTTTCCAAT TCAGAGACAG TTATTCTGTG |
| 9301 | TAAAGGTCTG ACTTAACAAG AAAAGATTTC CCTTTACCCA AAGAATCCCA GTCCTTATTT |
| 9361 | GCTGGTCAAT AAGCAGGGTC CCCAGGAATG GGGTAACTTT CAGCACCCTC TAACCCACTA |
| 9421 | GTTATTAGTA GACTAATTAA GTAAACTTAT CGCAAGTTGA GGAAACTTAG AACCAACTAA |
| 9481 | AATTCTGCTT TTACTGGGAT TTTGTTTTTT CAAACCAGAA ACCTTTACTT AAGTTGACTA |
| 9541 | CTATTAATGA ATTTTGGTCT CTCTTTTAAG TGCTCTTCTT AAAAATGTTA TCTTACTGCT |
| 9601 | GAGAAGTTCA AGTTTGGGAA GTACAAGGAG GAATAGAAAC TTAAGAGATT TTCTTTTAGA |
| 9661 | GCCTCTTCTG TATTTAGCCC TGTAGGAttt tttttttttt tttttttttt GGTGTTGTTG |
| 9721 | AGCTTCAGTG AGGCTATTCA TTCACTTATA CTGATAATGT CTGAGATACT GTGAATGAAA |
| 9781 | TActatgtat gcttaaacct aagaggaaat attttcccaa aattattctt cccgaaaagg |
| 9841 | aggagttgcc ttttgattga gttcttgcaa atctcacaac gactttattt tgaacaatac |
| 9901 | tgtttgggga tgatgcatta gtttgaaaca acttcagttg tagctgtcat ctgataaaat |
| 9961 | tgcttcacag ggaaggaaat ttaacacgga tctagtcatt attcttgtta gattgaatgt |
| 10021 | gtgaattgta attgtaaaca ggcatgataa ttattacttt aaaaactaaa aacagtgaat |
| 10081 | agttagttgt ggaggttact aaaggatggt ttttttttaa ataaaacttt cagcattatg |
| 10141 | caaatgggca tatggcttag gataaaactt ccagaagtag catcacattt aaattctcaa |
| 10201 | gcaacttaat aatatggggc tctgaaaaac tggttaaggt tactccaaaa atggccctgg |
| 10261 | gtctgacaaa gattctaact taaagatgct tatgaagact ttgagtaaaa tcatttcata |
| 10321 | aaataagtga ggaaaaacaa ctagtattaa attcatotta aataatgtat gatttaaaaa |
| 10381 | atatgtttag ctaaaaatgc atagtcattt gacaatttca tttatatctc aaaaaattta |
| 10441 | cttaaccaag ttggtcacaa aactgatgag actggtggtg gtagtgaata aatgagggac |
| 10501 | catccatatt tgagacactt tacatttgTG ATGTGTTATA CTGAATTTTC AGTTTGATTC |
| 10561 | TATAGACTAC AAATTTCAAA ATTACAATTT CAAGATGTAA TAAGTAGTAA TATCTTGAAA |
| 10621 | TAGCTCTAAA GGGAATTTTT CTGTTTTATT GATTCTTAAA ATATATGTGC TGATTTTGAT |
| 10681 | TTGCATTTGG GTAGATTATA CTTTTATGAG TATGGAGGTT AGGTATTGAT TCAAGTTTTC |
| 10741 | CTTACCTATT TGGTAAGGAT TTCAAAGTCT TTTTGTGCTT GGTTTTCCTC ATTTTTAAAT |
| 10801 | ATGAAATATA TTGATGACCT TTAACAAAtt ttttttATCT CAAATTTTAA AGGAGATCTT |
| 10861 | TTCTAAAAGA GGCATGATGA CTTAATCATT GCATGTAACA GTAAACGATA AACCAATGAT |
| 10921 | TCCATACTCT CTAAAGAATA AAAGTGAGCT TTAGGGCCGG GCATggtcag aaatttgaca |
| 10981 | ccaacctggc caacatggcg aaaccccgtc tctactaaaa atacaaaaat cagccgggca |
| 11041 | tggtggcggc acctatagtc ccagctactt gggaggatga gacaggagag tcacttgaac |
| 11101 | ctgggaggag aggttgcagt gagctgagat cacgccattg cactccagcc tgagcaatga |
| 11161 | aagcaaaact ccatctcaaa aaaaaaaaaa gaaaagaaag aataaaaGTG AGCTTTGGAT |
| 11221 | TGCATATAAA TCCTTTAGAC ATGTAGTAGA CTTGTTTGAT ACTGTGTTTG AACAAATTAC |
| 11281 | GAAGTATTTT CATCAAAGAA TGTTATTGTT TGATGTTATT TTTATTTTTT ATTGCCCAGC |
| 11341 | TTCTCTCATA TTACGTGATT TTCTTCACTT CATGTCACTT TATTGTGCAG GGTCAGAGTA |
| 11401 | TTATTCCAAT GCTTACTGGA GAAGTGATTC CTGTAATGGA ACTGCTTTCA TCTATGAAAT |
| 11461 | CACACAGTGT TCCTGAAGAA ATAGATGTAA GTTTAAATGA GAGCAATTAT ACACTTTATG |
| 11521 | AGTTTTTTGG GGTTATAGTA TTATTATGTA TATTATTAAT ATTCTAATTT TAATAGTAAG |
| 11581 | GACTTTGTCA TACATACTAT TCACATACAG TATTAGCCAC TTTAGCAAAT AAGCACACAC |
| 11641 | AAAATCCTGG ATTTTATGGC AAAACAGAGG CATTTTTGAT CAGTGATGAC AAAATTAAAT |
| 11701 | TCATTTTGTT TATTTCATTA CTTTTATAAT TCCTAAAAGT GGGAGGATCC CAGCTCTTAT |
| 11761 | AGGAGCAATT AATATTTAAT GTAGTGTCTT TTGAAACAAA ACTGTGTGCC AAAGTAGTAA |
| 11821 | CCATTAATGG AAGTTTACTT GTAGTCACAA ATTTAGTTTC CTTAATCATT TGTTGAGGAC |
| 11881 | GTTTTGAATC ACACACTATG AGTGTTAAGA GATACCTTTA GGAAACTATT CTTGTTGTTT |
| 11941 | TCTGATTTTG TCATTTAGGT TAGTCTCCTG ATTCTGACAG CTCAGAAGAG GAAGTTGTTC |
| 12001 | TTGTAAAAAT TGTTTAACCT GCTTGACCAG CTTTCACATT TGTTCTTCTG AAGTTTATGG |
| 12061 | TAGTGCACAG AGATTGTTTT TTGGGGAGTC TTGATTCTCG GAAATGAAGG CAGTGTGTTA |
| 12121 | TATTGAATCC AGACTTCCGA AAACTTGTAT ATTAAAAGTG TTATTTCAAC ACTATGTTAC |
| 12181 | AGCCAGACTA Atttttttat tttttGATGC ATTTTAGATA GCTGATACAG TACTCAATGA |
| 12241 | TGATGATATT GGTGACAGCT GTCATGAAGG CTTTCTTCTC AAGTAAGAAT TTTTCTTTTC |
| 12301 | ATAAAAGCTG GATGAAGCAG ATACCATCTT ATGCTCACCT ATGACAAGAT TTGGAAGAAA |
| 12361 | GAAAATAACA GACTGTCTAC TTAGATTGTT CTAGGGACAT TACGTATTTG AACTGTTGCT |
| 12421 | TAAATTTGTG TTATTTTTCA CTCATTATAT TTCTATATAT ATTTGGTGTT ATTCCATTTG |
| 12481 | CTATTTAAAG AAACCGAGTT TCCATCCCAG ACAAGAAATC ATGGCCCCTT GCTTGATTCT |
| 12541 | GGTTTCTTGT TTTACTTCTC ATTAAAGCTA ACAGAATCCT TTCATATTAA GTTGTACTGT |
| 12601 | AGATGAACTT AAGTTATTTA GGCGTAGAAC AAAATTATTC ATATTTATAC TGATCTTTTT |
| 12661 | CCATCCAGca gtggagttta gtacttaaga gtttgtgccc ttaaaccaga ctccctggat |
| 12721 | taatgctgtg tacccgtggg caaggtgcct gaattctcta tacacctatt tcctcatctg |
| 12781 | taaaatggca ataatagtaa tagtacctaa tgtgtagggt tgttataagc attgagtaag |
| 12841 | ataaataata taaagcactt agaacagtgc ctggaacata aaaacactta ataaTAGCTC |
| 12901 | ATAGCTAACA TTTCCTATTT ACATTTCTTC TAGAaatagc cagtatttgt tgagtgccta |
| 12961 | catgttagtt cctttactag ttgctttaca tgtattatct tatATTCTGT TTTAAAGTTT |
| 13021 | CTTCACAGTT ACAGATTTTC ATGAAATTTT ACTTTTAATA AAAGAGAAGT AAAAGTATAA |
| 13081 | AGTATTCACT TTTATGTTCA CAGTCTTTTC CTTTAGGCTC ATGATGGAGT ATCAGAGGCA |
| 13141 | TGAGTGTGTT TAACCTAAGA GCCTTAATGG CTTGAATCAG AAGCACTTTA GTCCTGTATC |
| 13201 | TGTTCAGTGT CAGCCTTTCA TACATCATTT TAAATCCCAT Ttgactttaa gtaagtcact |
| 13261 | taatctctct acatgtcaat ttcttcagct ataaaatgat ggtatttcaa taaataaata |
| 13321 | cattaattaa atgatattat actgactaat tgggctgttt taaggctcaa taagaaaatt |
| 13381 | tctgtgaaag gtctctagaa aatgtaggtt cctatacaaa taaaagATAA CATTGTGCTT |
| 13441 | ATAGCTTCGG TGTTTATCAT ATAAAGCTAT TCTGAGTTAT TTGAAGAGCT CACCTACttt |
| 13501 | tttttgtttt tagtttgtta aattgtttta taggcaatgt ttttaATCTG TTTTCTTTAA |
| 13561 | CTTACAGTGC CATCAGCTCA CACTTGCAAA CCTGTGGCTG TTCCGTTGTA GTAGGTAGCA |
| 13621 | GTGCAGAGAA AGTAAATAAG GTAGTTTATT TTATAATCTA GCAAATGATT TGACTCTTTA |
| 13681 | AGACTGATGA TATATCATGG ATTGTCATTT AAATGGTAGG TTGCAATTAA AATGATCTAG |
| 13741 | TAGTATAAGG AGGCAATGTA ATCTCATCAA ATTGCTAAGA CACCTTGTGG CAACAGTGAG |
| 13801 | TTTGAAATAA ACTGAGTAAG AATCATTTAT CAGTTTATTT TGATAGCTCG GAAATACCAG |
| 13861 | TGTCAGTAGT GTATAAATGG TTTTGAGAAT ATATTAAAAT CAGATATATa aaaaaaaTTA |
| 13921 | CTCTTCTATT TCCCAATGTT ATCTTTAACA AATCTGAAGA TAGTCATGTA CTTTTGGTAG |
| 13981 | TAGTTCCAAA GAAATGTTAT TTGTTTATTC ATCTTGATTT CATTGTCTTC GCTTTCCTTC |
| 14041 | TAAATCTGTC CCTTCTAGGG AGCTATTGGG ATTAAGTGGT CATTGATTAT TATACTTTAT |
| 14101 | TCAGTAATGT TTCTGACCCT TTCCTTCAGT GCTACTTGAG TTAATTAAGG ATTAATGAAC |
| 14161 | AGTTACATTT CCAAGCATTA GCTAATAAAC TAAAGGATTT TGCACTTTTC TTCACTGACC |
| 14221 | ATTAGTTAGA AAGAGTTCAG AGATAAGTAT GTGTATCTTT CAATTTCAGC AAACCTAATT |
| 14281 | TTTTAAAAAA AGTTTTACAT AGGAAATATG TTGGAAATGA TACTTTACAA AGATATTCAT |
| 14341 | AAtttttttt tGTAATCAGC TACTTTGTAT ATTTACATGA GCCTTAATTT ATATTTCTCA |
| 14401 | TATAACCATT TATGAGAGCT TAGTATACCT GTGTCATTAT ATTGCATCTA CGAACTAGTG |
| 14461 | ACCTTATTCC TTCTGTTACC TCAAACAGGT GGCTTTCCAT CTGTGATCTC CAAAGCCTTA |
| 14521 | GGTTGCACAG AGTGACTGCC GAGCTGCTTT ATGAAGGGAG AAAGGCTCCA TAGTTGGAGT |
| 14581 | Gttttttttt ttttttttAA ACATTTTTCC CATCCTCCAT CCTCTTGAGG GAGAATAGCT |
| 14641 | TACCTTTTAT CTTGTTTTAA TTTGAGAAAG AAGTTGCCAC CACTCTAGGT TGAAAACCAC |
| 14701 | TCCTTTAACA TAATAACTGT GGATATGGTT TGAATTTCAA GATAGTTACA TGCCTTTTTA |
| 14761 | TTTTTCCTAA TAGAGCTGTA GGTCAAATAT TATTAGAATC AGATTTCTAA ATCCCACCCA |
| 14821 | ATGACCTGCT TATTTTAAAT CAAATTCAAT AATTAATTCT CTTCTTTTTG GAGGATCTGG |
| 14881 | ACATTCTTTG ATATTTCTTA CAACGAATTT CATGTGTAGA CCCACTAAAC AGAAGCTATa |
| 14941 | aaagttgcat ggtcaaataa gtctgagaaa gtctgcagat gatataattc acctgaagag |
| 15001 | tcacagtatg tagccaaatg ttaaaggttt tgagatgcca tacagtaaat ttaccaagca |
| 15061 | ttttctaaat ttatttgacc acagaatccc tattttaagc aacaactgtt acatcccatg |
| 15121 | gaTTCCAGGT GACTAAAGAA TACTTATTTC TTAGGATATG TTTTATTGAT AATAACAATT |
| 15181 | AAAATTTCAG ATATCTTTCA TAAGCAAATC AGTGGTCTTT TTACTTCATG TTTTAATGCT |
| 15241 | AAAATATTTT CTTTTATAGA TAGTCAGAAC ATTATGCCTT TTTCTGACTC CAGCAGAGAG |
| 15301 | AAAATGCTCC AGGTTATGTG AAGCAGAATC ATCATTTAAA TATGAGTCAG GGCTCTTTGT |
| 15361 | ACAAGGCCTG CTAAAGGTAT AGTTTCTAGT TATCACAAGT GAAACCACTT TTCTAAAATC |
| 15421 | ATTTTTGAGA CTCTTTATAG ACAAATCTTA AATATTAGCA TTTAATGTAT CTCATATTGA |
| 15481 | CATGCCCAGA GACTGACTTC CTTTACACAG TTCTGCACAT AGACTATATG TCTTATGGAT |
| 15541 | TTATAGTTAG TATCATCAGT GAAACACCAT AGAATACCCT TTGTGTTCCA GGTGGGTCCC |
| 15601 | TGTTCCTACA TGTCTAGCCT CAGGACtttt ttttttttAA CACATGCTTA AATCAGGTTG |
| 15661 | CACATCAAAA ATAAGATCAT TTCTTTTTAA CTAAATAGAT TTGAATTTTA TTGaaaaaaa |
| 15721 | aTTTTAAACA TCTTTAAGAA GCTTATAGGA TTTAAGCAAT TCCTATGTAT GTGTACTAAA |
| 15781 | atatatatat ttctatatat aatatatatT AGAAAAAAAT TGTATTTTTC TTTTATTTGA |
| 15841 | GTCTACTGTC AAGGAGCAAA ACAGAGAAAT GTAAATTAGC AATTATTTAT AATACTTAAA |
| 15901 | GGGAAGAAAG TTGTTCACCT TGTTGAATCT ATTATTGTTA TTTCAATTAT AGTCCCAAGA |
| 15961 | CGTGAAGAAA TAGCTTTCCT AATGGTTATG TGATTGTCTC ATAGTGACTA CTTTCTTGAG |
| 16021 | GATGTAGCCA CGGCaaaatg aaataaaaaa atttaaaaat tGTTGCAAAT ACAAGTTATA |
| 16081 | TTAGGCTTTT GTGCATTTTC AATAATGTGC TGCTATGAAC TCAGAATGAT AGTATTTAAA |
| 16141 | TATAGAAACT AGTTAAAGGA AACGTAGTTT CTATTTGAGT TATACATATC TGTAAATTAG |
| 16201 | AACTTCTCCT GTTAAAGGCA TAATAAAGTG CTTAATACTT TTGTTTCCTC AGCACCCTCT |
| 16261 | CATTTAATTA TATAATTTTA GTTCTGAAAG GGACCTATAC CAGATGCCTA GAGGAAATTT |
| 16321 | CAAAACTATG ATCTAATGAA AAAATATTTA ATAGTTCTCC ATGCAAATAC AAATCATATA |
| 16381 | GTTTTCCAGA AAATACCTTT GACATtatac aaagatgatt atcacagcat tataatagta |
| 16441 | aaaaaatgga aatagcctCT TTCTTCTGTT CTGTTCAtag cacagtgcct catacgcagt |
| 16501 | aggttattat tacatggtaa ctGGCTACCC CAACTGATTA GGAAAGAAGT AAATTTGTTT |
| 16561 | TATAAAAATA CATACTCATT GAGGTGCATA GAATAATTaa gaaattaaaa gacacttgta |
| 16621 | attttgaatc cagtgaatac ccactgttaa tatttggtat atctctttct agtctttttt |
| 16681 | tcccttttgc atgtattttc tttaagactc ccacccccac tggatcatct ctgcatgttc |
| 16741 | taatctgctt ttttcacagc agattctaag cctctttgaa tatcaacaca aacttcaaca |
| 16801 | acttcatcta tagatgccaa ataataaatt catttttatt tacttaacca cttcctttgg |
| 16861 | atgcttaggt cattctgatg ttttgctatt gaaaccaatg ctatactgaa cacttctgtc |
| 16921 | actaaaactt tgcacacact catgaatagc ttcttaggat aaatttttag agatggattt |
| 16981 | gctaaatcag agACCATTTT TTAAAATTAA AAAACAATTA TTCATATCGT TTGGCATGTA |
| 17041 | AGACAGTAAA TTTTCCTTTT ATTTTGACAG GATTCAACTG GAAGCTTTGT GCTGCCTTTC |
| 17101 | CGGCAAGTCA TGTATGCTCC ATATCCCACC ACACACATAG ATGTGGATGT CAATACTGTG |
| 17161 | AAGCAGATGC CACCCTGTCA TGAACATATT TATAATCAGC GTAGATACAT GAGATCCGAG |
| 17221 | CTGACAGCCT TCTGGAGAGC CACTTCAGAA GAAGACATGG CTCAGGATAC GATCATCTAC |
| 17281 | ACTGACGAAA GCTTTACTCC TGATTTGTAC GTAATGCTCT GCCTGCTGGT ACTGTAGTCA |
| 17341 | AGCAATATGA AATTGTGTCT TTTACGAATA AAAACAAAAC AGAAGTTGCA TTTAAAAAGA |
| 17401 | AAGAAATATT ACCAGCAGAA TTATGCTTGA AGAAACATTT AATCAAGCAT TTTTTTCTTA |
| 17461 | AATGTTCTTC TTTTTCCATA CAATTGTGTT TACCCTAAAA TAGGTAAGAT TAACCCTTAA |
| 17521 | AGTAAATATT TAACTatttg tttaataaat atatattgag ctcctaggca ctgttctagg |
| 17581 | taccgggctt aatagtggcc aaccagacag ccccagcccc agcccctaca ttgtgtatag |
| 17641 | tctaTTATGT AACAGTTATT GAATGGACTT ATTAACAAAA CCAAAGAAGT AATTCTAAGT |
| 17701 | CttttttttC TTGACATATG AATATAAAAT ACAGCAAAAC TGTTAAAATA TATTAATGGA |
| 17761 | ACAttttttt actttgcatt ttatattgtt attcacttct tatttttttt taaaaaaaaa |
| 17821 | aGCCTGAACA GTAAATTCAA AAGGAAAAGT AATGATAATT AATTGTTGAG CATGGACCCA |
| 17881 | ACTTGaaaaa aaaaaTGATG ATGATAAATC TATAATCCTA AAACCCTAAG TAAACACTTA |
| 17941 | AAAGATGTTC TGAAATCAGG AAAAGAATTA TAGTATACTT TTGTGTTTCT CTTTTATCAG |
| 18001 | TTGAAAAAAg gcacagtagc tcatgcctgt aagaacagag ctttgggagt gcaaggcagg |
| 18061 | cggatcactt gaggccagga gttccagacc agcctgggca acatagtgaa accccatctc |
| 18121 | tacaaaaaat aaaaaagaat tattggaatg tgtttctgtg tgcctgtaat cctagctatt |
| 18181 | ccgaaagctg aggcaggagg atcttttgag cccaggagtt tgaggttaca gggagttatg |
| 18241 | atgtgccagt gtactccagc ctggggaaca ccgagactct gtcttattta aaaaaaaaaa |
| 18301 | aaaaaaaaTg cttgcaataa tgcctggcac atagaaggta acagtaagtg ttaactgtaa |
| 18361 | tAACCCAGGT CTAAGTGTGT AAGGCAATAG AAAAATTGGG GCAAATAAGC CTGACCTATG |
| 18421 | TATCTACAGA ATCAGTTTGA GCTTAGGTAA CAGACCTGTG GAGCACCAGT AATTACACAG |
| 18481 | TAAGTGTTAA CCAAAAGCAT AGAATAGGAA TATCTTGTTC AAGGGACCCC CAGCCTTATA |
| 18541 | CATCTCAAGG TGCAGAAAGA TGACTTAATA TAGGACCCAT TTTTTCCTAG TTCTCCAGAG |
| 18601 | TTTTTATTGG TTCTTGAGAA AGTAGTAGGG GAATGTTTTA GAAAATGAAT TGGTCCAACT |
| 18661 | GAAATTACAT GTCAGTAAGT TTTTATATAT TGGTAAATTT TAGTAGACAT GTAGAAGTTT |
| 18721 | TCTAATTAAT CTGTGCCTTG AAACAttttc ttttttccta aagtgcttag tattttttcc |
| 18781 | gttttttgAT TGGTTACTTG GGAGCTTTTT TGAGGAAATT TAGTGAACTG CAGAATGGGT |
| 18841 | TTGCAACCAT TTGGTAtttt tgttttgttt tttAGAGGAT GTATGTGTAT TTTAACATTT |
| 18901 | CTTAATCATT TTTAGCCAGC TATGTTTGTT TTGCTGATTT GACAAACTAC AGTTAGACAG |
| 18961 | CTATTCTCAT TTTGCTGATC ATGACAAAAT AATATCCTGA ATTTTTAAAT TTTGCATCCA |
| 19021 | GCTCTAAATT TTCTAAACAT AAAATTGTCC AAAAAATAGT ATTTTCAGCC ACTAGATTGT |
| 19081 | GTGTTAAGTC TATTGTCACA GAGTCAtttt acttttaagt atatgttttt acatgttaat |
| 19141 | tatgtttgtt atttttaatt ttaaCTTTTT AAAATAATTC CAGTCACTGC CAATACATGA |
| 19201 | AAAATTGGTC ACTGGAAttt tttttttgac ttttatttta ggttcatgtg tacatgtgca |
| 19261 | ggtgtgttat acaggtaaat tgcgtgtcat gagggtttgg tgtacaggtg atttcattac |
| 19321 | ccaggtaata agcatagtac ccaataggta gttttttgat cctcaccctt ctcccaccct |
| 19381 | caagtaggcc ctggtgttgc tgtttccttc tttgtgtcca tgtatactca gtgtttagct |
| 19441 | cccacttaga agtgagaaca tgcggtagtt ggttttctgt tcctggatta gttcacttag |
| 19501 | gataatgacc tctagctcca tctggttttt atggctgcat agtattccat ggtgtatatg |
| 19561 | tatcacattt tctttatcca gtctaccatt gataggcatt taggttgatt ccctgtcttt |
| 19621 | gttatcatga atagtgctgt gatgaacata cacatgcatg tgtctttatg gtagaaaaat |
| 19681 | ttgtattcct ttaggtacat atagaataat ggggttgcta gggtgaatgg tagttctatt |
| 19741 | ttcagttatt tgagaaatct tcaaactgct tttcataata gctaaactaa tttacagtcc |
| 19801 | cgccagcagt gtataagtgt tcccttttct ccacaacctt gccaacatct gtgatttttt |
| 19861 | gactttttaa taatagccat tcctagagaa ttgatttgca attctctatt agtgatatta |
| 19921 | agcatttttt catatgcttt ttagctgtct gtatatattc ttctgaaaaa ttttcatgtc |
| 19981 | ctttgcccag tttgtagtgg ggtgggttgt tttttgcttg ttaattagtt ttaagttcct |
| 20041 | tccagattct gcatatccct ttgttggata catggtttgc agatattttt ctcccattgt |
| 20101 | gtaggttgtc ttttactctg ttgatagttt cttttgccat gcaggagctc gttaggtccc |
| 20161 | atttgtgttt gtttttgttg cagttgcttt tggcgtcttc atcataaaat ctgtgccagg |
| 20221 | gcctatgtcc agaatggtat ttcctaggtt gtcttccagg gtttttacaa ttttagattt |
| 20281 | tacgtttatg tctttaatcc atcttgagtt gatttttgta tatggcacaa ggaaggggtc |
| 20341 | cagtttcact ccaattccta tggctagcaa ttatcccagc accatttatt gaatacggag |
| 20401 | tcctttcccc attgcttgtt ttttgtcaac tttgttgaag atcagatggt tgtaagtgtg |
| 20461 | tggctttatt tcttggctct ctattctcca ttggtctatg tgtctgtttt tataacagta |
| 20521 | ccctgctgtt caggttccta tagcctttta gtataaaatc ggctaatgtg atgcctccag |
| 20581 | ctttgttctt tttgcttagg attgctttgg ctatttgggc tcctttttgg gtccatatta |
| 20641 | attttaaaac agttttttct ggttttgtga aggatatcat tggtagttta taggaatagc |
| 20701 | attgaatctg tagattgctt tgggcagtat ggccatttta acaatattaa ttcttcctat |
| 20761 | ctatgaatat ggaatgtttt tccatgtgtt tgtgtcatct ctttatacct gatgtataaa |
| 20821 | gaaaagctgg tattattcct actcaatctg ttccaaaaaa ttgaggagga ggaactcttc |
| 20881 | cctaatgagg ccagcatcat tctgatacca aaacctggca gagacacaac agaaaaaaga |
| 20941 | aaacttcagg ccaatatcct tgatgaatat agatgcaaaa atcctcaaca aaatactagc |
| 21001 | aaaccaaatc cagcagcaca tcaaaaagct gatctacttt gatcaagtag gctttatccc |
| 21061 | tgggatgcaa ggttggttca acatacacaa atcaataagt gtgattcatc acataaacag |
| 21121 | agctaaaaac aaaaaccaca agattatctc aataggtaga gaaaaggttg tcaataaaat |
| 21181 | ttaacatcct ccatgttaaa aaccttcagt aggtcaggtg tagtgactca cacctgtaat |
| 21241 | cccagcactt tgggaggcca aggcgggcat atctcttaag cccaggagtt caagacgagc |
| 21301 | ctaggcagca tggtgaaacc ccatctctac aaaaaaaaaa aaaaaaaaaa attagcttgg |
| 21361 | tatggtgaca tgcacctata gtcccagcta ttcaggaggt tgaggtggga ggattgtttg |
| 21421 | agcccgggag gcagaggttg gcagcgagct gagatcatgc caccgcactc cagcctgggc |
| 21481 | aacggagtga gaccctgtct caaaaaagaa aaatcacaaa caatcctaaa caaactaggc |
| 21541 | attgaaggaa catgcctcaa aaaaataaga accatctatg acagacccat agccaatatc |
| 21601 | ttaccaaatg ggcaaaagct ggaagtattc tccttgagaa ccgtaacaag acaaggatgt |
| 21661 | ccactctcac cactcctttt cagcatagtt ctggaagtcc tagccagagc aatcaggaaa |
| 21721 | gagaaagaaa gaaagacatt cagataggaa gagaagaagt caaactattt ctgtttgcag |
| 21781 | gcagtataat tctgtaccta gaaaatctca tagtctctgc ccagaaactc ctaaatctgt |
| 21841 | taaaaatttc agcaaagttt tggcattctc tatactccaa caccttccaa agtgagagca |
| 21901 | aaatcaagaa cacagtccca ttcacaatag ccgcaaaacg aataaaatac ctaggaatcc |
| 21961 | agctaaccag ggaggtgaaa gatctctatg agaattacaa aacactgctg aaagaaatca |
| 22021 | gagatgacac aaacaaatgg aaaTGTTCTT TTTTAACACC TTGCTTTATC TAATTCACTT |
| 22081 | ATGATGAAGA TACTCATTCA GTGGAACAGG TATAATAAGT CCACTCGATT AAATATAAGC |
| 22141 | CTTATTCTCT TTCCAGAGCC CAAGAAGGGG CACTATCAGT GCCCAGTCAA TAATGACGAA |
| 22201 | ATGCTAATAT TTTTCCCCTT TACGGTTTCT TTCTTCTGTA GTGTGGTACA CTCGTTTCTT |
| 22261 | AAGATAAGGA AACTTGAACT ACCTTCCTGT TTGCTTCTAC ACATACCCAT TCTCTTTTTT |
| 22321 | TGCCACTCTG GTCAGGTATA GGATGATCCC TACCACTTTC AGTTAAAAAC TCCTCCTCTT |
| 22381 | ACTAAATGTT CTCTTACCCT CTGGCCTGAG TAGAACCTAG GGAAAATGGA AGAGAAAAAG |
| 22441 | ATGAAAGGGA GGTGGGGCCT GGGAAGGGAA TAAGTAGTCC TGTTTGTTTG TGTGTTTGCT |
| 22501 | TTAGCACCTG CTATATCCTA GGTGCTGTGT TAGGCACACA TTATTTTAAG TGGCCATTAT |
| 22561 | ATTACTACTA CTCACTCTGG TCGTTGCCAA GGTAGGTAGT ACTTTCTTGG ATAGTTGGTT |
| 22621 | CATGTTACTT ACAGATGGTG GGCTTGTTGA GGCAAACCCA GTGGATAATC ATCGGAGTGT |
| 22681 | GTTCTCTAAT CTCACTCAAA tttttcttca cattttttgg tttgttttgg tttttgatgg |
| 22741 | tagtggctta tttttgttgc tggtttgttt tttgtttttt tttgAGATGG CAAGAATTGG |
| 22801 | TAGTTTTATT TATTAATTGC CTAAGGGTCT CTACTTTTTT TAAAAGATGA GAGTAGTAAA |
| 22861 | ATAGATTGAT AGATACATAC ATACCCTTAC TGGGGACTGC TTATATTCTT TAGAGAAAAA |
| 22921 | ATTACATATT AGCCTGACAA ACACCAGTAA AATGTAAATA TATCCTTGAG TAAATAAATG |
| 22981 | AATGTATATT TTGTGTCTCC AAATATATAT ATCTATATTC TTACAAATGT GTTTATATGT |
| 23041 | AATATCAATT TATAAGAACT TAAAATGTTG GCTCAAGTGA GGGATTGTGG AAGGTAGCAT |
| 23101 | TATATGGCCA TTTCAACATT TGAActtttt tcttttcttc attttcttct tttcttcAGG |
| 23161 | AATATTTTTC AAGATGTCTT ACACAGAGAC ACTCTAGTGA AAGCCTTCCT GGATCAGGTA |
| 23221 | AATGTTGAAC TTGAGATTGT CAGAGTGAAT GATATGACAT GTTTTCTTTT TTAATATATC |
| 23281 | CTACAATGCC TGTTCTATAT ATTTATATTC CCCTGGATCA TGCCCCAGAG TTCTGCTCAG |
| 23341 | CAATTGCAGT TAAGTTAGTT ACACTACAGT TCTCAGAAGA GTCTGTGAGG GCATGTCAAG |
| 23401 | TGCATCATTA CATTGGTTGC CTCTTGTCCT AGATTTATGC TTCGGGAATT CAGACCTTTG |
| 23461 | TTTACAATAT AATAAATATT ATTGCTATCT TTTAAAGATA TAATAATAAG ATATAAAGTT |
| 23521 | GACCACAACT ACTGTTTTTT GAAACATAGA ATTCCTGGTT TACATGTATC AAAGTGAAAT |
| 23581 | CTGACTTAGC TTTTACAGat ataatatata catatatata tCCTGCAATG CTTGTACTAT |
| 23641 | ATATGTAGTA CAAGtatata tatatgtttg tgtgtgtata tatatatagt acgagcatat |
| 23701 | atacatatta ccagcattgt aggatatata tatgtttata tattaaaaaa aaGTTATAAA |
| 23761 | CTTAAAACCC TATtatgtta tgtagagtat atgttatata tgatatgtaa aatatataac |
| 23821 | atatactcta tgatagagtg taatatattt tttatatata ttttaacATT TATAAAATGA |
| 23881 | TAGAATTAAG AATTGAGTCC TAATCTGTTT TATTAGGTGC TTTTTGTAGT GTCTGGTCTT |
| 23941 | TCTAAAGTGT CTAAATGATT TTTCCTTTTG ACTTATTAAT GGGGAAGAGC CTGTATATTA |
| 24001 | ACAATTAAGA GTGCAGCATT CCATACGTCA AACAACAAAC ATTTTAATTC AAGCATTAAC |
| 24061 | CTATAACAAG TAAGtttttt tttttttttt GAGAAAGGGA GGTTGTTTAT TTGCCTGAAA |
| 24121 | TGACTCAAAA ATATTTTTGA AACATAGTGT ACTTATTTAA ATAACATCTT TATTGTTTCA |
| 24181 | TTCTTTTAAA AAATATCTAC TTAATTACAC AGTTGAAGGA AATCGTAGAT TATATGGAAC |
| 24241 | TTATTTCTTA ATATATTACA GTTTGTTATA ATAACATTCT GGGGATCAGG CCAGGAAACT |
| 24301 | GTGTCATAGA TAAAGCTTTG AAATAATGAG ATCCTTATGT TTACTAGAAA TTTTGGATTG |
| 24361 | AGATCTATGA GGTCTGTGAC ATATTGCGAA GTTCAAGGAA AATTCGTAGG CCTGGAATTT |
| 24421 | CATGCTTCTC AAGCTGACAT AAAATCCCTC CCACTCTCCA CCTCATCATA TGCACACATT |
| 24481 | CTACTCCTAC CCACCCACTC CACCCCCTGC AAAAGTACAG GTATATGAAT GTCTCAAAAC |
| 24541 | CATAggctca tcttctagga gcttcaatgt tatttgaaga tttgggcaga aaaaattaag |
| 24601 | taatacgaaa taacttatgt atgagtttta aaagtgaagt aaacatggat gtattctgaa |
| 24661 | gtagaatgca aaatttgaat gcatttttaa agataaatta gaaaacttct aaaaaCTGTC |
| 24721 | AGATTGTctg ggcctggtgg cttatgcctg taatcccagc actttgggag tccgaggtgg |
| 24781 | gtggatcaca aggtcaggag atcgagacca tcctgccaac atggtgaaac cccgtctcta |
| 24841 | ctaagtatac aaaaattagc tgggcgtggc agcgtgtgcc tgtaatccca gctacctggg |
| 24901 | aggctgaggc aggagaatcg cttgaaccca ggaggtgtag gttgcagtga gtcaagatcg |
| 24961 | cgccactgca ctttagcctg gtgacagagc tagactccgt ctcaaaaaaa aaaaaaaaTA |
| 25021 | TCAGATTGTT CCTACACCTA GTGCTTCTAT ACCACACTCC TGTTAGGGGG CATCAGTGGA |
| 25081 | AATGGTTAAG GAGATGTTTA GTGTGTATTG TCTGCCAAGC ACTGTCAACA CTGTCATAGA |
| 25141 | AACTTCTGTA CGAGTAGAAT GTGAGCAAAT TATGTGTTGA AATGGTTCCT CTCCCTGCAG |
| 25201 | GTCTTTCAGC TGAAACCTGG CTTATCTCTC AGAAGTACTT TCCTTGCACA GTTTCTACTT |
| 25261 | GTCCTTCACA GAAAAGCCTT GACACTAATA AAATATATAG AAGACGATAC GTGAGTAAAA |
| 25321 | CTCCTACACG GAAGAAAAAC CTTTGTACAt tgtttttttg ttttgtttcc tttgtacatt |
| 25381 | ttctatatca taatttttgc gcttcttttt tttttttttt tttttttttt tCCATTATTT |
| 25441 | TTAGGCAGAA GGGAAAAAAG CCCTTTAAAT CTCTTCGGAA CCTGAAGATA GACCTTGATT |
| 25501 | TAACAGCAGA GGGCGATCTT AACATAATAA TGGCTCTGGC TGAGAAAATT AAACCAGGCC |
| 25561 | TACACTCTTT TATCTTTGGA AGACCTTTCT ACACTAGTGT GCAAGAACGA GATGTTCTAA |
| 25621 | TGACTTTTTA AATGTGTAAC TTAATAAGCC TATTCCATCA CAATCATGAT CGCTGGTAAA |
| 25681 | GTAGCTCAGT GGTGTGGGGA AACGTTCCCC TGGATCATAC TCCAGAATTC TGCTCTCAGC |
| 25741 | AATTGCAGTT AAGTAAGTTA CACTACAGTT CTCACAAGAG CCTGTGAGGG GATGTCAGGT |
| 25801 | GCATCATTAC ATTGGGTGTC TCTTTTCCTA GATTTATGCT TTTGGGATAC AGACCTATGT |
| 25861 | TTACAATATA ATAAATATTA TTGCTATCTT TTAAAGATAT AATAATAGGA TGTAAACTTG |
| 25921 | ACCACAACTA CTGTTTTTTT GAAATACATG ATTCATGGTT TACATGTGTC AAGGTGAAAT |
| 25981 | CTGAGTTGGC TTTTACAGAT AGTTGACTTT CTATCTTTTG GCATTCTTTG GTGTGTAGAA |
| 26041 | TTACTGTAAT ACTTCTGCAA TCAACTGAAA ACTAGAGCCT TTAAATGATT TCAATTCCAC |
| 26101 | AGAAAGAAAG TGAGCTTGAA CATAGGATGA GCTTTAGAAA GAAAATTGAT CAAGCAGATG |
| 26161 | TTTAATTGGA ATTGATTATT AGATCCTACT TTGTGGATTT AGTCCCTGGG ATTCAGTCTG |
| 26221 | TAGAAATGTC TAATAGTTCT CTATAGTCCT TGTTCCTGGT GAACCACAGT TAGGGTGTTT |
| 26281 | TGTTTATTTT ATTGTTCTTG CTATTGTTGA TATTCTATGT AGTTGAGCTC TGTAAAAGGA |
| 26341 | AATTGTATTT TATGTTTTAG TAATTGTTGC CAACTTTTTA AATTAATTTT CATTATTTTT |
| 26401 | GAGCCAAATT GAAATGTGCA CCTCCTGTGC CTTTTTTCTC CTTAGAAAAT CTAATTACTT |
| 26461 | GGAACAAGTT CAGATTTCAC TGGTCAGTCA TTTTCATCTT GTTTTCTTCT TGCTAAGTCT |
| 26521 | TACCATGTAC CTGCTTTGGC AATCATTGCA ACTCTGAGAT TATAAAATGC CTTAGAGAAT |
| 26581 | ATACTAACTA ATAAGATCTT TTTTTCAGAA ACAGAAAATA GTTCCTTGAG TACTTCCTTC |
| 26641 | TTGCATTTCT GCCTATGTTT TTGAAGTTGT TGCTGTTTGC CTGCAATAGG CTATAAGGAA |
| 26701 | TAGCAGGAGA AATTTTACTG AAGTGCTGTT TTCCTAGGTG CTACTTTGGC AGAGCTAAGT |
| 26761 | TATCTTTTGT TTTCTTAATG CGTTTGGACC ATTTTGCTGG CTATAAAATA ACTGATTAAT |
| 26821 | ATAATTCTAA CACAATGTTG ACATTGTAGT TACACAAACA CAAATAAATA TTTTATTTAA |
| 26881 | AATTCTGGAA GTAATATAAA AGGGAAAATA TATTTATAAG AAAGGGATAA AGGTAATAGA |
| 26941 | GCCCTTCTGC CCCCCACCCA CCAAATTTAC ACAACAAAAT GACATGTTCG AATGTGAAAG |
| 27001 | GTCATAATAG CTTTCCCATC ATGAATCAGA AAGATGTGGA CAGCTTGATG TTTTAGACAA |
| 27061 | CCACTGAACT AGATGACTGT TGTACTGTAG CTCAGTCATT TAAAAAATAT ATAAATACTA |
| 27121 | CCTTGTAGTG TCCCATACTG TGTTTTTTAC ATGGTAGATT CTTATTTAAG TGCTAACTGG |
| 27181 | TTATTTTCTT TGGCTGGTTT ATTGTACTGT TATACAGAAT GTAAGTTGTA CAGTGAAATA |
| 27241 | AGTTATTAAA GCATGTGTAA ACATTGTTAT ATATCTTTTC TCCTAAATGG AGAATTTTGA |
| 27301 | ATAAAATATA TTTGAAATTT TG |
| S92RNL (Sense GR-Nanoluciferase reporter plasmid) sequence | |
| SEQ ID NO: 57 | |
| 1 | TAGTAATCAA TTACGGGGTC ATTAGTTCAT AGCCCATATA TGGAGTTCCG CGTTACATAA |
| 61 | CTTACGGTAA ATGGCCCGCC TGGCTGACCG CCCAACGACC CCCGCCCATT GACGTCAATA |
| 121 | ATGACGTATG TTCCCATAGT AACGCCAATA GGGACTTTCC ATTGACGTCA ATGGGTGGAG |
| 181 | TATTTACGGT AAACTGCCCA CTTGGCAGTA CATCAAGTGT ATCATATGCC AAGTCCGCCC |
| 241 | CCTATTGACG TCAATGACGG TAAATGGCCC GCCTGGCATT ATGCCCAGTA CATGACCTTA |
| 301 | CGGGACTTTC CTACTTGGCA GTACATCTAC GTATTAGTCA TCGCTATTAC CATGGTGATG |
| 361 | CGGTTTTGGC AGTACACCAA TGGGCGTGGA TAGCGGTTTG ACTCACGGGG ATTTCCAAGT |
| 421 | CTCCACCCCA TTGACGTCAA TGGGAGTTTG TTTTGGCACC AAAATCAACG GGACTTTCCA |
| 481 | AAATGTCGTA ATAACCCCGC CCCGTTGACG CAAATGGGCG GTAGGCGTGT ACGGTGGGAG |
| 541 | GTCTATATAA GCAGAGCTCG TTTAGTGAAC CGTCAGATCA CTAGAAGCTT TATTGCGGTA |
| 601 | GTTTATCACA GTTAAATTGC TAACGCAGTC AGTGCTTCTG ACACAACAGT CTCGAACTTA |
| 661 | AGCTGCAGAA GTTGGTCGTG AGGCACTGGG CAGGTAAGTA TCAAGGTTAC AAGACAGGTT |
| 721 | TAAGGAGACC AATAGAAACT GGGCTTGTCG AGACAGAGAA GACTCTTGCG TTTCTGATAG |
| 781 | GCACCTATTG GTCTTACTGA CATCCACTTT GCCTTTCTCT CCACAGGTGT CCACTCCCAG |
| 841 | TTCAATTACA GCTCTTAAGG CTAGAGTACT TAATACGACT CACTATAGGG ATATCTGCTT |
| 901 | ATCGATACCG TCGACCTCGA ATCACTAGTC AGCTGGAATT CCTCACAGTA CTCGCTGAGG |
| 961 | GTGAACAAGA AAAGACCTGA TAAAGATTAA CCAGAAGAAA ACAAGGAGGG AAACAACCGC |
| 1021 | AGCCTGTAGC AAGCTCTGGA ACTCAGGAGT CGCGCGCTAG CTCTTCAGGC CGGGGCCGGG |
| 1081 | GCCGGGGCCG GGGCCGGGGC CGGGGCCGGG GCCGGGGCCG GGGCCGGGGC CGGGGCCGGG |
| 1141 | GCCGGGGCCG GGGCCGGGGC CGGGGCCGGG GCCGGGGCCG GGGCCGGGGC CGGGGCCGGG |
| 1201 | GCCGGGGCCG GGGCCGGGGC CGGGGCCGGG GCCGGGGCCG GGGCCGGGGC CGGGGCCGGG |
| 1261 | GCCGGGGCCG GGGCCGGGGC CGGGGCCGGG GCCGGGGCCG GGGCCGGGGC CGGGGCCGGG |
| 1321 | GCCGGGGCCG GGGCCGGGGC CGGGGCCGGG GCCGGGGCCG GGGCCGGGGC CGGGGCCGGG |
| 1381 | GCCGGGGCCG GGGCCGGGGC CGGGGCCGGG GCCGGGGCCG GGGCCGGGGC CGGGGCCGGG |
| 1441 | GCCGGGGCCG GGGCCGGGGC CGGGGCCGGG GCCGGGGCCG GGGCCGGGGC CGGGGCCGGG |
| 1501 | GCCGGGGCCG GGGCCGGGGC CGGGGCCGGG GCCGGGGCCG GGGCCGGGGC CGGGGCCGGG |
| 1561 | GCCGGGGCCG GGGCCGGGGC CGGGGCCGGG GCCGGGGCCG GGGCCGGGGC CGGGGCCGGG |
| 1621 | GCCTGCGGCC GCTGGTCTTC ACACTCGAAG ATTTCGTTGG GGACTGGCGA CAGACAGCCG |
| 1681 | GCTACAACCT GGACCAAGTC CTTGAACAGG GAGGTGTGTC CAGTTTGTTT CAGAATCTCG |
| 1741 | GGGTGTCCGT AACTCCGATC CAAAGGATTG TCCTGAGCGG TGAAAATGGG CTGAAGATCG |
| 1801 | ACATCCATGT CATCATCCCG TATGAAGGTC TGAGCGGCGA CCAAATGGGC CAGATCGAAA |
| 1861 | AAATTTTTAA GGTGGTGTAC CCTGTGGATG ATCATCACTT TAAGGTGATC CTGCACTATG |
| 1921 | GCACACTGGT AATCGACGGG GTTACGCCGA ACATGATCGA CTATTTCGGA CGGCCGTATG |
| 1981 | AAGGCATCGC CGTGTTCGAC GGCAAAAAGA TCACTGTAAC AGGGACCCTG TGGAACGGCA |
| 2041 | ACAAAATTAT CGACGAGCGC CTGATCAACC CCGACGGCTC CCTGCTGTTC CGAGTAACCA |
| 2101 | TCAACGGAGT GACCGGCTGG CGGCTGTGCG AACGCATTCT GGCGTAATTC TAGAGTCGGG |
| 2161 | GCGGCCGGCC GCTTCGAGCA GACATGATAA GATACATTGA TGAGTTTGGA CAAACCACAA |
| 2221 | CTAGAATGCA GTGAAAAAAA TGCTTTATTT GTGAAATTTG TGATGCTATT GCTTTATTTG |
| 2281 | TAACCATTAT AAGCTGCAAT AAACAAGTTA ACAACAACAA TTGCATTCAT TTTATGTTTC |
| 2341 | AGGTTCAGGG GGAGGTGTGG GAGGTTTTTT AAAGCAAGTA AAACCTCTAC AAATGTGGTA |
| 2401 | AAATCGATAA GGATCTGAAC GATGGAGCGG AGAATGGGCG GAACTGGGCG GAGTTAGGGG |
| 2461 | CGGGATGGGC GGAGTTAGGG GCGGGACTAT GGTTGCTGAC TAATTGAGAT GCATGCTTTG |
| 2521 | CATACTTCTG CCTGCTGGGG AGCCTGGGGA CTTTCCACAC CTGGTTGCTG ACTAATTGAG |
| 2581 | ATGCATGCTT TGCATACTTC TGCCTGCTGG GGAGCCTGGG GACTTTCCAC ACCCTAACTG |
| 2641 | ACACACATTC CACAGCGGAT CCGTCGACCG ATGCCCTTGA GAGCCTTCAA CCCAGTCAGC |
| 2701 | TCCTTCCGGT GGGCGCGGGG CATGACTATC GTCGCCGCAC TTATGACTGT CTTCTTTATC |
| 2761 | ATGCAACTCG TAGGACAGGT GCCGGCAGCG CTGTTCCGCT TCCTCGCTCA CTGACTCGCT |
| 2821 | GCGCTCGGTC GTTCGGCTGC GGCGAGCGGT ATCAGCTCAC TCAAAGGCGG TAATACGGTT |
| 2881 | ATCCACAGAA TCAGGGGATA ACGCAGGAAA GAACATGTGA GCAAAAGGCC AGCAAAAGGC |
| 2941 | CAGGAACCGT AAAAAGGCCG CGTTGCTGGC GTTTTTCCAT AGGCTCCGCC CCCCTGACGA |
| 3001 | GCATCACAAA AATCGACGCT CAAGTCAGAG GTGGCGAAAC CCGACAGGAC TATAAAGATA |
| 3061 | CCAGGCGTTT CCCCCTGGAA GCTCCCTCGT GCGCTCTCCT GTTCCGACCC TGCCGCTTAC |
| 3121 | CGGATACCTG TCCGCCTTTC TCCCTTCGGG AAGCGTGGCG CTTTCTCATA GCTCACGCTG |
| 3181 | TAGGTATCTC AGTTCGGTGT AGGTCGTTCG CTCCAAGCTG GGCTGTGTGC ACGAACCCCC |
| 3241 | CGTTCAGCCC GACCGCTGCG CCTTATCCGG TAACTATCGT CTTGAGTCCA ACCCGGTAAG |
| 3301 | ACACGACTTA TCGCCACTGG CAGCAGCCAC TGGTAACAGG ATTAGCAGAG CGAGGTATGT |
| 3361 | AGGCGGTGCT ACAGAGTTCT TGAAGTGGTG GCCTAACTAC GGCTACACTA GAAGAACAGT |
| 3421 | ATTTGGTATC TGCGCTCTGC TGAAGCCAGT TACCTTCGGA AAAAGAGTTG GTAGCTCTTG |
| 3481 | ATCCGGCAAA CAAACCACCG CTGGTAGCGG TGGTTTTTTT GTTTGCAAGC AGCAGATTAC |
| 3541 | GCGCAGAAAA AAAGGATCTC AAGAAGATCC TTTGATCTTT TCTACGGGGT CTGACGCTCA |
| 3601 | GTGGAACGAA AACTCACGTT AAGGGATTTT GGTCATGAGA TTATCAAAAA GGATCTTCAC |
| 3661 | CTAGATCCTT TTAAATTAAA AATGAAGTTT TAAATCAATC TAAAGTATAT ATGAGTAAAC |
| 3721 | TTGGTCTGAC AGTTACCAAT GCTTAATCAG TGAGGCACCT ATCTCAGCGA TCTGTCTATT |
| 3781 | TCGTTCATCC ATAGTTGCCT GACTCCCCGT CGTGTAGATA ACTACGATAC GGGAGGGCTT |
| 3841 | ACCATCTGGC CCCAGTGCTG CAATGATACC GCGAGACCCA CGCTCACCGG CTCCAGATTT |
| 3901 | ATCAGCAATA AACCAGCCAG CCGGAAGGGC CGAGCGCAGA AGTGGTCCTG CAACTTTATC |
| 3961 | CGCCTCCATC CAGTCTATTA ATTGTTGCCG GGAAGCTAGA GTAAGTAGTT CGCCAGTTAA |
| 4021 | TAGTTTGCGC AACGTTGTTG CCATTGCTAC AGGCATCGTG GTGTCACGCT CGTCGTTTGG |
| 4081 | TATGGCTTCA TTCAGCTCCG GTTCCCAACG ATCAAGGCGA GTTACATGAT CCCCCATGTT |
| 4141 | GTGCAAAAAA GCGGTTAGCT CCTTCGGTCC TCCGATCGTT GTCAGAAGTA AGTTGGCCGC |
| 4201 | AGTGTTATCA CTCATGGTTA TGGCAGCACT GCATAATTCT CTTACTGTCA TGCCATCCGT |
| 4261 | AAGATGCTTT TCTGTGACTG GTGAGTACTC AACCAAGTCA TTCTGAGAAT AGTGTATGCG |
| 4321 | GCGACCGAGT TGCTCTTGCC CGGCGTCAAT ACGGGATAAT ACCGCGCCAC ATAGCAGAAC |
| 4381 | TTTAAAAGTG CTCATCATTG GAAAACGTTC TTCGGGGCGA AAACTCTCAA GGATCTTACC |
| 4441 | GCTGTTGAGA TCCAGTTCGA TGTAACCCAC TCGTGCACCC AACTGATCTT CAGCATCTTT |
| 4501 | TACTTTCACC AGCGTTTCTG GGTGAGCAAA AACAGGAAGG CAAAATGCCG CAAAAAAGGG |
| 4561 | AATAAGGGCG ACACGGAAAT GTTGAATACT CATACTCTTC CTTTTTCAAT ATTATTGAAG |
| 4621 | CATTTATCAG GGTTATTGTC TCATGAGCGG ATACATATTT GAATGTATTT AGAAAAATAA |
| 4681 | ACAAATAGGG GTTCCGCGCA CATTTCCCCG AAAAGTGCCA CCTGACGCGC CCTGTAGCGG |
| 4741 | CGCATTAAGC GCGGCGGGTG TGGTGGTTAC GCGCAGCGTG ACCGCTACAC TTGCCAGCGC |
| 4801 | CCTAGCGCCC GCTCCTTTCG CTTTCTTCCC TTCCTTTCTC GCCACGTTCG CCGGCTTTCC |
| 4861 | CCGTCAAGCT CTAAATCGGG GGCTCCCTTT AGGGTTCCGA TTTAGTGCTT TACGGCACCT |
| 4921 | CGACCCCAAA AAACTTGATT AGGGTGATGG TTCACGTAGT GGGCCATCGC CCTGATAGAC |
| 4981 | GGTTTTTCGC CCTTTGACGT TGGAGTCCAC GTTCTTTAAT AGTGGACTCT TGTTCCAAAC |
| 5041 | TGGAACAACA CTCAACCCTA TCTCGGTCTA TTCTTTTGAT TTATAAGGGA TTTTGCCGAT |
| 5101 | TTCGGCCTAT TGGTTAAAAA ATGAGCTGAT TTAACAAAAA TTTAACGCGA ATTTTAACAA |
| 5161 | AATATTAACG CTTACAATTT GCCATTCGCC ATTCAGGCTG CGCAACTGTT GGGAAGGGCG |
| 5221 | ATCGGTGCGG GCCTCTTCGC TATTACGCCA GCCCAAGCTA CCATGATAAG TAAGTAATAT |
| 5281 | TAAGGTACGG GAGGTACTGG CCGCAATAAA ATATCTTTAT TTTCATTACA TCTGTGTGTT |
| 5341 | GGTTTTTTGT GTGAATCGAT AGTACTAACA TACGCTCTCC ATCAAAACAA AACGAAACAA |
| 5401 | AACAAACTAG CAAAATAGGC TGTCCCCAGT GCAAGTGCAG GTGCCAGAAC ATTTCTCTAT |
| 5461 | CGATAGGTAC CGAGCTCTTA CGCGTGCTAG CCCGGGCTCG AG |
| AS55RNL Nanoluciferase reporter plasmid | |
| SEQ ID NO: 58 | |
| 1 | TAGTAATCAA TTACGGGGTC ATTAGTTCAT AGCCCATATA TGGAGTTCCG CGTTACATAA |
| 61 | CTTACGGTAA ATGGCCCGCC TGGCTGACCG CCCAACGACC CCCGCCCATT GACGTCAATA |
| 121 | ATGACGTATG TTCCCATAGT AACGCCAATA GGGACTTTCC ATTGACGTCA ATGGGTGGAG |
| 181 | TATTTACGGT AAACTGCCCA CTTGGCAGTA CATCAAGTGT ATCATATGCC AAGTCCGCCC |
| 241 | CCTATTGACG TCAATGACGG TAAATGGCCC GCCTGGCATT ATGCCCAGTA CATGACCTTA |
| 301 | CGGGACTTTC CTACTTGGCA GTACATCTAC GTATTAGTCA TCGCTATTAC CATGGTGATG |
| 361 | CGGTTTTGGC AGTACACCAA TGGGCGTGGA TAGCGGTTTG ACTCACGGGG ATTTCCAAGT |
| 421 | CTCCACCCCA TTGACGTCAA TGGGAGTTTG TTTTGGCACC AAAATCAACG GGACTTTCCA |
| 481 | AAATGTCGTA ATAACCCCGC CCCGTTGACG CAAATGGGCG GTAGGCGTGT ACGGTGGGAG |
| 541 | GTCTATATAA GCAGAGCTCG TTTAGTGAAC CGTCAGATCA CTAGAAGCTT TATTGCGGTA |
| 601 | GTTTATCACA GTTAAATTGC TAACGCAGTC AGTGCTTCTG ACACAACAGT CTCGAACTTA |
| 661 | AGCTGCAGAA GTTGGTCGTG AGGCACTGGG CAGGTAAGTA TCAAGGTTAC AAGACAGGTT |
| 721 | TAAGGAGACC AATAGAAACT GGGCTTGTCG AGACAGAGAA GACTCTTGCG TTTCTGATAG |
| 781 | GCACCTATTG GTCTTACTGA CATCCACTTT GCCTTTCTCT CCACAGGTGT CCACTCCCAG |
| 841 | TTCAATTACA GCTCTTAAGG CTAGAGTACT TAATACGACT CACTATAGGG ATATCGAATT |
| 901 | CAATAGTCAC TTCCTTTAAG CAAGTCTGTG TCATCTCGGA GCTGTGAAGC AACCAGGTCA |
| 961 | TGTCCCACAG AATGGGGAGC ACACCGACTT GCATTGCTGC CCTCATATGC AAGTCATCAC |
| 1021 | CACTCTCTAG AAGCTTGGGC TGAAATTGTG CAGGCGTCTC CACACCCCCA TCTCATCCCG |
| 1081 | CATGATCTCC TCGCCGGCAG GGACCGTCTC GGGTTCCTAG CGAACCCCGA CTTGGTCCGC |
| 1141 | AGAAGCCGCG CGCCGCCCAC CCTCCGGCCT TCCCCCAGGC GAGGCCTCTC AGTACCCGAG |
| 1201 | GCTCCCTTTT CTCGAGCCCG CAGCGGCAGC GCTCCCAGCG GGTCCCCGGG AAGGAGACAG |
| 1261 | CTCGGGTACT GAGGGCGGGA AAGCAAGGAA GAGGCCAGAT CCCCATCCCT TGTCCCTGCG |
| 1321 | CCGCCGCCGC CGCCGCCGCC GCCGGGAAGC CGAATTCCGG GGCCCGGATG CAGGCAATTC |
| 1381 | CACCAGTCGC TAGAGGCGAA AGCCCGACAC CCAGCTTCGG TCAGAGAAAT GAGAGGGAAA |
| 1441 | GTAAAAATGC GTCGAGCTCT GAGGAGAGCC CCCGCTTCTA CCCGCGCCTC TTCCCGGCAG |
| 1501 | CCGAACCCCA AACAGCCACC CGCCAGGATG CCGCCTCCTC ACTCACCCAC TCGCCACCGC |
| 1561 | CTGCGCCTCC GCCGCCGCGG GCGCAGGCAC CGCAACCGCA GCCCCGCCCC GGGCCCGCCC |
| 1621 | CCGGGCCCGC CCCGACCACG AGCGGCCGCA GGCCCCGGCC CCGGCCCCGG CCCCGGCCCC |
| 1681 | GGCCCCGGCC CCGGCCCCGG CCCCGGCCCC GGCCCCGGCC CCGGCCCCGG CCCCGGCCCC |
| 1741 | GGCCCCGGCC CCGGCCCCGG CCCCGGCCCC GGCCCCGGCC CCGGCCCCGG CCCCGGCCCC |
| 1801 | GGCCCCGGCC CCGGCCCCGG CCCCGGCCCC GGCCCCGGCC CCGGCCCCGG CCCCGGCCCC |
| 1861 | GGCCCCGGCC CCGGCCCCGG CCCCGGCCCC GGCCCCGGCC CCGGCCCCGG CCCCGGCCCC |
| 1921 | GGCCCCGGCC CCGGCCCCGG CCCCGGCCCC GGCCCCGGCC CCGGCCCCGG CCCCGGCCCC |
| 1981 | GGCCCCGGCC CCGGCCCCGG CCCCGGCCCC GGCCCCGGCC CCGGCCCCGG CCCCGGCCCC |
| 2041 | GGCCCCGGCC CCGGCCCCGG CCCCGGCCCC GGCCCCGGCC CCGGCCCCGG CCCCGGCCCC |
| 2101 | GGCCCCGGCC CCGGCCCCGG CCCCGGCCCC GGCCCCGGCC CCGGCCCCGG CCCCGGCCCC |
| 2161 | GGCCCCGGCC CCGGCCCCGG CCCCGGCCCC GGCCCCGGCC CCGGCCACTA GTCAGCTGGA |
| 2221 | ATTGGCCGCT GGTCTTCACA CTCGAAGATT TCGTTGGGGA CTGGCGACAG ACAGCCGGCT |
| 2281 | ACAACCTGGA CCAAGTCCTT GAACAGGGAG GTGTGTCCAG TTTGTTTCAG AATCTCGGGG |
| 2341 | TGTCCGTAAC TCCGATCCAA AGGATTGTCC TGAGCGGTGA AAATGGGCTG AAGATCGACA |
| 2401 | TCCATGTCAT CATCCCGTAT GAAGGTCTGA GCGGCGACCA AATGGGCCAG ATCGAAAAAA |
| 2461 | TTTTTAAGGT GGTGTACCCT GTGGATGATC ATCACTTTAA GGTGATCCTG CACTATGGCA |
| 2521 | CACTGGTAAT CGACGGGGTT ACGCCGAACA TGATCGACTA TTTCGGACGG CCGTATGAAG |
| 2581 | GCATCGCCGT GTTCGACGGC AAAAAGATCA CTGTAACAGG GACCCTGTGG AACGGCAACA |
| 2641 | AAATTATCGA CGAGCGCCTG ATCAACCCCG ACGGCTCCCT GCTGTTCCGA GTAACCATCA |
| 2701 | ACGGAGTGAC CGGCTGGCGG CTGTGCGAAC GCATTCTGGC GTAATTCTAG AGTCGGGGCG |
| 2761 | GCCGGCCGCT TCGAGCAGAC ATGATAAGAT ACATTGATGA GTTTGGACAA ACCACAACTA |
| 2821 | GAATGCAGTG AAAAAAATGC TTTATTTGTG AAATTTGTGA TGCTATTGCT TTATTTGTAA |
| 2881 | CCATTATAAG CTGCAATAAA CAAGTTAACA ACAACAATTG CATTCATTTT ATGTTTCAGG |
| 2941 | TTCAGGGGGA GGTGTGGGAG GTTTTTTAAA GCAAGTAAAA CCTCTACAAA TGTGGTAAAA |
| 3001 | TCGATAAGGA TCTGAACGAT GGAGCGGAGA ATGGGCGGAA CTGGGCGGAG TTAGGGGCGG |
| 3061 | GATGGGCGGA GTTAGGGGCG GGACTATGGT TGCTGACTAA TTGAGATGCA TGCTTTGCAT |
| 3121 | ACTTCTGCCT GCTGGGGAGC CTGGGGACTT TCCACACCTG GTTGCTGACT AATTGAGATG |
| 3181 | CATGCTTTGC ATACTTCTGC CTGCTGGGGA GCCTGGGGAC TTTCCACACC CTAACTGACA |
| 3241 | CACATTCCAC AGCGGATCCG TCGACCGATG CCCTTGAGAG CCTTCAACCC AGTCAGCTCC |
| 3301 | TTCCGGTGGG CGCGGGGCAT GACTATCGTC GCCGCACTTA TGACTGTCTT CTTTATCATG |
| 3361 | CAACTCGTAG GACAGGTGCC GGCAGCGCTG TTCCGCTTCC TCGCTCACTG ACTCGCTGCG |
| 3421 | CTCGGTCGTT CGGCTGCGGC GAGCGGTATC AGCTCACTCA AAGGCGGTAA TACGGTTATC |
| 3481 | CACAGAATCA GGGGATAACG CAGGAAAGAA CATGTGAGCA AAAGGCCAGC AAAAGGCCAG |
| 3541 | GAACCGTAAA AAGGCCGCGT TGCTGGCGTT TTTCCATAGG CTCCGCCCCC CTGACGAGCA |
| 3601 | TCACAAAAAT CGACGCTCAA GTCAGAGGTG GCGAAACCCG ACAGGACTAT AAAGATACCA |
| 3661 | GGCGTTTCCC CCTGGAAGCT CCCTCGTGCG CTCTCCTGTT CCGACCCTGC CGCTTACCGG |
| 3721 | ATACCTGTCC GCCTTTCTCC CTTCGGGAAG CGTGGCGCTT TCTCATAGCT CACGCTGTAG |
| 3781 | GTATCTCAGT TCGGTGTAGG TCGTTCGCTC CAAGCTGGGC TGTGTGCACG AACCCCCCGT |
| 3841 | TCAGCCCGAC CGCTGCGCCT TATCCGGTAA CTATCGTCTT GAGTCCAACC CGGTAAGACA |
| 3901 | CGACTTATCG CCACTGGCAG CAGCCACTGG TAACAGGATT AGCAGAGCGA GGTATGTAGG |
| 3961 | CGGTGCTACA GAGTTCTTGA AGTGGTGGCC TAACTACGGC TACACTAGAA GAACAGTATT |
| 4021 | TGGTATCTGC GCTCTGCTGA AGCCAGTTAC CTTCGGAAAA AGAGTTGGTA GCTCTTGATC |
| 4081 | CGGCAAACAA ACCACCGCTG GTAGCGGTGG TTTTTTTGTT TGCAAGCAGC AGATTACGCG |
| 4141 | CAGAAAAAAA GGATCTCAAG AAGATCCTTT GATCTTTTCT ACGGGGTCTG ACGCTCAGTG |
| 4201 | GAACGAAAAC TCACGTTAAG GGATTTTGGT CATGAGATTA TCAAAAAGGA TCTTCACCTA |
| 4261 | GATCCTTTTA AATTAAAAAT GAAGTTTTAA ATCAATCTAA AGTATATATG AGTAAACTTG |
| 4321 | GTCTGACAGT TACCAATGCT TAATCAGTGA GGCACCTATC TCAGCGATCT GTCTATTTCG |
| 4381 | TTCATCCATA GTTGCCTGAC TCCCCGTCGT GTAGATAACT ACGATACGGG AGGGCTTACC |
| 4441 | ATCTGGCCCC AGTGCTGCAA TGATACCGCG AGACCCACGC TCACCGGCTC CAGATTTATC |
| 4501 | AGCAATAAAC CAGCCAGCCG GAAGGGCCGA GCGCAGAAGT GGTCCTGCAA CTTTATCCGC |
| 4561 | CTCCATCCAG TCTATTAATT GTTGCCGGGA AGCTAGAGTA AGTAGTTCGC CAGTTAATAG |
| 4621 | TTTGCGCAAC GTTGTTGCCA TTGCTACAGG CATCGTGGTG TCACGCTCGT CGTTTGGTAT |
| 4681 | GGCTTCATTC AGCTCCGGTT CCCAACGATC AAGGCGAGTT ACATGATCCC CCATGTTGTG |
| 4741 | CAAAAAAGCG GTTAGCTCCT TCGGTCCTCC GATCGTTGTC AGAAGTAAGT TGGCCGCAGT |
| 4801 | GTTATCACTC ATGGTTATGG CAGCACTGCA TAATTCTCTT ACTGTCATGC CATCCGTAAG |
| 4861 | ATGCTTTTCT GTGACTGGTG AGTACTCAAC CAAGTCATTC TGAGAATAGT GTATGCGGCG |
| 4921 | ACCGAGTTGC TCTTGCCCGG CGTCAATACG GGATAATACC GCGCCACATA GCAGAACTTT |
| 4981 | AAAAGTGCTC ATCATTGGAA AACGTTCTTC GGGGCGAAAA CTCTCAAGGA TCTTACCGCT |
| 5041 | GTTGAGATCC AGTTCGATGT AACCCACTCG TGCACCCAAC TGATCTTCAG CATCTTTTAC |
| 5101 | TTTCACCAGC GTTTCTGGGT GAGCAAAAAC AGGAAGGCAA AATGCCGCAA AAAAGGGAAT |
| 5161 | AAGGGCGACA CGGAAATGTT GAATACTCAT ACTCTTCCTT TTTCAATATT ATTGAAGCAT |
| 5221 | TTATCAGGGT TATTGTCTCA TGAGCGGATA CATATTTGAA TGTATTTAGA AAAATAAACA |
| 5281 | AATAGGGGTT CCGCGCACAT TTCCCCGAAA AGTGCCACCT GACGCGCCCT GTAGCGGCGC |
| 5341 | ATTAAGCGCG GCGGGTGTGG TGGTTACGCG CAGCGTGACC GCTACACTTG CCAGCGCCCT |
| 5401 | AGCGCCCGCT CCTTTCGCTT TCTTCCCTTC CTTTCTCGCC ACGTTCGCCG GCTTTCCCCG |
| 5461 | TCAAGCTCTA AATCGGGGGC TCCCTTTAGG GTTCCGATTT AGTGCTTTAC GGCACCTCGA |
| 5521 | CCCCAAAAAA CTTGATTAGG GTGATGGTTC ACGTAGTGGG CCATCGCCCT GATAGACGGT |
| 5581 | TTTTCGCCCT TTGACGTTGG AGTCCACGTT CTTTAATAGT GGACTCTTGT TCCAAACTGG |
| 5641 | AACAACACTC AACCCTATCT CGGTCTATTC TTTTGATTTA TAAGGGATTT TGCCGATTTC |
| 5701 | GGCCTATTGG TTAAAAAATG AGCTGATTTA ACAAAAATTT AACGCGAATT TTAACAAAAT |
| 5761 | ATTAACGCTT ACAATTTGCC ATTCGCCATT CAGGCTGCGC AACTGTTGGG AAGGGCGATC |
| 5821 | GGTGCGGGCC TCTTCGCTAT TACGCCAGCC CAAGCTACCA TGATAAGTAA GTAATATTAA |
| 5881 | GGTACGGGAG GTACTGGCCG CAATAAAATA TCTTTATTTT CATTACATCT GTGTGTTGGT |
| 5941 | TTTTTGTGTG AATCGATAGT ACTAACATAC GCTCTCCATC AAAACAAAAC GAAACAAAAC |
| 6001 | AAACTAGCAA AATAGGCTGT CCCCAGTGCA AGTGCAGGTG CCAGAACATT TCTCTATCGA |
| 6061 | TAGGTACCGA GCTCTTACGC GTGCTAGCCC GGGCTCGAG |
| S0RNL Nanoluciferase reporter plasmid | |
| SEQ ID NO: 59 | |
| 1 | TAGTAATCAA TTACGGGGTC ATTAGTTCAT AGCCCATATA TGGAGTTCCG CGTTACATAA |
| 61 | CTTACGGTAA ATGGCCCGCC TGGCTGACCG CCCAACGACC CCCGCCCATT GACGTCAATA |
| 121 | ATGACGTATG TTCCCATAGT AACGCCAATA GGGACTTTCC ATTGACGTCA ATGGGTGGAG |
| 181 | TATTTACGGT AAACTGCCCA CTTGGCAGTA CATCAAGTGT ATCATATGCC AAGTCCGCCC |
| 241 | CCTATTGACG TCAATGACGG TAAATGGCCC GCCTGGCATT ATGCCCAGTA CATGACCTTA |
| 301 | CGGGACTTTC CTACTTGGCA GTACATCTAC GTATTAGTCA TCGCTATTAC CATGGTGATG |
| 361 | CGGTTTTGGC AGTACACCAA TGGGCGTGGA TAGCGGTTTG ACTCACGGGG ATTTCCAAGT |
| 421 | CTCCACCCCA TTGACGTCAA TGGGAGTTTG TTTTGGCACC AAAATCAACG GGACTTTCCA |
| 481 | AAATGTCGTA ATAACCCCGC CCCGTTGACG CAAATGGGCG GTAGGCGTGT ACGGTGGGAG |
| 541 | GTCTATATAA GCAGAGCTCG TTTAGTGAAC CGTCAGATCA CTAGAAGCTT TATTGCGGTA |
| 601 | GTTTATCACA GTTAAATTGC TAACGCAGTC AGTGCTTCTG ACACAACAGT CTCGAACTTA |
| 661 | AGCTGCAGAA GTTGGTCGTG AGGCACTGGG CAGGTAAGTA TCAAGGTTAC AAGACAGGTT |
| 721 | TAAGGAGACC AATAGAAACT GGGCTTGTCG AGACAGAGAA GACTCTTGCG TTTCTGATAG |
| 781 | GCACCTATTG GTCTTACTGA CATCCACTTT GCCTTTCTCT CCACAGGTGT CCACTCCCAG |
| 841 | TTCAATTACA GCTCTTAAGG CTAGAGTACT TAATACGACT CACTATAGGG ATATCTGCTT |
| 901 | ATCGATACCG TCGACCTCGA ATCACTAGTC AGCTGGAATT CCTCACAGTA CTCGCTGAGG |
| 961 | GTGAACAAGA AAAGACCTGA TAAAGATTAA CCAGAAGAAA ACAAGGAGGG AAACAACCGC |
| 1021 | AGCCTGTAGC AAGCTCTGGA ACTCAGGAGT CGCGCGCTAG GGGGCTCTGG CCGCTGGTCT |
| 1081 | TCACACTCGA AGATTTCGTT GGGGACTGGC GACAGACAGC CGGCTACAAC CTGGACCAAG |
| 1141 | TCCTTGAACA GGGAGGTGTG TCCAGTTTGT TTCAGAATCT CGGGGTGTCC GTAACTCCGA |
| 1201 | TCCAAAGGAT TGTCCTGAGC GGTGAAAATG GGCTGAAGAT CGACATCCAT GTCATCATCC |
| 1261 | CGTATGAAGG TCTGAGCGGC GACCAAATGG GCCAGATCGA AAAAATTTTT AAGGTGGTGT |
| 1321 | ACCCTGTGGA TGATCATCAC TTTAAGGTGA TCCTGCACTA TGGCACACTG GTAATCGACG |
| 1381 | GGGTTACGCC GAACATGATC GACTATTTCG GACGGCCGTA TGAAGGCATC GCCGTGTTCG |
| 1441 | ACGGCAAAAA GATCACTGTA ACAGGGACCC TGTGGAACGG CAACAAAATT ATCGACGAGC |
| 1501 | GCCTGATCAA CCCCGACGGC TCCCTGCTGT TCCGAGTAAC CATCAACGGA GTGACCGGCT |
| 1561 | GGCGGCTGTG CGAACGCATT CTGGCGTAAT TCTAGAGTCG GGGCGGCCGG CCGCTTCGAG |
| 1621 | CAGACATGAT AAGATACATT GATGAGTTTG GACAAACCAC AACTAGAATG CAGTGAAAAA |
| 1681 | AATGCTTTAT TTGTGAAATT TGTGATGCTA TTGCTTTATT TGTAACCATT ATAAGCTGCA |
| 1741 | ATAAACAAGT TAACAACAAC AATTGCATTC ATTTTATGTT TCAGGTTCAG GGGGAGGTGT |
| 1801 | GGGAGGTTTT TTAAAGCAAG TAAAACCTCT ACAAATGTGG TAAAATCGAT AAGGATCTGA |
| 1861 | ACGATGGAGC GGAGAATGGG CGGAACTGGG CGGAGTTAGG GGCGGGATGG GCGGAGTTAG |
| 1921 | GGGCGGGACT ATGGTTGCTG ACTAATTGAG ATGCATGCTT TGCATACTTC TGCCTGCTGG |
| 1981 | GGAGCCTGGG GACTTTCCAC ACCTGGTTGC TGACTAATTG AGATGCATGC TTTGCATACT |
| 2041 | TCTGCCTGCT GGGGAGCCTG GGGACTTTCC ACACCCTAAC TGACACACAT TCCACAGCGG |
| 2101 | ATCCGTCGAC CGATGCCCTT GAGAGCCTTC AACCCAGTCA GCTCCTTCCG GTGGGCGCGG |
| 2161 | GGCATGACTA TCGTCGCCGC ACTTATGACT GTCTTCTTTA TCATGCAACT CGTAGGACAG |
| 2221 | GTGCCGGCAG CGCTGTTCCG CTTCCTCGCT CACTGACTCG CTGCGCTCGG TCGTTCGGCT |
| 2281 | GCGGCGAGCG GTATCAGCTC ACTCAAAGGC GGTAATACGG TTATCCACAG AATCAGGGGA |
| 2341 | TAACGCAGGA AAGAACATGT GAGCAAAAGG CCAGCAAAAG GCCAGGAACC GTAAAAAGGC |
| 2401 | CGCGTTGCTG GCGTTTTTCC ATAGGCTCCG CCCCCCTGAC GAGCATCACA AAAATCGACG |
| 2461 | CTCAAGTCAG AGGTGGCGAA ACCCGACAGG ACTATAAAGA TACCAGGCGT TTCCCCCTGG |
| 2521 | AAGCTCCCTC GTGCGCTCTC CTGTTCCGAC CCTGCCGCTT ACCGGATACC TGTCCGCCTT |
| 2581 | TCTCCCTTCG GGAAGCGTGG CGCTTTCTCA TAGCTCACGC TGTAGGTATC TCAGTTCGGT |
| 2641 | GTAGGTCGTT CGCTCCAAGC TGGGCTGTGT GCACGAACCC CCCGTTCAGC CCGACCGCTG |
| 2701 | CGCCTTATCC GGTAACTATC GTCTTGAGTC CAACCCGGTA AGACACGACT TATCGCCACT |
| 2761 | GGCAGCAGCC ACTGGTAACA GGATTAGCAG AGCGAGGTAT GTAGGCGGTG CTACAGAGTT |
| 2821 | CTTGAAGTGG TGGCCTAACT ACGGCTACAC TAGAAGAACA GTATTTGGTA TCTGCGCTCT |
| 2881 | GCTGAAGCCA GTTACCTTCG GAAAAAGAGT TGGTAGCTCT TGATCCGGCA AACAAACCAC |
| 2941 | CGCTGGTAGC GGTGGTTTTT TTGTTTGCAA GCAGCAGATT ACGCGCAGAA AAAAAGGATC |
| 3001 | TCAAGAAGAT CCTTTGATCT TTTCTACGGG GTCTGACGCT CAGTGGAACG AAAACTCACG |
| 3061 | TTAAGGGATT TTGGTCATGA GATTATCAAA AAGGATCTTC ACCTAGATCC TTTTAAATTA |
| 3121 | AAAATGAAGT TTTAAATCAA TCTAAAGTAT ATATGAGTAA ACTTGGTCTG ACAGTTACCA |
| 3181 | ATGCTTAATC AGTGAGGCAC CTATCTCAGC GATCTGTCTA TTTCGTTCAT CCATAGTTGC |
| 3241 | CTGACTCCCC GTCGTGTAGA TAACTACGAT ACGGGAGGGC TTACCATCTG GCCCCAGTGC |
| 3301 | TGCAATGATA CCGCGAGACC CACGCTCACC GGCTCCAGAT TTATCAGCAA TAAACCAGCC |
| 3361 | AGCCGGAAGG GCCGAGCGCA GAAGTGGTCC TGCAACTTTA TCCGCCTCCA TCCAGTCTAT |
| 3421 | TAATTGTTGC CGGGAAGCTA GAGTAAGTAG TTCGCCAGTT AATAGTTTGC GCAACGTTGT |
| 3481 | TGCCATTGCT ACAGGCATCG TGGTGTCACG CTCGTCGTTT GGTATGGCTT CATTCAGCTC |
| 3541 | CGGTTCCCAA CGATCAAGGC GAGTTACATG ATCCCCCATG TTGTGCAAAA AAGCGGTTAG |
| 3601 | CTCCTTCGGT CCTCCGATCG TTGTCAGAAG TAAGTTGGCC GCAGTGTTAT CACTCATGGT |
| 3661 | TATGGCAGCA CTGCATAATT CTCTTACTGT CATGCCATCC GTAAGATGCT TTTCTGTGAC |
| 3721 | TGGTGAGTAC TCAACCAAGT CATTCTGAGA ATAGTGTATG CGGCGACCGA GTTGCTCTTG |
| 3781 | CCCGGCGTCA ATACGGGATA ATACCGCGCC ACATAGCAGA ACTTTAAAAG TGCTCATCAT |
| 3841 | TGGAAAACGT TCTTCGGGGC GAAAACTCTC AAGGATCTTA CCGCTGTTGA GATCCAGTTC |
| 3901 | GATGTAACCC ACTCGTGCAC CCAACTGATC TTCAGCATCT TTTACTTTCA CCAGCGTTTC |
| 3961 | TGGGTGAGCA AAAACAGGAA GGCAAAATGC CGCAAAAAAG GGAATAAGGG CGACACGGAA |
| 4021 | ATGTTGAATA CTCATACTCT TCCTTTTTCA ATATTATTGA AGCATTTATC AGGGTTATTG |
| 4081 | TCTCATGAGC GGATACATAT TTGAATGTAT TTAGAAAAAT AAACAAATAG GGGTTCCGCG |
| 4141 | CACATTTCCC CGAAAAGTGC CACCTGACGC GCCCTGTAGC GGCGCATTAA GCGCGGCGGG |
| 4201 | TGTGGTGGTT ACGCGCAGCG TGACCGCTAC ACTTGCCAGC GCCCTAGCGC CCGCTCCTTT |
| 4261 | CGCTTTCTTC CCTTCCTTTC TCGCCACGTT CGCCGGCTTT CCCCGTCAAG CTCTAAATCG |
| 4321 | GGGGCTCCCT TTAGGGTTCC GATTTAGTGC TTTACGGCAC CTCGACCCCA AAAAACTTGA |
| 4381 | TTAGGGTGAT GGTTCACGTA GTGGGCCATC GCCCTGATAG ACGGTTTTTC GCCCTTTGAC |
| 4441 | GTTGGAGTCC ACGTTCTTTA ATAGTGGACT CTTGTTCCAA ACTGGAACAA CACTCAACCC |
| 4501 | TATCTCGGTC TATTCTTTTG ATTTATAAGG GATTTTGCCG ATTTCGGCCT ATTGGTTAAA |
| 4561 | AAATGAGCTG ATTTAACAAA AATTTAACGC GAATTTTAAC AAAATATTAA CGCTTACAAT |
| 4621 | TTGCCATTCG CCATTCAGGC TGCGCAACTG TTGGGAAGGG CGATCGGTGC GGGCCTCTTC |
| 4681 | GCTATTACGC CAGCCCAAGC TACCATGATA AGTAAGTAAT ATTAAGGTAC GGGAGGTACT |
| 4741 | GGCCGCAATA AAATATCTTT ATTTTCATTA CATCTGTGTG TTGGTTTTTT GTGTGAATCG |
| 4801 | ATAGTACTAA CATACGCTCT CCATCAAAAC AAAACGAAAC AAAACAAACT AGCAAAATAG |
| 4861 | GCTGTCCCCA GTGCAAGTGC AGGTGCCAGA ACATTTCTCT ATCGATAGGT ACCGAGCTCT |
| 4921 | TACGCGTGCT AGCCCGGGCT CGAG |
| Example wild type Cas13d polypeptide sequence (from Ruminococcus flavefaciens) | |
| SEQ ID NO: 64 | |
| 1 | IEKKKSFAKG MGVKSTLVSG SKVYMTTFAE GSDARLEKIV EGDSIRSVNE GEAFSAEMAD |
| 61 | KNAGYKIGNA KFSHPKGYAW ANNPLYTGPV QQDMLGLKET LEKRYFGESA DGNDNICIQV |
| 121 | IHNILDIEKI LAEYITNMYA VNNISGLDKD IIGFGKFSTV YTYDEFKDPE HHRAAFNNND |
| 181 | KLINAIKAQY DEFDNFLDNP RLGYFGQAFF SKEGRNYIIN YGNECYDILA LLSGLAHWVV |
| 241 | ANNEEESRIS RTWLYNLDKN LDNEYISTLN YLYDRITNEL TNSFSKNSMN VNYIAETLGI |
| 301 | NPAEFAEQYF RFSIMKEQKN LGFNITKLRE VMLDRKDMSE IRKNHKVFDS IRTKVYTMMD |
| 361 | FVIYRYYIEE DAKVAMNKSL PDNEKSLSEK DIFVINLRGS FNDDQKDALY YDEANRIWRK |
| 421 | LENIMHNIKE FRGNKTREYK KKDAPRLPRI LPAGRDVSAF SKLMYALTMF LDGKEINDLL |
| 481 | TTLINKFDNI QSFLKVMPLI GVNAKFVEEY AFFKDSAKIA DELRLIKSFA RMGEPIADAR |
| 541 | RAMYIDAIRI LGTNLSYDEL KALADTFSLD ENGNKLKKGK HGMRNFIINN VISNKRFHYL |
| 601 | IRYGDPAHLH EIAKNEAVVK FVLGRIADIQ KKQGQNGKNQ IDRYYETCIG KDKGKSVSEK |
| 661 | VDALTKIITG MNYDQFDKKR SVIEDTGREN AEREKFKKII SLYLTVIYHI LKNIVNINAR |
| 721 | YVIGFHCVER DAQLYKEKGY DINLKKLEEK GFSSVTKLCA GIDETAPDKR KDVEKEMAER |
| 781 | AKESIDSLES ANPKLYANYI KYSDEKKAEE FTRQINREKA KTALNAYLRN TKWNVIIRED |
| 841 | LLRIDNKTCT LFANKAVALE VARYVHAYIN DIAEVNSYFQ LYHYIMQRII MNERYEKSSG |
| 901 | KVSEYFDAVN DEKKYNDRLL KLLCVPFGYC IPRFKNLSIE ALFDRNEMKF DKEKKSGNS |
| Example Cas13Rx polypeptide sequence | |
| SEQ ID NO: 65 | |
| 1 | MSPKKKRKVE ASIEKKKSFA KGMGVKSTLV SGSKVYMTTF AEGSDARLEK IVEGDSIRSV |
| 61 | NEGEAFSAEM ADKNAGYKIG NAKFSHPKGY AVVANNPLYT GPVQQDMLGL KETLEKRYFG |
| 121 | ESADGNDNIC IQVIHNILDI EKILAEYITN AAYAVNNISG LDKDIIGFGK FSTVYTYDEF |
| 181 | KDPEHHRAAF NNNDKLINAI KAQYDEFDNF LDNPRLGYFG QAFFSKEGRN YIINYGNECY |
| 241 | DILALLSGLR HWVVHNNEEE SRISRTWLYN LDKNLDNEYI STLNYLYDRI TNELTNSFSK |
| 301 | NSAANVNYIA ETLGINPAEF AEQYFRFSIM KEQKNLGFNI TKLREVMLDR KDMSEIRKNH |
| 361 | KVFDSIRTKV YTMMDFVIYR YYIEEDAKVA AANKSLPDNE KSLSEKDIFV INLRGSFNDD |
| 421 | QKDALYYDEA NRIWRKLENI MHNIKEFRGN KTREYKKKDA PRLPRILPAG RDVSAFSKLM |
| 481 | YALTMFLDGK EINDLLTTLI NKFDNIQSFL KVMPLIGVNA KFVEEYAFFK DSAKIADELR |
| 541 | LIKSFARMGE PIADARRAMY IDAIRILGTN LSYDELKALA DTFSLDENGN KLKKGKHGMR |
| 601 | NFIINNVISN KRFHYLIRYG DPAHLHEIAK NEAVVKFVLG RIADIQKKQG QNGKNQIDRY |
| 661 | YETCIGKDKG KSVSEKVDAL TKIITGMNYD QFDKKRSVIE DIGRENAERE KFKKIISLYL |
| 721 | TVIYHILKNI VNINARYVIG FHCVERDAQL YKEKGYDINL KKLEEKGFSS VTKLCAGIDE |
| 781 | TAPDKRKDVE KEMAERAKES IDSLESANPK LYANYIKYSD EKKAEEFTRQ INREKAKTAL |
| 841 | NAYLRNTKWN VIIREDLLRI DNKTCTLFRN KAVHLEVARY VHAYINDIAE VNSYFQLYHY |
| 901 | IMQRIIMNER YEKSSGKVSE YFDAVNDEKK YNDRLLKLLC VPFGYCIPRF KNLSIEALFD |
| 961 | RNEAAKFDKE KKKVSGNSGS GPKKKRKVAA AYPYDVPDYA |

1. A composition comprising:
(i) a nucleic acid sequence encoding a CasRx/Cas13d polypeptide; and
(ii) one or more guide RNAs that binds specifically to a target sequence in C9orf72 RNA.

2. A composition according to claim 1, wherein the one or more guide RNAs bind to the CasRx/Cas13d polypeptide and directs specific cleavage and/or degradation of C9orf72 RNA.

3. A composition according to claim 1, wherein the target sequence is present in a sense C9orf72 transcript, and/or the one more guide RNAs direct CasRx/Cas13d-mediated cleavage and/or degradation of a sense C9orf72 transcript; preferably wherein the target sequence corresponds to or is within base pairs 150-400, 150-350, 200-350, or 200-320 of SEQ ID NO: 56.

4. A composition according to claim 1, wherein the target sequence is present in an antisense C9orf72 transcript, and/or the one or more guide RNAs direct CasRx/Cas13d-mediated cleavage and/or degradation of an antisense C9orf72 transcript; preferably wherein the target sequence is complementary to a sequence within base pairs 350-700, 350-650, 400-700, 350-600, 400-650, 400-600, or 410-575 of SEQ ID NO: 56.

5. A composition according to claim 1, wherein the composition comprises a first guide RNA that binds specifically to a target sequence in a sense C9orf72 transcript, and a second guide RNA that binds specifically to a target sequence in an antisense C9orf72 transcript; and/or wherein the guide RNAs direct specific cleavage and/or degradation of sense and antisense C9orf72 transcripts.

6. A composition according to claim 1, wherein the target sequence is 5′ to a hexanucleotide repeat sequence in a sense C9orf72 transcript.

7. A composition according to claim 1, wherein the target sequence corresponds to a sequence 5′ of a hexanucleotide repeat sequence in intron 1 of C9orf72.

8. A composition according to claim 6, wherein the hexanucleotide repeat comprises the sequence (G₄C₂)ₙ.

9. A composition according to claim 1, wherein the one or more guide RNAs preferentially bind to and/or directs specific cleavage and/or degradation of C9orf72 RNA variants 1 and/or 3.

10. A composition according to claim 1, wherein the one or more guide RNAs do not bind to and/or do not cleave and/or do not degrade C9orf72 transcript variant 2.

11. A composition according to claim 1, wherein the one or more guide RNAs comprise any one of SEQ ID NO:s 1-30, preferably any one of SEQ ID NO:s 1-3, or 22-30.

12. A composition according to claim 1, wherein the target sequence is 5′ to a hexanucleotide repeat sequence in an antisense C9orf72 transcript.

13. A composition according to claim 12, wherein the hexanucleotide repeat comprises the sequence (C₄G₂)ₙ.

14. A composition according to claim 1, wherein the one or more guide RNAs comprise any one of SEQ ID NO:s 31-45, preferably any one of SEQ ID NO: s 31-33, or 37-45.

15. A guide RNA that binds specifically to a target sequence in C9orf72 RNA, wherein the guide RNA is capable of binding to a CasRx/Cas13d polypeptide and directing specific cleavage and/or degradation of C9orf72 RNA.

16. A guide RNA according to claim 15, wherein the guide RNA comprises a spacer sequence complementary to, or capable of specifically hybridizing to, the target sequence.

17. A guide RNA according to claim 16, wherein the spacer sequence is selected from any one of SEQ ID NO:s 1, 22, 25, 28, 31, 37, 40 or 43.

18. A guide RNA according to claim 15, wherein the guide RNA comprises a direct repeat sequence capable of binding to the CasRx/Cas13d polypeptide, preferably wherein the direct repeat sequence comprises SEQ ID NO:46 or SEQ ID NO: 47.

19. A complex comprising:
(i) a CasRx/Cas13d polypeptide; and
(ii) one or more guide RNAs as defined in claim 15 bound to the CasRx/Cas13d polypeptide.

20. A vector comprising the composition of claim 1.

21. A vector according to claim 20, wherein the vector is an adeno-associated virus (AAV) or a lentivirus.

22. A cell comprising the composition of claim 1.

23. A pharmaceutical composition comprising the composition of claim 1, and one or more pharmaceutically acceptable excipients, carriers or diluents.

24-26. (canceled)

27. A method of cleaving and/or degrading C9orf72 RNA in a preparation or cell, comprising contacting the preparation or cell with a composition according to claim 1.

28. A method according to claim 27, wherein the method selectively degrades C9orf72 pre-RNA that comprises a hexanucleotide repeat expansion.

29. A method of preventing or treating a C9orf72-mediated disease, disorder or condition in a subject in need thereof, wherein the method comprises administering to the subject a therapeutically effective amount of a composition according to claim 1.

30. A method according to claim 29, wherein the C9orf72-mediated disease, disorder or condition is a neurodegenerative disorder, preferably wherein the neurodegenerative disorder is frontotemporal dementia (FTD) or amyotrophic lateral sclerosis (ALS).


SEQUENCE LISTING (77 sequences)

SEQ ID NO: 1 - DNA, 30 bp, Artificial Sequence
  Guide 1 (sense) custom spacer
cttgttcaccctcagcgagtactgtgagag

SEQ ID NO: 2 - DNA, 66 bp, Artificial Sequence
  Guide 1 (sense) Pre-gRNA + spacer
caagtaaacccctaccaactggtcggggtttgaaaccttgttcaccctcagcgagtactgtgagag

SEQ ID NO: 3 - DNA, 52 bp, Artificial Sequence
  Guide 1 (sense) mature gRNA + spacer
aacccctaccaactggtcggggtttgaaaccttgttcaccctcagcgagtac

SEQ ID NO: 4 - DNA, 30 bp, Artificial Sequence
  Guide 2 (sense) custom spacer
caggtcttttcttgttcaccctcagcgagt

SEQ ID NO: 5 - DNA, 66 bp, Artificial Sequence
  Guide 2 (sense) pre-gRNA + spacer
caagtaaacccctaccaactggtcggggtttgaaaccaggtcttttcttgttcaccctcagcgagt

SEQ ID NO: 6 - DNA, 52 bp, Artificial Sequence
  Guide 2 (sense) mature gRNA + spacer
aacccctaccaactggtcggggtttgaaaccaggtcttttcttgttcaccct

SEQ ID NO: 7 - DNA, 30 bp, Artificial Sequence
  Guide 3 (sense) custom spacer
taatctttatcaggtcttttcttgttcacc

SEQ ID NO: 8 - DNA, 66 bp, Artificial Sequence
  Guide 3 (sense) pre-gRNA + spacer
caagtaaacccctaccaactggtcggggtttgaaactaatctttatcaggtcttttcttgttcacc

SEQ ID NO: 9 - DNA, 52 bp, Artificial Sequence
  Guide 3 (sense) mature gRNA + spacer
aacccctaccaactggtcggggtttgaaactaatctttatcaggtcttttct

SEQ ID NO: 10 - DNA, 30 bp, Artificial Sequence
  Guide 4 (sense) custom spacer
ttcttctggttaatctttatcaggtctttt

SEQ ID NO: 11 - DNA, 66 bp, Artificial Sequence
  Guide 4 (sense) pre-gRNA + spacer
caagtaaacccctaccaactggtcggggtttgaaacttcttctggttaatctttatcaggtctttt

SEQ ID NO: 12 - DNA, 52 bp, Artificial Sequence
  Guide 4 (sense) mature gRNA + spacer
aacccctaccaactggtcggggtttgaaacttcttctggttaatctttatca

SEQ ID NO: 13 - DNA, 30 bp, Artificial Sequence
  Guide 5 (sense) custom spacer
cctccttgttttcttctggttaatctttat

SEQ ID NO: 14 - DNA, 66 bp, Artificial Sequence
  Guide 5 (sense) pre-gRNA + spacer
caagtaaacccctaccaactggtcggggtttgaaaccctccttgttttcttctggttaatctttat

SEQ ID NO: 15 - DNA, 52 bp, Artificial Sequence
  Guide 5 (sense) mature gRNA + spacer
aacccctaccaactggtcggggtttgaaaccctccttgttttcttctggtta

SEQ ID NO: 16 - DNA, 30 bp, Artificial Sequence
  Guide 6 (sense) custom spacer
cggttgtttccctccttgttttcttctggt

SEQ ID NO: 17 - DNA, 66 bp, Artificial Sequence
  Guide 6 (sense) pre-gRNA + spacer
caagtaaacccctaccaactggtcggggtttgaaaccggttgtttccctccttgttttcttctggt

SEQ ID NO: 18 - DNA, 52 bp, Artificial Sequence
  Guide 6 (sense) mature gRNA + spacer
aacccctaccaactggtcggggtttgaaaccggttgtttccctccttgtttt

SEQ ID NO: 19 - DNA, 30 bp, Artificial Sequence
  Guide 7 (sense) custom spacer
ctacaggctgcggttgtttccctccttgtt

SEQ ID NO: 20 - DNA, 66 bp, Artificial Sequence
  Guide 7 (sense) pre-gRNA + spacer
caagtaaacccctaccaactggtcggggtttgaaacctacaggctgcggttgtttccctccttgtt

SEQ ID NO: 21 - DNA, 52 bp, Artificial Sequence
  Guide 7 (sense) mature gRNA + spacer
aacccctaccaactggtcggggtttgaaacctacaggctgcggttgtttccc

SEQ ID NO: 22 - DNA, 30 bp, Artificial Sequence
  Guide 8 (sense) custom spacer
ccagagcttgctacaggctgcggttgtttc

SEQ ID NO: 23 - DNA, 66 bp, Artificial Sequence
  Guide 8 (sense) pre-gRNA + spacer
caagtaaacccctaccaactggtcggggtttgaaacccagagcttgctacaggctgcggttgtttc

SEQ ID NO: 24 - DNA, 52 bp, Artificial Sequence
  Guide 8 (sense) mature gRNA + spacer
aacccctaccaactggtcggggtttgaaacccagagcttgctacaggctgcg

SEQ ID NO: 25 - DNA, 30 bp, Artificial Sequence
  Guide 9 (sense) custom spacer
ctcctgagttccagagcttgctacaggctg

SEQ ID NO: 26 - DNA, 66 bp, Artificial Sequence
  Guide 9 (sense) pre-gRNA + spacer
caagtaaacccctaccaactggtcggggtttgaaacctcctgagttccagagcttgctacaggctg

SEQ ID NO: 27 - DNA, 52 bp, Artificial Sequence
  Guide 9 (sense) mature gRNA + spacer
aacccctaccaactggtcggggtttgaaacctcctgagttccagagcttgct

SEQ ID NO: 28 - DNA, 30 bp, Artificial Sequence
  Guide 10 (sense) custom spacer
tagcgcgcgactcctgagttccagagcttg

SEQ ID NO: 29 - DNA, 66 bp, Artificial Sequence
  Guide 10 (sense) pre-gRNA + spacer
caagtaaacccctaccaactggtcggggtttgaaactagcgcgcgactcctgagttccagagcttg

SEQ ID NO: 30 - DNA, 52 bp, Artificial Sequence
  Guide 10 (sense) mature gRNA + spacer
aacccctaccaactggtcggggtttgaaactagcgcgcgactcctgagttcc

SEQ ID NO: 31 - DNA, 30 bp, Artificial Sequence
  Guide 11 (antisense) custom spacer
cgcaggcggtggcgagtgggtgagtgagga

SEQ ID NO: 32 - DNA, 66 bp, Artificial Sequence
  Guide 11 (antisense) pre-gRNA + spacer
caagtaaacccctaccaactggtcggggtttgaaaccgcaggcggtggcgagtgggtgagtgagga

SEQ ID NO: 33 - DNA, 52 bp, Artificial Sequence
  Guide 11 (antisense) mature gRNA + spacer
aacccctaccaactggtcggggtttgaaaccgcaggcggtggcgagtgggtg

SEQ ID NO: 34 - DNA, 30 bp, Artificial Sequence
  Guide 12 (antisense) custom spacer
tgcgcccgcggcggcggaggcgcaggcggt

SEQ ID NO: 35 - DNA, 66 bp, Artificial Sequence
  Guide 12 (antisense) pre-gRNA + spacer
caagtaaacccctaccaactggtcggggtttgaaactgcgcccgcggcggcggaggcgcaggcggt

SEQ ID NO: 36 - DNA, 52 bp, Artificial Sequence
  Guide 12 (antisense) mature gRNA + spacer
aacccctaccaactggtcggggtttgaaactgcgcccgcggcggcggaggcg

SEQ ID NO: 37 - DNA, 30 bp, Artificial Sequence
  Guide 13 (antisense) custom spacer
ttaactttccctctcatttctctgaccgaa

SEQ ID NO: 38 - DNA, 66 bp, Artificial Sequence
  Guide 13 (antisense) pre-gRNA + spacer
caagtaaacccctaccaactggtcggggtttgaaacttaactttccctctcatttctctgaccgaa

SEQ ID NO: 39 - DNA, 52 bp, Artificial Sequence
  Guide 13 (antisense) mature gRNA + spacer
aacccctaccaactggtcggggtttgaaacttaactttccctctcatttctc

SEQ ID NO: 40 - DNA, 30 bp, Artificial Sequence
  Guide 14 (antisense) custom spacer
ttcggctgccgggaagaggcgcgggtagaa

SEQ ID NO: 41 - DNA, 66 bp, Artificial Sequence
  Guide 14 (antisense) pre-gRNA + spacer
caagtaaacccctaccaactggtcggggtttgaaacttcggctgccgggaagaggcgcgggtagaa

SEQ ID NO: 42 - DNA, 52 bp, Artificial Sequence
  Guide 14 (antisense) mature gRNA + spacer
aacccctaccaactggtcggggtttgaaacttcggctgccgggaagaggcgc

SEQ ID NO: 43 - DNA, 30 bp, Artificial Sequence
  Guide 17 (antisense) custom spacer
tccctctcatttctctgaccgaagctgggt

SEQ ID NO: 44 - DNA, 66 bp, Artificial Sequence
  Guide 17 (antisense) pre-gRNA + spacer
caagtaaacccctaccaactggtcggggtttgaaactccctctcatttctctgaccgaagctgggt

SEQ ID NO: 45 - DNA, 53 bp, Artificial Sequence
  Guide 17 (antisense) mature gRNA + spacer
aacccctaccaactggtcggggtttgaaactccctctcatttctctgaccgaa

SEQ ID NO: 46 - DNA, 36 bp, Artificial Sequence
  Pre-gRNA direct repeat sequence
caagtaaacccctaccaactggtcggggtttgaaac

SEQ ID NO: 47 - DNA, 30 bp, Artificial Sequence
  Mature gRNA direct repeat sequence
aacccctaccaactggtcggggtttgaaac

SEQ ID NO: 48 - DNA, 31 bp, Artificial Sequence
  U6 gRNA PacI PCR Cloning Forward Primer
gcattaattaattacggttcctggccttttg

SEQ ID NO: 49 - DNA, 34 bp, Artificial Sequence
  U6 gRNA PacI PCR Cloning Reverse Primer
gcattaattaacgtaaggagaaaataccgcatca

SEQ ID NO: 50 - DNA, 20 bp, Artificial Sequence
  AS55RNL Seq Forward Primer
ttcctagcaaccccgacttg

SEQ ID NO: 51 - DNA, 22 bp, Artificial Sequence
  AS55RNL Seq Reverse Primer
ttggatcggagttacggacacc

SEQ ID NO: 52 - DNA, 21 bp, Artificial Sequence
  Ef1a Seq Forward Primer
tcaagcctcagacagtggttc

SEQ ID NO: 53 - DNA, 20 bp, Artificial Sequence
  U6 Seq Forward Primer
gactatcatatgcttaccgt

SEQ ID NO: 54 - DNA, 16 bp, Artificial Sequence
  RNA-FISH Locked Nucleic Acid (LNA) Probe Sense
ccccggccccggcccc

SEQ ID NO: 55 - DNA, 16 bp, Artificial Sequence
  RNA-FISH Locked Nucleic Acid (LNA) Probe Antisense
ggggccggggccgggg

SEQ ID NO: 56 - DNA, 27322 bp, Artificial Sequence
  Nucleotide sequence of C9orf72 gene
acgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctaggggccggggccggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccgggaagaggcgcgggtagaagcgggggctctcctcagagctcgacgcatttttactttccctctcatttctctgaccgaagctgggtgtcgggctttcgcctctagcgactggtggaattgcctgcatccgggccccgggcttcccggcggcggcggcggcggcggcggcgcagggacaagggatggggatctggcctcttccttgctttcccgccctcagtacccgagctgtctccttcccggggacccgctgggagcgctgccgctgcgggctcgagaaaagggagcctcgggtactgagaggcctcgcctgggggaaggccggagggtgggcggcgcgcggcttctgcggaccaagtcggggttcgctaggaacccgagacggtccctgccggcgaggagatcatgcgggatgagatgggggtgtggagacgcctgcacaatttcagcccaagcttctagagagtggtgatgacttgcatatgagggcagcaatgcaagtcggtgtgctccccattctgtgggacatgacctggttgcttcacagctccgagatgacacagacttgcttaaaggaagtgactattgtgacttgggcatcacttgactgatggtaatcagttgtctaaagaagtgcacagattacatgtccgtgtgctcattgggtctatctggccgcgttgaacaccaccaggctttgtattcagaaacaggagggaggtcctgcactttcccaggaggggtggccctttcagatgcaatcgagattgttaggctctgggagagtagttgcctggttgtggcagttggtaaatttctattcaaacagttgccatgcaccagttgttcacaacaagggtacgtaatctgtctggcattacttctacttttgtacaaaggatcaaaaaaaaaaaagatactgttaagatatgatttttctcagactttgggaaacttttaacataatctgtgaatatcacagaaacaagactatcatataggggatattaataacctggagtcagaatacttgaaatacggtgtcatttgacacgggcattgttgtcaccacctctgccaaggcctgccactttaggaaaaccctgaatcagttggaaactgctacatgctgatagtacatctgaaacaagaacgagagtaattaccacattccagattgttcactaagccagcatttacctgctccaggaaaaaattacaagcaccttatgaagttgataaaatattttgtttggctatgttggcactccacaatttgctttcagagaaacaaagtaaaccaaggaggacttctgtttttcaagtctgccctcgggttctattctacgttaattagatagttcccaggaggactaggttagcctacctattgtctgagaaacttggaactgtgagaaatggccagatagtgatatgaacttcaccttccagtcttccctgatgttgaagattgagaaagtgttgtgaactttctggtactgtaaacagttcactgtccttgaagtggtcctgggcagctcctgttgtggaaagtggacggtttaggatcctgcttctctttgggctgggagaaaataaacagcatggttacaagtattgagagccaggttggagaaggtggcttacacctgtaatgccagagctttgggaggcggaggcaagaggatcacttgaagccaggagttcaagctcaacctgggcaacgtagaccctgtctctacaaaaaattaaaaacttagccgggcgtggtgatgtgcacctgtagtcctagctacttgggaggctgaggcaggagggtcatttgagcccaagagtttgaagttaccgagagctatgatcctgccagtgcattccagcctggatgacaaaacgagaccctgtctctaaaaaacaagaagtgagggctttatgattgtagaattttcactacaatagcagtggaccaaccacctttctaaataccaatcagggaagagatggttgattttttaacagacgtttaaagaaaaagcaaaacctcaaacttagcactctactaacagttttagcagatgttaattaatgtaatcatgtctgcatgtatgggattatttccagaaagtgtattgggaaacctctcatgaaccctgtgagcaagccaccgtctcactcaatttgaatcttggcttccctcaaaagactggctaatgtttggtaactctctggagtagacagcactacatgtacgtaagataggtacataaacaactattggttttgagctgatttttttcagctgcatttgcatgtatggatttttctcaccaaagacgatgacttcaagtattagtaaaataattgtacagctctcctgattatacttctctgtgacatttcatttcccaggctatttcttttggtaggatttaaaactaagcaattcagtatgatctttgtccttcattttctttcttattctttttgtttgtttgtttgtttgtttttttcttgaggcagagtctctctctgtcgcccaggctggagtgcagtggcgccatctcagctcattgcaacctctgccacctccgggttcaagagattctcctgcctcagcctcccgagtagctgggattacaggtgtccaccaccacacccggctaattttttgtatttttagtagaggtggggtttcaccatgttggccaggctggtcttgagctcctgacctcaggtgatccacctgcctcggcctaccaaagagctgggataacaggtgtgacccaccatgcccggcccattttttttttcttattctgttaggagtgagagtgtaactagcagtataatagttcaattttcacaacgtggtaaaagtttccctataattcaatcagattttgctccagggttcagttctgttttaggaaatacttttattttcagtttaatgatgaaatattagagttgtaatattgcctttatgattatccacctttttaacctaaaagaatgaaagaaaaatatgtttgcaatataattttatggttgtatgttaacttaattcattatgttggcctccagtttgctgttgttagttatgacagcagtagtgtcattaccatttcaattcagattacattcctatatttgatcattgtaaactgactgcttacattgtattaaaaacagtggatattttaaagaagctgtacggcttatatctagtgctgtctcttaagactattaaattgatacaacatatttaaaagtaaatattacctaaatgaatttttgaaattacaaatacacgtgttaaaactgtcgttgtgttcaaccatttctgtacatacttagagttaactgttttgccaggctctgtatgcctactcataatatgataaaagcactcatctaatgctctgtaaatagaagtcagtgctttccatcagactgaactctcttgacaagatgtggatgaaattctttaagtaaaattgtttactttgtcatacatttacagatcaaatgttagctcccaaagcaatcatatggcaaagataggtatatcatagtttgcctattagctgctttgtattgctattattataaatagacttcacagttttagacttgcttaggtgaaattgcaattctttttactttcagtcttagataacaagtcttcaattatagtacaatcacacattgcttaggaatgcatcattaggcgattttgtcattatgcaaacatcatagagtgtacttacacaaacctagatagtatagcctttatgtacctaggccgtatggtatagtctgttgctcctaggccacaaacctgtacaactgttactgtactgaatactatagacagttgtaacacagtggtaaatatttatctaaatatatgcaaacagagaaaaggtacagtaaaagtatggtataaaagataatggtatacctgtgtaggccacttaccacgaatggagcttgcaggactagaagttgctctgggtgagtcagtgagtgagtggtgaattaatgtgaaggcctagaacactgtacaccactgtagactataaacacagtacgctgaagctacaccaaatttatcttaacagtttttcttcaataaaaaattataactttttaactttgtaaactttttaattttttaacttttaaaatacttagcttgaaacacaaatacattgtatagctatacaaaaatattttttctttgtatccttattctagaagcttttttctattttctattttaaatttttttttttacttgttagtcgtttttgttaaaaactaaaacacacacactttcacctaggcatagacaggattaggatcatcagtatcactcccttccacctcactgccttccacctccacatcttgtcccactggaaggtttttaggggcaataacacacatgtagctgtcacctatgataacagtgctttctgttgaatacctcctgaaggacttgcctgaggctgttttacatttaacttaaaaaaaaaaaaagtagaaggagtgcactctaaaataacaataaaaggcatagtatagtgaatacataaaccagcaatgtagtagtttattatcaagtgttgtacactgtaataattgtatgtgctatactttaaataacttgcaaaatagtactaagaccttatgatggttacagtgtcactaaggcaatagcatattttcaggtccattgtaatctaatgggactaccatcatatatgcagtctaccattgactgaaacgttacatggcacataactgtatttgcaagaatgatttgttttacattaatatcacataggatgtacctttttagagtggtatgtttatgtggattaagatgtacaagttgagcaaggggaccaagagccctgggttctgtcttggatgtgagcgtttatgttcttctcctcatgtctgttttctcattaaattcaaaggcttgaacgggccctatttagcccttctgttttctacgtgttctaaataactaaagcttttaaattctagccatttagtgtagaactctctttgcagtgatgaaatgctgtattggtttcttggctagcatattaaatatttttatctttgtcttgatacttcaatgtcgttttaaacatcaggatcgggcttcagtattctcataaccagagagttcactgaggatacaggactgtttgcccattttttgttatggctccagacttgtggtatttccatgtctttttttttttttttttttttgaccttttagcggctttaaagtatttctgttgttaggtgttgtattacttttctaagattacttaacaaagcaccacaaactgagtggctttaaacaacagcaatttattctctcacaattctagaagctagaagtccgaaatcaaagtgttgacaggggcatgatcttcaagagagaagactctttccttgcctcttcctggcttctggtggttaccagcaatcctgagtgttcctttcttgccttgtagtttcaacaatccagtatctgccttttgtcttcacatggctgtctaccatttgtctctgtgtctccaaatctctctccttataaacacagcagttattggattaggccccactctaatccagtatgaccccattttaacatgattacacttatttctagataaggtcacattcacgtacaccaagggttaggaattgaacatatctttttgggggacacaattcaacccacaagtgtcagtctctagctgagcctttcccttcctgtttttctcctttttagttgctatgggttaggggccaaatctccagtcatactagaattgcacatggactggatatttgggaatactgcgggtctattctatgagctttagtatgtaacatttaatatcagtgtaaagaagcccttttttaagttatttctttgaatttctaaatgtatgccctgaatataagtaacaagttaccatgtcttgtaaaatgatcatatcaacaaacatttaatgtgcacctactgtgctagttgaatgtctttatcctgataggagataacaggattccacatctttgacttaagaggacaaaccaaatatgtctaaatcatttggggttttgatggatatctttaaattgctgaacctaatcattggtttcatatgtcattgtttagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggtaagtgatttttcagcttattaatcatgttaacctatctgttgaaagcttattttctggtacatataaatcttatttttttaattatatgcagtgaacatcaaacaataaatgttatttattttgcatttaccctattagatacaaatacatctggtctgatacctgtcatcttcatattaactgtggaaggtacgaaatggtagctccacattatagatgaaaagctaaagcttagacaaataaagaaacttttagaccctggattcttcttgggagcctttgactctaataccttttgtttccctttcattgcacaattctgtcttttgcttactactatgtgtaagtataacagttcaaagtaatagtttcataagctgttggtcatgtagcctttggtctctttaacctctttgccaagttcccaggttcataaaatgaggaggttgaatggaatggttcccaagagaattccttttaatcttacagaaattattgttttcctaaatcctgtagttgaatatataatgctatttacatttcagtatagttttgatgtatctaaagaacacattgaattctccttcctgtgttccagtttgatactaacctgaaagtccattaagcattaccagttttaaaaggcttttgcccaatagtaaggaaaaataatatcttttaaaagaataattttttactatgtttgcaggcttacttccttttttctcacattatgaaactcttaaaatcaggagaatcttttaaacaacatcataatgtttaatttgaaaagtgcaagtcattcttttcctttttgaaactatgcagatgttacattgactgttttctgtgaagttatctttttttcactgcagaataaaggttgttttgattttattttgtattgtttatgagaacatgcatttgttgggttaatttcctacccctgcccccattttttccctaaagtagaaagtatttttcttgtgaactaaattactacacaagaacatgtctattgaaaaataagcaagtatcaaaatgttgtgggttgtttttttaaataaattttctcttgctcaggaaagacaagaaaatgtccagaagattatcttagaaggcacagagagaatggaagatcaggtatatgcaaattgcatactgtcaaatgtttttctcacagcatgtatctgtataaggttgatggctacatttgtcaaggccttggagacatacgaataagcctttaatggagcttttatggaggtgtacagaataaactggaggaagatttccatatcttaaacccaaagagttaaatcagtaaacaaaggaaaatagtaattgcatctacaaattaatatttgctccctttttttttctgtttgcccagaataaattttggataacttgttcatagtaaaaataaaaaaaattgtctctgatatgttctttaaggtactacttctcgaacctttccctagaagtagctgtaacagaaggagagcatatgtacccctgaggtatctgtctggggtgtaggcccaggtccacacaatatttcttctaagtcttatgttgtatcgttaagactcatgcaatttacattttattccataactattttagtattaaaatttgtcagtgatatttcttaccctctcctctaggaaaatgtgccatgtttatcccttggctttgaatgcccctcaggaacagacactaagagtttgagaagcatggttacaagggtgtggcttcccctgcggaaactaagtacagactatttcactgtaaagcagagaagttcttttgaaggagaatctccagtgaagaaagagttcttcacttttacttccatttcctcttgtgggtgaccctcaatgctccttgtaaaactccaatattttaaacatggctgttttgcctttctttgcttctttttagcatgaatgagacagatgatactttaaaaaagtaattaaaaaaaaaaacttgtgaaaatacatggccataatacagaacccaatacaatgatctcctttaccaaattgttatgtttgtacttttgtagatagctttccaattcagagacagttattctgtgtaaaggtctgacttaacaagaaaagatttccctttacccaaagaatcccagtccttatttgctggtcaataagcagggtccccaggaatggggtaactttcagcaccctctaacccactagttattagtagactaattaagtaaacttatcgcaagttgaggaaacttagaaccaactaaaattctgcttttactgggattttgttttttcaaaccagaaacctttacttaagttgactactattaatgaattttggtctctcttttaagtgctcttcttaaaaatgttatcttactgctgagaagttcaagtttgggaagtacaaggaggaatagaaacttaagagattttcttttagagcctcttctgtatttagccctgtaggatttttttttttttttttttttttggtgttgttgagcttcagtgaggctattcattcacttatactgataatgtctgagatactgtgaatgaaatactatgtatgcttaaacctaagaggaaatattttcccaaaattattcttcccgaaaaggaggagttgccttttgattgagttcttgcaaatctcacaacgactttattttgaacaatactgtttggggatgatgcattagtttgaaacaacttcagttgtagctgtcatctgataaaattgcttcacagggaaggaaatttaacacggatctagtcattattcttgttagattgaatgtgtgaattgtaattgtaaacaggcatgataattattactttaaaaactaaaaacagtgaatagttagttgtggaggttactaaaggatggtttttttttaaataaaactttcagcattatgcaaatgggcatatggcttaggataaaacttccagaagtagcatcacatttaaattctcaagcaacttaataatatggggctctgaaaaactggttaaggttactccaaaaatggccctgggtctgacaaagattctaacttaaagatgcttatgaagactttgagtaaaatcatttcataaaataagtgaggaaaaacaactagtattaaattcatcttaaataatgtatgatttaaaaaatatgtttagctaaaaatgcatagtcatttgacaatttcatttatatctcaaaaaatttacttaaccaagttggtcacaaaactgatgagactggtggtggtagtgaataaatgagggaccatccatatttgagacactttacatttgtgatgtgttatactgaattttcagtttgattctatagactacaaatttcaaaattacaatttcaagatgtaataagtagtaatatcttgaaatagctctaaagggaatttttctgttttattgattcttaaaatatatgtgctgattttgatttgcatttgggtagattatacttttatgagtatggaggttaggtattgattcaagttttccttacctatttggtaaggatttcaaagtctttttgtgcttggttttcctcatttttaaatatgaaatatattgatgacctttaacaaattttttttatctcaaattttaaaggagatcttttctaaaagaggcatgatgacttaatcattgcatgtaacagtaaacgataaaccaatgattccatactctctaaagaataaaagtgagctttagggccgggcatggtcagaaatttgacaccaacctggccaacatggcgaaaccccgtctctactaaaaatacaaaaatcagccgggcatggtggcggcacctatagtcccagctacttgggaggatgagacaggagagtcacttgaacctgggaggagaggttgcagtgagctgagatcacgccattgcactccagcctgagcaatgaaagcaaaactccatctcaaaaaaaaaaaaagaaaagaaagaataaaagtgagctttggattgcatataaatcctttagacatgtagtagacttgtttgatactgtgtttgaacaaattacgaagtattttcatcaaagaatgttattgtttgatgttatttttattttttattgcccagcttctctcatattacgtgattttcttcacttcatgtcactttattgtgcagggtcagagtattattccaatgcttactggagaagtgattcctgtaatggaactgctttcatctatgaaatcacacagtgttcctgaagaaatagatgtaagtttaaatgagagcaattatacactttatgagttttttggggttatagtattattatgtatattattaatattctaattttaatagtaaggactttgtcatacatactattcacatacagtattagccactttagcaaataagcacacacaaaatcctggattttatggcaaaacagaggcatttttgatcagtgatgacaaaattaaattcattttgtttatttcattacttttataattcctaaaagtgggaggatcccagctcttataggagcaattaatatttaatgtagtgtcttttgaaacaaaactgtgtgccaaagtagtaaccattaatggaagtttacttgtagtcacaaatttagtttccttaatcatttgttgaggacgttttgaatcacacactatgagtgttaagagatacctttaggaaactattcttgttgttttctgattttgtcatttaggttagtctcctgattctgacagctcagaagaggaagttgttcttgtaaaaattgtttaacctgcttgaccagctttcacatttgttcttctgaagtttatggtagtgcacagagattgttttttggggagtcttgattctcggaaatgaaggcagtgtgttatattgaatccagacttccgaaaacttgtatattaaaagtgttatttcaacactatgttacagccagactaatttttttattttttgatgcattttagatagctgatacagtactcaatgatgatgatattggtgacagctgtcatgaaggctttcttctcaagtaagaatttttcttttcataaaagctggatgaagcagataccatcttatgctcacctatgacaagatttggaagaaagaaaataacagactgtctacttagattgttctagggacattacgtatttgaactgttgcttaaatttgtgttatttttcactcattatatttctatatatatttggtgttattccatttgctatttaaagaaaccgagtttccatcccagacaagaaatcatggccccttgcttgattctggtttcttgttttacttctcattaaagctaacagaatcctttcatattaagttgtactgtagatgaacttaagttatttaggcgtagaacaaaattattcatatttatactgatctttttccatccagcagtggagtttagtacttaagagtttgtgcccttaaaccagactccctggattaatgctgtgtacccgtgggcaaggtgcctgaattctctatacacctatttcctcatctgtaaaatggcaataatagtaatagtacctaatgtgtagggttgttataagcattgagtaagataaataatataaagcacttagaacagtgcctggaacataaaaacacttaataatagctcatagctaacatttcctatttacatttcttctagaaatagccagtatttgttgagtgcctacatgttagttcctttactagttgctttacatgtattatcttatattctgttttaaagtttcttcacagttacagattttcatgaaattttacttttaataaaagagaagtaaaagtataaagtattcacttttatgttcacagtcttttcctttaggctcatgatggagtatcagaggcatgagtgtgtttaacctaagagccttaatggcttgaatcagaagcactttagtcctgtatctgttcagtgtcagcctttcatacatcattttaaatcccatttgactttaagtaagtcacttaatctctctacatgtcaatttcttcagctataaaatgatggtatttcaataaataaatacattaattaaatgatattatactgactaattgggctgttttaaggctcaataagaaaatttctgtgaaaggtctctagaaaatgtaggttcctatacaaataaaagataacattgtgcttatagcttcggtgtttatcatataaagctattctgagttatttgaagagctcacctacttttttttgtttttagtttgttaaattgttttataggcaatgtttttaatctgttttctttaacttacagtgccatcagctcacacttgcaaacctgtggctgttccgttgtagtaggtagcagtgcagagaaagtaaataaggtagtttattttataatctagcaaatgatttgactctttaagactgatgatatatcatggattgtcatttaaatggtaggttgcaattaaaatgatctagtagtataaggaggcaatgtaatctcatcaaattgctaagacaccttgtggcaacagtgagtttgaaataaactgagtaagaatcatttatcagtttattttgatagctcggaaataccagtgtcagtagtgtataaatggttttgagaatatattaaaatcagatatataaaaaaaattactcttctatttcccaatgttatctttaacaaatctgaagatagtcatgtacttttggtagtagttccaaagaaatgttatttgtttattcatcttgatttcattgtcttcgctttccttctaaatctgtcccttctagggagctattgggattaagtggtcattgattattatactttattcagtaatgtttctgaccctttccttcagtgctacttgagttaattaaggattaatgaacagttacatttccaagcattagctaataaactaaaggattttgcacttttcttcactgaccattagttagaaagagttcagagataagtatgtgtatctttcaatttcagcaaacctaattttttaaaaaaagttttacataggaaatatgttggaaatgatactttacaaagatattcataatttttttttgtaatcagctactttgtatatttacatgagccttaatttatatttctcatataaccatttatgagagcttagtatacctgtgtcattatattgcatctacgaactagtgaccttattccttctgttacctcaaacaggtggctttccatctgtgatctccaaagccttaggttgcacagagtgactgccgagctgctttatgaagggagaaaggctccatagttggagtgtttttttttttttttttaaacatttttcccatcctccatcctcttgagggagaatagcttaccttttatcttgttttaatttgagaaagaagttgccaccactctaggttgaaaaccactcctttaacataataactgtggatatggtttgaatttcaagatagttacatgcctttttatttttcctaatagagctgtaggtcaaatattattagaatcagatttctaaatcccacccaatgacctgcttattttaaatcaaattcaataattaattctcttctttttggaggatctggacattctttgatatttcttacaacgaatttcatgtgtagacccactaaacagaagctataaaagttgcatggtcaaataagtctgagaaagtctgcagatgatataattcacctgaagagtcacagtatgtagccaaatgttaaaggttttgagatgccatacagtaaatttaccaagcattttctaaatttatttgaccacagaatccctattttaagcaacaactgttacatcccatggattccaggtgactaaagaatacttatttcttaggatatgttttattgataataacaattaaaatttcagatatctttcataagcaaatcagtggtctttttacttcatgttttaatgctaaaatattttcttttatagatagtcagaacattatgcctttttctgactccagcagagagaaaatgctccaggttatgtgaagcagaatcatcatttaaatatgagtcagggctctttgtacaaggcctgctaaaggtatagtttctagttatcacaagtgaaaccacttttctaaaatcatttttgagactctttatagacaaatcttaaatattagcatttaatgtatctcatattgacatgcccagagactgacttcctttacacagttctgcacatagactatatgtcttatggatttatagttagtatcatcagtgaaacaccatagaataccctttgtgttccaggtgggtccctgttcctacatgtctagcctcaggacttttttttttttaacacatgcttaaatcaggttgcacatcaaaaataagatcatttctttttaactaaatagatttgaattttattgaaaaaaaattttaaacatctttaagaagcttataggatttaagcaattcctatgtatgtgtactaaaatatatatatttctatatataatatatattagaaaaaaattgtatttttcttttatttgagtctactgtcaaggagcaaaacagagaaatgtaaattagcaattatttataatacttaaagggaagaaagttgttcaccttgttgaatctattattgttatttcaattatagtcccaagacgtgaagaaatagctttcctaatggttatgtgattgtctcatagtgactactttcttgaggatgtagccacggcaaaatgaaataaaaaaatttaaaaattgttgcaaatacaagttatattaggcttttgtgcattttcaataatgtgctgctatgaactcagaatgatagtatttaaatatagaaactagttaaaggaaacgtagtttctatttgagttatacatatctgtaaattagaacttctcctgttaaaggcataataaagtgcttaatacttttgtttcctcagcaccctctcatttaattatataattttagttctgaaagggacctataccagatgcctagaggaaatttcaaaactatgatctaatgaaaaaatatttaatagttctccatgcaaatacaaatcatatagttttccagaaaatacctttgacattatacaaagatgattatcacagcattataatagtaaaaaaatggaaatagcctctttcttctgttctgttcatagcacagtgcctcatacgcagtaggttattattacatggtaactggctaccccaactgattaggaaagaagtaaatttgttttataaaaatacatactcattgaggtgcatagaataattaagaaattaaaagacacttgtaattttgaatccagtgaatacccactgttaatatttggtatatctctttctagtctttttttcccttttgcatgtattttctttaagactcccacccccactggatcatctctgcatgttctaatctgcttttttcacagcagattctaagcctctttgaatatcaacacaaacttcaacaacttcatctatagatgccaaataataaattcatttttatttacttaaccacttcctttggatgcttaggtcattctgatgttttgctattgaaaccaatgctatactgaacacttctgtcactaaaactttgcacacactcatgaatagcttcttaggataaatttttagagatggatttgctaaatcagagaccattttttaaaattaaaaaacaattattcatatcgtttggcatgtaagacagtaaattttccttttattttgacaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgtacgtaatgctctgcctgctggtactgtagtcaagcaatatgaaattgtgtcttttacgaataaaaacaaaacagaagttgcatttaaaaagaaagaaatattaccagcagaattatgcttgaagaaacatttaatcaagcatttttttcttaaatgttcttctttttccatacaattgtgtttaccctaaaataggtaagattaacccttaaagtaaatatttaactatttgtttaataaatatatattgagctcctaggcactgttctaggtaccgggcttaatagtggccaaccagacagccccagccccagcccctacattgtgtatagtctattatgtaacagttattgaatggacttattaacaaaaccaaagaagtaattctaagtcttttttttcttgacatatgaatataaaatacagcaaaactgttaaaatatattaatggaacatttttttactttgcattttatattgttattcacttcttatttttttttaaaaaaaaaagcctgaacagtaaattcaaaaggaaaagtaatgataattaattgttgagcatggacccaacttgaaaaaaaaaatgatgatgataaatctataatcctaaaaccctaagtaaacacttaaaagatgttctgaaatcaggaaaagaattatagtatacttttgtgtttctcttttatcagttgaaaaaaggcacagtagctcatgcctgtaagaacagagctttgggagtgcaaggcaggcggatcacttgaggccaggagttccagaccagcctgggcaacatagtgaaaccccatctctacaaaaaataaaaaagaattattggaatgtgtttctgtgtgcctgtaatcctagctattccgaaagctgaggcaggaggatcttttgagcccaggagtttgaggttacagggagttatgatgtgccagtgtactccagcctggggaacaccgagactctgtcttatttaaaaaaaaaaaaaaaaaaatgcttgcaataatgcctggcacatagaaggtaacagtaagtgttaactgtaataacccaggtctaagtgtgtaaggcaatagaaaaattggggcaaataagcctgacctatgtatctacagaatcagtttgagcttaggtaacagacctgtggagcaccagtaattacacagtaagtgttaaccaaaagcatagaataggaatatcttgttcaagggacccccagccttatacatctcaaggtgcagaaagatgacttaatataggacccattttttcctagttctccagagtttttattggttcttgagaaagtagtaggggaatgttttagaaaatgaattggtccaactgaaattacatgtcagtaagtttttatatattggtaaattttagtagacatgtagaagttttctaattaatctgtgccttgaaacattttcttttttcctaaagtgcttagtattttttccgttttttgattggttacttgggagcttttttgaggaaatttagtgaactgcagaatgggtttgcaaccatttggtatttttgttttgttttttagaggatgtatgtgtattttaacatttcttaatcatttttagccagctatgtttgttttgctgatttgacaaactacagttagacagctattctcattttgctgatcatgacaaaataatatcctgaatttttaaattttgcatccagctctaaattttctaaacataaaattgtccaaaaaatagtattttcagccactagattgtgtgttaagtctattgtcacagagtcattttacttttaagtatatgtttttacatgttaattatgtttgttatttttaattttaactttttaaaataattccagtcactgccaatacatgaaaaattggtcactggaattttttttttgacttttattttaggttcatgtgtacatgtgcaggtgtgttatacaggtaaattgcgtgtcatgagggtttggtgtacaggtgatttcattacccaggtaataagcatagtacccaataggtagttttttgatcctcacccttctcccaccctcaagtaggccctggtgttgctgtttccttctttgtgtccatgtatactcagtgtttagctcccacttagaagtgagaacatgcggtagttggttttctgttcctggattagttcacttaggataatgacctctagctccatctggtttttatggctgcatagtattccatggtgtatatgtatcacattttctttatccagtctaccattgataggcatttaggttgattccctgtctttgttatcatgaatagtgctgtgatgaacatacacatgcatgtgtctttatggtagaaaaatttgtattcctttaggtacatatagaataatggggttgctagggtgaatggtagttctattttcagttatttgagaaatcttcaaactgcttttcataatagctaaactaatttacagtcccgccagcagtgtataagtgttcccttttctccacaaccttgccaacatctgtgattttttgactttttaataatagccattcctagagaattgatttgcaattctctattagtgatattaagcattttttcatatgctttttagctgtctgtatatattcttctgaaaaattttcatgtcctttgcccagtttgtagtggggtgggttgttttttgcttgttaattagttttaagttccttccagattctgcatatccctttgttggatacatggtttgcagatatttttctcccattgtgtaggttgtcttttactctgttgatagtttcttttgccatgcaggagctcgttaggtcccatttgtgtttgtttttgttgcagttgcttttggcgtcttcatcataaaatctgtgccagggcctatgtccagaatggtatttcctaggttgtcttccagggtttttacaattttagattttacgtttatgtctttaatccatcttgagttgatttttgtatatggcacaaggaaggggtccagtttcactccaattcctatggctagcaattatcccagcaccatttattgaatacggagtcctttccccattgcttgttttttgtcaactttgttgaagatcagatggttgtaagtgtgtggctttatttcttggctctctattctccattggtctatgtgtctgtttttataacagtaccctgctgttcaggttcctatagccttttagtataaaatcggctaatgtgatgcctccagctttgttctttttgcttaggattgctttggctatttgggctcctttttgggtccatattaattttaaaacagttttttctggttttgtgaaggatatcattggtagtttataggaatagcattgaatctgtagattgctttgggcagtatggccattttaacaatattaattcttcctatctatgaatatggaatgtttttccatgtgtttgtgtcatctctttatacctgatgtataaagaaaagctggtattattcctactcaatctgttccaaaaaattgaggaggaggaactcttccctaatgaggccagcatcattctgataccaaaacctggcagagacacaacagaaaaaagaaaacttcaggccaatatccttgatgaatatagatgcaaaaatcctcaacaaaatactagcaaaccaaatccagcagcacatcaaaaagctgatctactttgatcaagtaggctttatccctgggatgcaaggttggttcaacatacacaaatcaataagtgtgattcatcacataaacagagctaaaaacaaaaaccacaagattatctcaataggtagagaaaaggttgtcaataaaatttaacatcctccatgttaaaaaccttcagtaggtcaggtgtagtgactcacacctgtaatcccagcactttgggaggccaaggcgggcatatctcttaagcccaggagttcaagacgagcctaggcagcatggtgaaaccccatctctacaaaaaaaaaaaaaaaaaaaaattagcttggtatggtgacatgcacctatagtcccagctattcaggaggttgaggtgggaggattgtttgagcccgggaggcagaggttggcagcgagctgagatcatgccaccgcactccagcctgggcaacggagtgagaccctgtctcaaaaaagaaaaatcacaaacaatcctaaacaaactaggcattgaaggaacatgcctcaaaaaaataagaaccatctatgacagacccatagccaatatcttaccaaatgggcaaaagctggaagtattctccttgagaaccgtaacaagacaaggatgtccactctcaccactccttttcagcatagttctggaagtcctagccagagcaatcaggaaagagaaagaaagaaagacattcagataggaagagaagaagtcaaactatttctgtttgcaggcagtataattctgtacctagaaaatctcatagtctctgcccagaaactcctaaatctgttaaaaatttcagcaaagttttggcattctctatactccaacaccttccaaagtgagagcaaaatcaagaacacagtcccattcacaatagccgcaaaacgaataaaatacctaggaatccagctaaccagggaggtgaaagatctctatgagaattacaaaacactgctgaaagaaatcagagatgacacaaacaaatggaaatgttcttttttaacaccttgctttatctaattcacttatgatgaagatactcattcagtggaacaggtataataagtccactcgattaaatataagccttattctctttccagagcccaagaaggggcactatcagtgcccagtcaataatgacgaaatgctaatatttttcccctttacggtttctttcttctgtagtgtggtacactcgtttcttaagataaggaaacttgaactaccttcctgtttgcttctacacatacccattctctttttttgccactctggtcaggtataggatgatccctaccactttcagttaaaaactcctcctcttactaaatgttctcttaccctctggcctgagtagaacctagggaaaatggaagagaaaaagatgaaagggaggtggggcctgggaagggaataagtagtcctgtttgtttgtgtgtttgctttagcacctgctatatcctaggtgctgtgttaggcacacattattttaagtggccattatattactactactcactctggtcgttgccaaggtaggtagtactttcttggatagttggttcatgttacttacagatggtgggcttgttgaggcaaacccagtggataatcatcggagtgtgttctctaatctcactcaaatttttcttcacattttttggtttgttttggtttttgatggtagtggcttatttttgttgctggtttgttttttgtttttttttgagatggcaagaattggtagttttatttattaattgcctaagggtctctactttttttaaaagatgagagtagtaaaatagattgatagatacatacatacccttactggggactgcttatattctttagagaaaaaattacatattagcctgacaaacaccagtaaaatgtaaatatatccttgagtaaataaatgaatgtatattttgtgtctccaaatatatatatctatattcttacaaatgtgtttatatgtaatatcaatttataagaacttaaaatgttggctcaagtgagggattgtggaaggtagcattatatggccatttcaacatttgaacttttttcttttcttcattttcttcttttcttcaggaatatttttcaagatgtcttacacagagacactctagtgaaagccttcctggatcaggtaaatgttgaacttgagattgtcagagtgaatgatatgacatgttttcttttttaatatatcctacaatgcctgttctatatatttatattcccctggatcatgccccagagttctgctcagcaattgcagttaagttagttacactacagttctcagaagagtctgtgagggcatgtcaagtgcatcattacattggttgcctcttgtcctagatttatgcttcgggaattcagacctttgtttacaatataataaatattattgctatcttttaaagatataataataagatataaagttgaccacaactactgttttttgaaacatagaattcctggtttacatgtatcaaagtgaaatctgacttagcttttacagatataatatatacatatatatatcctgcaatgcttgtactatatatgtagtacaagtatatatatatgtttgtgtgtgtatatatatatagtacgagcatatatacatattaccagcattgtaggatatatatatgtttatatattaaaaaaaagttataaacttaaaaccctattatgttatgtagagtatatgttatatatgatatgtaaaatatataacatatactctatgatagagtgtaatatattttttatatatattttaacatttataaaatgatagaattaagaattgagtcctaatctgttttattaggtgctttttgtagtgtctggtctttctaaagtgtctaaatgatttttccttttgacttattaatggggaagagcctgtatattaacaattaagagtgcagcattccatacgtcaaacaacaaacattttaattcaagcattaacctataacaagtaagttttttttttttttttgagaaagggaggttgtttatttgcctgaaatgactcaaaaatatttttgaaacatagtgtacttatttaaataacatctttattgtttcattcttttaaaaaatatctacttaattacacagttgaaggaaatcgtagattatatggaacttatttcttaatatattacagtttgttataataacattctggggatcaggccaggaaactgtgtcatagataaagctttgaaataatgagatccttatgtttactagaaattttggattgagatctatgaggtctgtgacatattgcgaagttcaaggaaaattcgtaggcctggaatttcatgcttctcaagctgacataaaatccctcccactctccacctcatcatatgcacacattctactcctacccacccactccaccccctgcaaaagtacaggtatatgaatgtctcaaaaccataggctcatcttctaggagcttcaatgttatttgaagatttgggcagaaaaaattaagtaatacgaaataacttatgtatgagttttaaaagtgaagtaaacatggatgtattctgaagtagaatgcaaaatttgaatgcatttttaaagataaattagaaaacttctaaaaactgtcagattgtctgggcctggtggcttatgcctgtaatcccagcactttgggagtccgaggtgggtggatcacaaggtcaggagatcgagaccatcctgccaacatggtgaaaccccgtctctactaagtatacaaaaattagctgggcgtggcagcgtgtgcctgtaatcccagctacctgggaggctgaggcaggagaatcgcttgaacccaggaggtgtaggttgcagtgagtcaagatcgcgccactgcactttagcctggtgacagagctagactccgtctcaaaaaaaaaaaaaaatatcagattgttcctacacctagtgcttctataccacactcctgttagggggcatcagtggaaatggttaaggagatgtttagtgtgtattgtctgccaagcactgtcaacactgtcatagaaacttctgtacgagtagaatgtgagcaaattatgtgttgaaatggttcctctccctgcaggtctttcagctgaaacctggcttatctctcagaagtactttccttgcacagtttctacttgtccttcacagaaaagccttgacactaataaaatatatagaagacgatacgtgagtaaaactcctacacggaagaaaaacctttgtacattgtttttttgttttgtttcctttgtacattttctatatcataatttttgcgcttcttttttttttttttttttttttttttccattatttttaggcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaattttg

SEQ ID NO: 57 - DNA, 5502 bp, Artificial Sequence
  S92RNL (Sense GR-Nanoluciferase reporter plasmid)
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactatagggatatctgcttatcgataccgtcgacctcgaatcactagtcagctggaattcctcacagtactcgctgagggtgaacaagaaaagacctgataaagattaaccagaagaaaacaaggagggaaacaaccgcagcctgtagcaagctctggaactcaggagtcgcgcgctagctcttcaggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggcctgcggccgctggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtactggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataggtaccgagctcttacgcgtgctagcccgggctcgag

SEQ ID NO: 58 - DNA, 6099 bp, Artificial Sequence
  AS55RNL Nanoluciferase reporter plasmid
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactatagggatatcgaattcaatagtcacttcctttaagcaagtctgtgtcatctcggagctgtgaagcaaccaggtcatgtcccacagaatggggagcacaccgacttgcattgctgccctcatatgcaagtcatcaccactctctagaagcttgggctgaaattgtgcaggcgtctccacacccccatctcatcccgcatgatctcctcgccggcagggaccgtctcgggttcctagcgaaccccgacttggtccgcagaagccgcgcgccgcccaccctccggccttcccccaggcgaggcctctcagtacccgaggctcccttttctcgagcccgcagcggcagcgctcccagcgggtccccgggaaggagacagctcgggtactgagggcgggaaagcaaggaagaggccagatccccatcccttgtccctgcgccgccgccgccgccgccgccgccgggaagccgaattccggggcccggatgcaggcaattccaccagtcgctagaggcgaaagcccgacacccagcttcggtcagagaaatgagagggaaagtaaaaatgcgtcgagctctgaggagagcccccgcttctacccgcgcctcttcccggcagccgaaccccaaacagccacccgccaggatgccgcctcctcactcacccactcgccaccgcctgcgcctccgccgccgcgggcgcaggcaccgcaaccgcagccccgccccgggcccgcccccgggcccgccccgaccacgagcggccgcaggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccccggccactagtcagctggaattggccgctggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtactggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataggtaccgagctcttacgcgtgctagcccgggctcgag

SEQ ID NO: 59 - DNA, 4944 bp, Artificial Sequence
  S0RNL Nanoluciferase reporter plasmid
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactatagggatatctgcttatcgataccgtcgacctcgaatcactagtcagctggaattcctcacagtactcgctgagggtgaacaagaaaagacctgataaagattaaccagaagaaaacaaggagggaaacaaccgcagcctgtagcaagctctggaactcaggagtcgcgcgctagggggctctggccgctggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtactggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataggtaccgagctcttacgcgtgctagcccgggctcgag

SEQ ID NO: 60 - DNA, 120 bp, Artificial Sequence
  Base pairs 201-320 of the C9orf72 gene (SEQ ID NO 56)
ctctcacagtactcgctgagggtgaacaagaaaagacctgataaagattaaccagaagaaaacaaggagggaaacaaccgcagcctgtagcaagctctggaactcaggagtcgcgcgcta

SEQ ID NO: 61 - DNA, 157 bp, Artificial Sequence
  Base pairs 418-574 of the C9orf72 gene (SEQ ID NO 56)
cgcaggcggtggcgagtgggtgagtgaggaggcggcatcctggcgggtggctgtttggggttcggctgccgggaagaggcgcgggtagaagcgggggctctcctcagagctcgacgcatttttactttccctctcatttctctgaccgaagctgggt

SEQ ID NO: 62 - DNA, 6 bp, Artificial Sequence
  Hexanucleotide repeat (sense)
ggggcc

SEQ ID NO: 63 - DNA, 6 bp, Artificial Sequence
  Hexanucleotide repeat (antisense)
ccccgg

SEQ ID NO: 64 - protein, 959 aa, Ruminococcus flavefaciens
IEKKKSFAKGMGVKSTLVSGSKVYMTTFAEGSDARLEKIVEGDSIRSVNEGEAFSAEMADKNAGYKIGNAKFSHPKGYAWANNPLYTGPVQQDMLGLKETLEKRYFGESADGNDNICIQVIHNILDIEKILAEYITNMYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSMNVNYIAETLGINPAEFAEQYFRFSIMKEQKNLGFNITKLREVMLDRKDMSEIRKNHKVFDSIRTKVYTMMDFVIYRYYIEEDAKVAMNKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEMKFDKEKKSGNS

SEQ ID NO: 65 - protein, 1000 aa, Artificial Sequence
  Example Cas13Rx polypeptide sequence
MSPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYA

SEQ ID NO: 66 - DNA, 6 bp, Artificial Sequence
  Alternative hexanucleotide repeat (antisense)
ggcccc

SEQ ID NO: 67 - DNA, 20 bp, Artificial Sequence
  Repeat containing intron 1a Fwd primer
ccccactacttgctctcaca

SEQ ID NO: 68 - DNA, 20 bp, Artificial Sequence
  Repeat containing intron 1a Rev primer
cggttgtttccctccttgtt

SEQ ID NO: 69 - DNA, 18 bp, Artificial Sequence
  Exon 1b containing Fwd primer
gcggtggcgagtggatat

SEQ ID NO: 70 - DNA, 20 bp, Artificial Sequence
  Exon 1b containing Rev primer
tgggcaaagagtcgacatca

SEQ ID NO: 71 - DNA, 19 bp, Artificial Sequence
  Antisense repeat transcript Fwd primer
agtcgctagaggcgaaagc

SEQ ID NO: 72 - DNA, 19 bp, Artificial Sequence
  Antisense repeat containing Rev primer
cgagtgggtgagtgaggag

SEQ ID NO: 73 - DNA, 22 bp, Artificial Sequence
  GAPDH Fwd primer
gtctcctctgacttcaacagcg

SEQ ID NO: 74 - DNA, 22 bp, Artificial Sequence
  GAPDH Rev primer
accaccctgttgctgtagccaa

SEQ ID NO: 75 - DNA, 23 bp, Artificial Sequence
  149R AAV repeats Fwd primer
agtactcgctgagggtgaacaag

SEQ ID NO: 76 - DNA, 22 bp, Artificial Sequence
  149R AAV repeats Rev primer
agcttgctacaggctgcggttg

SEQ ID NO: 77 - DNA, 30 bp, Artificial Sequence
  Non-targeting guide sequence
gtaatgcctggcttgtcgacgcatagtctg